(19) United States
(12) Patent Application Publication
(10) Pub. No.: US 2025/0127940 A1
(43) Pub. Date: Apr. 24, 2025

(54) RADIOPHARMACEUTICAL AND METHODS

(71) Applicant: POINT BIOPHARMA, INC., Indianapolis, IN (US)

(72) Inventors: Joe MCCANN, Toronto (CA); Mark DORNAN, Toronto (CA)

(21) Appl. No.: 18/934,324

(22) Filed: Nov. 1, 2024

Related U.S. Application Data
(63) Continuation of application No. PCT/US2023/020754, filed on May 2, 2023.
(60) Provisional application No. 63/337,480, filed on May 2, 2022.

(51) Int. Cl. A61K 51/04

(52) U.S. Cl. CPC A61K 51/0497; A61K 51/0406; A61K 2121/00

(57) ABSTRACT

Radiopharmaceutical compositions with extended shelf life are provided, including radiopharmaceutical agents that include one or aryl-halide moieties and formulated with a gentisate compound.

### CROSS-REFERENCES TO RELATED APPLICATIONS

This application is a continuation of International Application No. PCT/US2023/020754, which designated the United States and was filed on May 2, 2023, which claims the benefit of priority of U.S. Provisional Application No. 63/337,480 filed on May 2, 2022, which is incorporated herein by reference in its entirety and for all purposes.

### TECHNICAL FIELD

Radiopharmaceutical compositions with extended shelf life are provided, including radiopharmaceutical agents that include one or more aryl-halide moieties and formulated with a gentisate compound.

### BACKGROUND

Radiopharmaceuticals have been used for a variety of therapeutic and diagnostic indications. Among others, radiolabeled molecules have been useful to treat various malignant tumors.

Use of these pharmaceutical agents presents certain challenges, including with respect to the formation of the active pharmaceutical ingredient by combination of a radionuclide with a targeting agent whereby the resulting active pharmaceutical ingredient has low purity. Furthermore, the subsequent formulation of the active pharmaceutical ingredient to form a pharmaceutical agent has poor stability and decreased shelf-life. In particular, therapeutic compositions comprising a radionuclide may undergo radiolysis during any one or more of formation of the active pharmaceutical ingredient, preparation of the pharmaceutical agent and storage of a pharmaceutical composition. During radiolysis, radionuclide emissions may react with other groups of the active pharmaceutical ingredient thereby resulting in decomposition of the active pharmaceutical ingredient and a reduction in purity which limits the shelf-life and clinical usefulness of the pharmaceutical composition.

It thus would be desirable to have new pharmaceutical agents. It would be particularly desirable to have such agents that exhibit improved purities, stability and shelf-lives.

### SUMMARY

In one aspect, we have now found new radiopharmaceutical formulations that can exhibit extended shelf life. Preferred formulations include those that maintain greater than 95% radiochemical purity at 20° C., 25° C. or even 30° C. for a period of at least three days after preparation of the formulation, and even more preferably four, five, six or even seven days after formulation. In certain embodiments, the formulation maintains at least 96%, 97% or even 98% radiochemical purity.

In one aspect, aqueous pharmaceutical formulations which are provided comprise: 1) a radiopharmaceutical compound that comprises one or more aryl-halide moieties; and 2) one or more gentisate compounds.

In one aspect, preferred radiopharmaceutical compounds include those that comprise a structure of the following Formula (X):

Ch-L-TM (X)
wherein
   Ch represents a chelating moiety,
   TM represents a target moiety, and
   L comprises a linker including one or more aryl-halide moieties (i.e., an aryl group substituted with at least one halogen (e.g., —Cl, —I, —Br, —F, —At, or isotope thereof));
wherein the compound has a radioisotope (e.g., metal such as, to illustrate, ¹⁷⁷Lu, ²²⁵Ac, ²¹²Pb, ¹⁶¹Tb or ⁶⁷Cu, including metal ions) chelated therewith through the chelating moiety.

It has been found that in at least certain systems the presence of one or more gentisate compounds can suppress or otherwise reduce certain impurities that may occur in the absence of the one or more gentisate compounds. In at least certain systems, the presence of one or more gentisate compounds may suppress or otherwise reduce the formation of a de-halogenation derivative (e.g. a de-iodination, de-bromination or de-fluorinated derivative) of the original halogenated version of the radiopharmaceutical compound.

In one aspect, the radiopharmaceutical compound may comprise a halogenated phenyl moiety having one or more iodo, fluoro and/or bromo ring substituents. In an aspect, the phenyl moiety may comprise 1 or 2 halo substituents, such as 1 or 2 iodo, fluoro or bromo substituents, including 4-halophenyl substituent. In certain aspects, preferred radiopharmaceutical compounds may comprise a para-iodo-phenyl moiety including 4-(p-iodophenyl) butyryl.

In certain aspects, the radiopharmaceutical compound does not comprise a prostate-specific membrane antigen (PSMA)-ligand. In other aspects, the radiopharmaceutical compound does comprise a PSMA-ligand.

In certain aspects, the radiopharmaceutical compound comprises a moiety that can exhibit binding affinity for human serum albumin (HSA).

In certain aspects, the radiopharmaceutical compound comprises of ¹⁷⁷Lu, ²²⁵Ac, ²¹¹At, ¹⁶¹Tb, ⁶⁷Ga, ²⁰³Pb, ²¹²Pb, ²²³Ra and/or ¹³¹I. In certain aspects, the radiopharmaceutical compound comprises ¹⁷⁷Lu but not ²²⁵Ac, ²¹¹At, ⁶⁷Cu, ¹⁶¹Tb, ²¹²Pb or ¹³¹I. In certain aspects, the radiopharmaceutical compound comprises ²²⁵Ac but not ¹⁷⁷Lu, ⁶⁷Cu, ¹⁶¹Tb, ²¹²Pb or ¹³¹I. In certain aspects, the radiopharmaceutical compound comprises ¹³¹I, but not ²²⁵Ac, ²¹¹At, ⁶⁷Cu, ¹⁶¹Tb, ²¹²Pb or ¹⁷⁷Lu. In certain aspects, the radiopharmaceutical compound comprises ²¹¹At, but not ¹³¹I, ²²⁵Ac, ⁶⁷Cu, ¹⁶¹Tb, ²¹²Pb or ¹⁷⁷Lu. In certain aspects, the radiopharmaceutical compound comprises ⁶⁷Cu, but not ¹³¹I, ²²⁵Ac, ²¹¹At, ¹⁶¹Tb, ²¹²Pb or ¹⁷⁷Lu. In certain aspects, the radiopharmaceutical compound comprises ¹⁶¹Tb, but not ¹³¹I, ²²⁵Ac, ²¹¹At, ⁶⁷Cu, ²¹²Pb or ¹⁷⁷Lu. In certain aspects, the radiopharmaceutical compound comprises ²¹²Pb, but not ¹³¹I, ²²⁵Ac, ²¹¹At, ⁶⁷Cu, ¹⁶¹Tb or ¹⁷⁷Lu.

In certain aspects, the radiopharmaceutical compound is ²²⁵Ac-PSMA I & T, ⁶⁷Cu-PSMA I & T, ¹⁶¹Tb-PSMA I & T, or ²¹²Pb-PSMA I & T.

In certain aspects, the pharmaceutical compositions further comprise one or more ascorbate compounds.

In certain aspects, the one or more gentisate compounds such as gentisic acid are present in a pharmaceutical composition in an amount of at least 15 mg/mL, such as 16, 18, 20, 25, 30, 40, 50, 60 or 70 mg/mL or more. In certain systems, lower amounts of one or more gentisate compounds may be present in a pharmaceutical composition, such as up to or less than 14, 13, 12, 10, 8, 6 or 5 mg/mL of one or more gentisate compounds in a composition.

One or more ascorbate compounds are suitably present in a pharmaceutical composition in an amount of at least 3 mg/mL, including at least 5, 10, 15, 20, 25, 30, 35, 40, 45, 50, 55 or 60 mg/mL or more.

In certain aspects, the radiopharmaceutical compound does not comprise a prostate-specific membrane antigen (PSMA)-ligand. In other aspects, the radiopharmaceutical compound docs comprise a PSMA-ligand.

In certain aspects, the radiopharmaceutical compound comprises a moiety that can exhibit binding affinity for human serum albumin (HSA).

In certain aspects, preferred radiopharmaceutical compounds may comprise an iodo-phenyl moiety, including 2-iodo-phenyl, 3-iodo-phenyl and/or 4-iodo-phenyl. In addition to iodo, the phenyl may have one or more additional substituents such as alkyl e.g. C₁₋₆ alkyl including methyl, ethyl, propyl, butyl pentyl or hexyl, or other halogen such as F, Cl, Br or I, or other substituent. In certain aspects, phenyl having a single substituent of iodo is preferred.

In certain aspects, preferred radiopharmaceutical compounds may comprise a para-iodo-phenyl moiety including 4-(p-iodophenyl) butyryl.

In other aspects, preferred radiopharmaceutical compounds may comprise a bromo-phenyl moiety, including 2-bromo-phenyl, 3-bromo-phenyl and/or 4-bromo-phenyl. In addition to bromo, the phenyl may have one or more additional substituents such as alkyl e.g. C₁₋₆ alkyl including methyl, ethyl, propyl, butyl, pentyl or hexyl, or halogen such as F, Cl, Br or I, or other substituent. In certain aspects, phenyl having a single substituent of bromo may be preferred.

We have found that including a gentisate compound such as gentisic acid in a radionucleotide/PSMA I & T complex final formulation (i.e., after the incorporation reaction is complete) can suppress formation of impurities. In particular, we have found that the occurrence or amount of a de-iodinated impurity can be suppressed by including a gentisate compound in a radionucleotide/PSMA I & T formulation. This impurity is believed to be a de-iodo form of EuK-Sub-kf-iodo-y-DOTAGA, i.e. an impurity that results from loss of iodine from the 3-iodotyrosine moiety of EuK-Sub-kf-iodo-y-DOTAGA.

Preferred compositions maintain a radiochemical purity of 95% or greater as determined by radio-HPLC after 5 days at 30° C.

A pharmaceutical composition or formulation can be assessed for an at least substantial absence of a de-iodo impurity suitably by a number of methods including spectroscopy or chromatography, particularly radio-HPLC and/or mass spectroscopy.

For instance, a preferred composition may be assessed to have a de-iodo impurity present in an amount of less than a targeted amount such as less than 6.0%, 5.5%, 5.0%, 4.5% 4.0%, 3.5%, 3.0%, 2.5%, 2.0%, 1.5%, 1.0% or 0.5% as determined e.g. by radio-HPLC and/or mass spectroscopy at any time including 1, 2, 3, 4, 5, 6 or more days following preparation of the composition, with the composition being maintained at a particular temperature such as 30° C. In certain aspects, the de-iodination impurity is present in an amount of 4.0% 3.8%, 3.6%, 3.4%, 3.2%, 3.0%, 2.8%, 2.6%, 2.4%, 2.2%, 2.0%, 1.0% or 0.5% or less as e.g. determined by radio-HPLC and/or mass spectroscopy after 5 days at 30° C. Such % values of a dehalogenation impurity or deiodination impurity as referred to herein in the case of HPLC including radio-HPLC analysis can reference the % of the total area of the chromatograph (also may be referred to as radiochemical purity). Such % values of a dehalogenation impurity or deiodination impurity as referred to herein also may be weight % based on total weight of the radiopharmaceutical compounds.

In certain preferred compositions, a de-iodination impurity does not substantially increase in relative amount over a 1, 2, 3, 4, 5 or 6 day period following preparation. For instance, the de-iodination impurity preferably increases in amount no more than 5.0 fold, 4.5 fold, 4-fold, 3.5-fold, 3.0-fold, 2.5-fold, 2.0-fold, 1.5-fold or 1.0-fold determined by radio-HPLC after 5 days at 30° C.

In one aspect, a composition will have considered to have at least a substantial absence of a de-iodination or other identified impurity where the composition has less than 6.0% of the impurity after 3 days at 30° C., for example as measured by radio-HPLC. Preferably, the composition has less than 5.0%, 4.0%, 3.0%, 2.5% or 2.0% of a de-iodination or other identified impurity after 3 days at 30° C., for example as measured by chromatography including radio-HPLC, or mass spectroscopy, or other protocol.

Methods are also provided, including methods of treatment that comprise: a) preparing or providing a radiopharmaceutical compound or pharmaceutical composition or formulation as disclosed herein; and b) assessing the compound or pharmaceutical composition or formulation for the presence of a dehalogenation impurity. The assessed compound or pharmaceutical composition or formulation may be administered to a subject, such as a subject suffering from cancer. The compound or pharmaceutical composition or formulation may be administered may be administered for example after being determined to have a de-iodo impurity present in an amount of less than a targeted amount such as less than 6.0%, 5.5%, 5.0%, 4.5% 4.0%, 3.5%, 3.0%, 2.5%, 2.0%, 1.5%, 1.0% or 0.5% as determined by radio-HPLC and/or mass spectroscopy at any time including 1, 2, 3, 4, 5, 6 or more days following preparation of the composition, with the composition being maintained at a particular temperature such as 30° C. In certain aspects, the de-iodination impurity is present in an amount of 4.0% 3.8%, 3.6%, 3.4%, 3.2%, 3.0%, 2.8%, 2.6%, 2.4%, 2.2%, 2.0%, 1.0% or 0.5% or less as determined e.g. by radio-HPLC and/or mass spectroscopy after 5 days at 30° C.

Suitably, a radiopharmaceutical compound may be present in an aqueous formulation. As understood, a radiochemical impurity will contain a radionuclide e.g. lutetium-177 or a degradation species thereof, while a chemical impurity may or may not contain a radionuclide such as lutetium-177 or a degradation species thereof. Amounts of radiochemical and chemical impurities as referred to herein can be assessed by chromatography or spectroscopy including HPLC, particularly including HPLC radiometric detection (radio-HPLC), or mass spectroscopy, or other techniques.

In a still further aspects, a radiopharmaceutical compound particularly is provided as obtainable by or obtained from a process disclosed herein, including a process for the incorporation radionuclide such as Lutetium-177, Copper-67, Lead-212, Terbium-161 and/or Actinium-225 and a precursor agent such as EuK-Sub-kf-iodo-y-DOTAGA comprising at least one of the following 1), 2) or 3) and preferably at least 1) and 2) and more preferably each of the following 1), 2) and 3):
1) inclusion of one or more ascorbate compound in the Lutetium-177, Copper-67, Lead-212, Terbium-161 and/or Actinium-225 or other radionuclide incorporation reaction mixture. Optionally one or more gentisate compounds also may be present in the radionuclide (such as Lutetium-177, Copper-67, Lead-212, Terbium-161 and/or Actinium-225) incorporation reaction mixture, but such inclusion of gentisate compound is less preferred as discussed. Thus, particularly preferred is inclusion of one or more ascorbate compound in the incorporation reaction mixture and without the inclusion of a gentisate compound;
2) a short heat treatment such up to or less than 30, 20, 15, 12, 10 or 8 minutes as described above; and
3) a post-heating treatment (addition) with one or more gentisate compounds suitably together with and/or one or more ascorbate compounds.

Methods of treatment are also provided including to treat a subject that is suffering from a cell proliferative disease or disorder, particularly a cancer by administering to the subject an effective amount of a radiopharmaceutical compound as disclosed herein including ²¹²Pb-PSMA I & T or other PSMA I & T agents such as ²²⁵Ac-PSMA I & T, ¹⁶¹Tb-PSMA I & T and ⁶⁷Cu-PSMA I & T.

In particular, the present pharmaceutical compositions, including ²¹²Pb-PSMA I & T and/or ²²⁵Ac-PSMA I & T, ¹⁶¹Tb-PSMA I & T and ⁶⁷Cu-PSMA I & T compositions, may be used to treat a subject suffering from prostate cancer, including metastatic castration-resistant prostate cancer (such as may be manifested by progression of the disease despite prior surgical or chemical castration) including those subjects that have progressed following treatment with androgen receptor-axis-targeted (ARAT) therapies.

In one aspect, methods for treating a patient such as a human suffering from cancer, comprising: a) admixing 1) Copper-67, Lutetium-177, Lead-212, Terbium-161, Radium-223 and/or Actinium-225 other or other radioisotope and 2) EuK-Sub-kf-iodo-y-DOTAGA in the substantial or complete absence of a gentisate compound; b) heating the admixed 1) the radioisotope and 2) EuK-Sub-kf-iodo-y-DOTAGA in the substantial or complete absence of a gentisate compound, wherein a complex of the radioisotope and EuK-Sub-kf-iodo-y-DOTAGA is formed; and c) administering the complex of the radioisotope and EuK-Sub-kf-iodo-y-DOTAGA to patient. Suitably, 1) the radioisotope such as Copper-67, Lutetium-177, Lead-212, Terbium-161, Radium-223 and/or Actinium-225, 2) EuK-Sub-kf-iodo-y-DOTAGA and 3) one or more stabilizer compounds are admixed in the absence of a gentisate compound. Suitably, 1)) the radioisotope such as Copper-67, Lutetium-177, Lead-212, Terbium-161, Radium-223 and/or Actinium-225, 2) EuK-Sub-kf-iodo-y-DOTAGA and 3) one or more stabilizer compounds are heated in the absence of a gentisate compound.

In certain aspects, a pharmaceutical formulation comprising a radiopharmaceutical compound comprising ²²⁵Ac is provided, such as ²²⁵Ac-PSMA I & T. In certain aspects, a pharmaceutical formulation comprising a radiopharmaceutical compound comprising ²²⁵Ac may have a volumetric radioactivity at the time of formulation of 0.35 MBq/mL to 15 MBq/mL. In certain aspects, a pharmaceutical formulation comprising a radiopharmaceutical compound comprising ²²⁵Ac may be provided as a single dosage form of a volume of 7 mL to 14 mL, which single dosage form maintains a dose of 5-60 MBq at Day 3 following preparation of the pharmaceutical formulation.

In certain aspects, a pharmaceutical formulation comprising a radiopharmaceutical compound comprising ²¹²Pb is provided. In certain aspects, a pharmaceutical formulation comprising a radiopharmaceutical compound comprising ²¹²Pb may have a volumetric radioactivity at the time of formulation of 3.5 MBq/mL to 32 MBq/mL. In certain aspects, a pharmaceutical formulation comprising a radiopharmaceutical compound comprising ²¹²Pb may be provided as a single dosage form of a volume of 7 mL to 14 mL, which single dosage form maintains a dose of 50-225 MBq at Day 3 following preparation of the pharmaceutical formulation. In certain aspects, the radiopharmaceutical compound comprising ²¹²Pb may comprise a chelating moiety that is TCMC or PSC.

In certain aspects, a pharmaceutical formulation comprising a radiopharmaceutical compound comprising ¹⁶¹Tb is provided. In certain aspects, a pharmaceutical formulation comprising a radiopharmaceutical compound comprising ¹⁶¹Tb may have a volumetric radioactivity at the time of formulation of 70 MBq/mL to 1400 MBq/mL. In certain aspects, a pharmaceutical formulation comprising a radiopharmaceutical compound comprising ¹⁶¹Tb may be provided as a single dosage form of a volume of 7 mL to 14 mL, which single dosage form maintains a dose of 1-10 GB at Day 3 following preparation of the pharmaceutical formulation.

Uses of the present radiopharmaceuticals and pharmaceutical compositions, including complexes of the radioisotopes such as Lutetium-177, Copper-67, Lead-212, Terbium-161 and/or Actinium-225 and EuK-Sub-kf-iodo-y-DOTAGA to treat a patient (such as human) suffering from cancer are also provided. In preferred aspects, the pharmaceutical composition may be obtained or obtainable from a) admixing 1) a radioisotope such as Lutetium-177, Copper-67, Lead-212, Radium-223, Terbium-161 and/or Actinium-225 and 2) such as TCMC, PSC, EuK-Sub-kf-iodo-y-DOTAGA (PSMA I & T) or other complexing agent in the substantial or complete absence of a gentisate compound; and b) heating the admixed 1) radioisotope such Lutetium-177, Copper-67, Lead-212, Radium-223, Terbium-161 and/or Actinium-225 and 2) complexing agent in the substantial or complete absence of a gentisate compound, wherein a complex of the radioisotope and Lutetium-177, Copper-67, Lead-212, Radium-223, Terbium-161 and/or Actinium-225 and the complexing agent such as TCMC, PSC, EuK-Sub-kf-iodo-y-DOTAGA is formed. Suitably, 1) lutetium-177, 2) chelator such as TCMC, PSC, EuK-Sub-kf-iodo-y-DOTAGA or other agent and 3) one or more stabilizer compounds are admixed in the absence of a gentisate compound. Suitably, 1) Lutetium-177, Copper-67, Lead-212, Radium-223, Terbium-161 and/or Actinium-225, 2) EuK-Sub-kf-iodo-y-DOTAGA and 3) one or more stabilizer compounds are heated in the absence of a gentisate compound.

In a further aspect, kits are provided for radiopharmaceutical compounds and composition disclosed herein, including cold kits where the radiopharmaceutical composition can be prepared shortly before administration such as in a medical facility, for example a hospital laboratory or nuclear pharmacy. In such a kit, a complexing compound such as EuK-Sub-kf-iodo-y-DOTAGA or other chelator such as TCMC or PSC may be provided in a vial or other container in lyophilized or other form separate from a radioisotope compound such as Lutetium-177, Copper-67, Lead-212, Radium-223, Terbium-161 and/or Actinium-225. The complexing compound such as TCMC, PSC, EuK-Sub-kf-iodo-y-DOTAGA or other agent and the radioisotope (e.g. Lutetium-177, Copper-67, Lead-212, Radium-223, Terbium-161 and/or Actinium-225) are reacted as disclosed herein at the medical facility to provide the radiopharmaceutical compound which then can be promptly administered to a patient. Such kits including cold kits may comprise components such as, for example, one or more buffering agents such as an acetate compound and/or one or more radioprotectants or stabilizer agents such as ascorbate compound and a gentisate compound.

In a yet further aspect, packaged preparations or products of a radiopharmaceutical compound or pharmaceutical composition as disclosed herein are provided. A packaged preparation may comprise 1) a radiopharmaceutical compound or pharmaceutical composition as disclosed herein and optionally 2) instructions for using the compound or composition for treating a disease particularly cancer such as prostate cancer. Preferably, the packaged preparation will comprise a therapeutically effective amount of the radiopharmaceutical compound or pharmaceutical composition. The instructions suitably may be in written form, including as a packaging label. The radiopharmaceutical compound or pharmaceutical composition suitably may be contained within a lead vessel or other container that is within further packaging that may include product identification, instructions for use or other information.

In certain aspects, a present radiopharmaceutical compound or pharmaceutical composition does not contain a radiopharmaceutical compound that has a PSMA ligand.

In certain aspects, a pharmaceutical composition does not contain a radiopharmaceutical compound that has a EuK-Sub-kf-iodo-y-DOTAGA component.

In certain aspects, a pharmaceutical composition does not contain a radiopharmaceutical compound that comprises ¹⁷⁷Lu.

In certain aspects, pharmaceutical formulations and methods of the present invention do not include ¹⁷⁷Lu-PSMA I & T.

Other aspects of the invention are disclosed infra.

### BRIEF DESCRIPTION OF DRAWINGS

FIG. 1A shows an exemplary reaction with acetate and gentisate (peak at 10.2 min consistent with the gentisate adduct impurity always appears)—20 min reactions at 90° C. min as detailed in Example 1 which follows.

FIGS. 1B-1C show exemplary reactions with ascorbate and gentisate (peak at 10.2 min consistent with the gentisate adduct impurity appears)—20 min reactions at 90° C. as detailed in Example 1 which follows.

FIG. 1D shows an exemplary reaction with just ascorbate (peak at ˜10.2 min consistent with the gentisate adduct impurity never appears)—20 min reactions at 90° C. as detailed in Example 1 which follows.

FIGS. 1E and 1F show HPLC chromatograms of Example 2 which follows.

FIG. 2 shows schematically the general synthesis and formulation of ¹⁷⁷Lu-PSMA I & T in Example 2 which follows.

### DETAILED DESCRIPTION

Applicant notes that an aspect of one of the inventions described herein relates to formulation of radiopharmaceutical agents (radio-ligand targeted therapeutics, or RLTs, in some instances) that include one or more aryl-halide groups where the process focuses on the use and (optionally) avoidance of use of gentisate compounds at different points in the manufacturing process—such as avoidance during heating steps (if any) and/or radioisotope incorporation versus inclusion during the final formulation of the drug product to the radioactive concentration it is intended to be shipped. In the instance of the former it is to prevent the formation of gentisate adducts. In the instance of the latter it is to prevent radiolytic degradation and promote an extended shelf-life which is required to move the setting of “home brew” formulations that has been pervasive in the prior art around RLTs generally, and in doing so finally enabling the centralized GMP manufacture and distribution of a wide range of radiolabeled radiopharmaceutical agents. While not wishing to be bound by any particular theory, Applicant understands that the inclusion of gentisate compounds in the final drug formulation is a means to specifically prevent de-halogenation of halogenated-aryls present in the drug product. As illustrated in the examples, the inclusion of gentisate compounds in final formulation serve purposes, and produces results, which are neither contemplated in the prior art nor obvious from the teachings of those references.

One technical problem with radiopharmaceutical drug products is that the decay of the radionuclide can occur constantly, e.g. during the manufacturing and then during storage of the drug product. The released high energy emissions of the radionuclide decay can induce for example decomposition of water which can lead to chemical modifications including oxidation and/or the cleavage of chemical bonds of the drug product. This is often referred to as radiolysis or radiolytic degradation. The radiolytic degradation of the drug may lead to a decrease in its efficacy, such as reducing its ability to bind to its target receptor, and thereby reduce is ability as a diagnostic and/or therapeutic.

In many countries and jurisdictions, including Europe, Australia, India and other countries, radiopharmaceuticals like ²²⁵Ac-PSMA I & T and ¹⁷⁷Lu-PSMA I & T are regulated as institutional “home-brew” drug formulations, i.e., prepared on site at a single dose scale for immediate use and released for human use with limited quality control testing. Adhering to Current Good Manufacturing Practice (cGMP) principles and achieving a multi-day stabile drug product to enable centralized distribution for outpatient use were not collectively of concern. If shelf-life was determined, it was determined without the use of analytical testing methods that have the sensitivity and/or resolution that would have permitted an understanding and accurate quantification of the type and amount of impurities generated through radiolytic degradation when assessing shelf-life in a manner appropriate for centralized manufacture and broad distribution.

In order to broadly realize the benefit of anti-tumor activity of radiopharmaceuticals that include moieties particularly sensitive to radiolytic degradation—RLTs which include halogenated-aryl groups that are sensitive to radiolytically-mediated dehalogenation—centralized GMP manufacturing with room-temperature stability to radiolytic degradation would be required for shipping to remote locations for use of the radiopharmaceutical several days after it is made.

In that regard, one aspect of one of the inventions described herein focuses on final formulations in which the radioactive concentration (RAC) is reduced after the radioistotope incorporation reaction i.e., to the Shelf-life RAC, and the final formulations includes at least one gentisate compounds (preferably at high concentration) to prevent radiolytic degradation of those moieties and thereby promote an extended shelf-life—i.e., which is required to move from the setting of prior “home brew” formulations and thereby finally enable the centralized GMP manufacture and distribution of such RLTs.

Another aspect of the invention relates to the use of use of significant concentrations of two different stabilizers in the final formulation at the shelf-life RAC-one of which is a triprotic acid (such as a gentisate compounds) and one of which is a diprotic acid (such as an ascorbate compounds).

At the same time that the present application teaches the use of gentisate compounds, such as gentisic acid, in the final formulation, the in certain instances the present application also teaches that gentisate compounds should not be included in an earlier radioisotope incorporation reaction used to form the of radiolabeled RLTs as that can result in the unexpected generation of gentisate adduct impurities.

Overall, the pharmaceutical preparations of the instant invention have properties that are materially different from those previously produced. Due to the high stability (3 or more days at 20° C., 25° C. or even 30° C.) the present invention allows centralized pharmaceutical production at highest quality standards (e.g. cGMP) permitting (i) centralized manufacturing with the ability to ship to clinical treatment sites located considerable distances away and still have pharmaceutical preparations suitable for use in human patients even several days after the incorporated product is generated, and (ii) being able to manufacture the radiopharmaceutical at industrial scale, e.g. batch size which provides the drug product in numerous dose units.

The radiolysis of water generates a complex mixture of reactive intermediates including radicals, highly reactive electrons, ions, and reactive neutral species, collective referred to here as “reactive species”. While some generalizations can be made with respect to how a drug product will react with these “reactive species” based on functional groups and known chemistry, the mixture of drug product degradants generated is typically complex and there is a high degree of uncertainty as to which degradants are formed.

The disclosure provides, inter alia, compounds being used radiopharmaceutical compounds in pharmaceutical compositions.

In one aspect, pharmaceutical compositions are provided that comprise 1) a radiopharmaceutical compound that comprises one or more aryl-halide moieties; and 2) one or more gentisate compounds, wherein the composition maintains a radiochemical purity of 95% or greater as determined by radio-HPLC after 3 days at 30° C., or preferably a radiochemical purity of 95% or greater as determined by radio-HPLC after 5 days at 30° C.

Suitably, the radiopharmaceutical compound comprises one or more of ¹⁷⁷Lu, ²²⁵Ac, ²¹¹At, ²¹²Pb, ¹⁶¹Tb, ²²³Ra or ⁶⁷Cu. In certain aspects, the radiopharmaceutical compound comprises one or more of ¹⁷⁷Lu, ²²⁵Ac, ²¹¹At, ⁶⁷Cu, ²²³Ra, ²⁰³Pb and/or ²¹²Pb. In certain aspects, the radiopharmaceutical compound comprises one or more of ¹⁷⁷Lu, ²²⁵Ac, ⁶⁷Cu, ¹⁶¹Th, ²²³Ra and/or ²¹²Pb.

In preferred aspects, the radiopharmaceutical compound comprises a phenyl moiety having one or more iodo or bromo ring substituents. In certain aspects, a radiopharmaceutical compound that comprises a 4-(p-iodophenyl) butyryl moiety may be preferred.

In certain aspects, preferred radiopharmaceutical compounds may comprise an iodo-phenyl moiety, including 2-iodo-phenyl, 3-iodo-phenyl and/or 4-iodo-phenyl. In addition to iodo, the phenyl may have one or more additional substituents such as alkyl e.g. C₁₋₆ alkyl including methyl, ethyl, propyl, butyl pentyl or hexyl, or other halogen such as F, Cl, Br or I, or other substituent. In certain aspects, phenyl having a single substituent of iodo is preferred.

In other aspects, preferred radiopharmaceutical compounds may comprise a bromo-phenyl moiety, including 2-bromo-phenyl, 3-bromo-phenyl and/or 4-bromo-phenyl. In addition to bromo, the phenyl may have one or more additional substituents such as alkyl e.g. C₁₋₆ alkyl including methyl, ethyl, propyl, butyl, pentyl or hexyl, or halogen such as F, Cl, Br or I, or other substituent. In certain aspects, phenyl having a single substituent of bromo may be preferred.

In certain aspects, the radiopharmaceutical compound may comprise a PSMA-ligand. In other aspects, the radiopharmaceutical compound does not comprise a PSMA-ligand. In additional aspects, the radiopharmaceutical compound may comprise a moiety exhibits binding affinity for human serum albumin (HSA).

In additional aspects, pharmaceutical compositions are also provided that comprise: 1) a radiopharmaceutical compound that comprises i) one or more aryl-halide moieties and ii) one or more of ⁶⁷Cu, ¹⁷⁷Lu, ²²⁵Ac, ²¹²Pb and ²²³Ra; and 2) one or more gentisate compounds.

In a further aspect, pharmaceutical compositions are provided that comprise 1) a radiopharmaceutical compound that comprises one or more aryl-halide moieties and does not contain a PSMA-ligand; and 2) one or more gentisate compounds.

In a further aspect, pharmaceutical compositions are provided that comprise 1) a radiopharmaceutical compound that comprises one or more aryl-halide moieties and does not contain a EuK-Sub-kf-iodo-y-DOTAGA component; and 2) one or more gentisate compounds.

In a further aspect, pharmaceutical compositions are provided that comprise 1) a radiopharmaceutical compound that comprises a 4-(p-iodophenyl) butyryl moiety; and 2) one or more gentisate compounds.

As discussed, preferred compositions maintain a radiochemical purity of 95% or greater as determined by radio-HPLC after 3 days at 30° C., or a radiochemical purity of 95% or greater as determined by radio-HPLC after 5 days at 30° C.

As also discussed, preferred pharmaceutical compositions may further comprise one or more ascorbate compounds in addition to one or more gentisate compounds.

In one aspect, pharmaceutical compositions are provided that comprise: (a) a radiopharmaceutical compound including a complex of radionucleotide (such as ¹⁷⁷Lu, ²²⁵Ac, ²⁰³Pb, ²¹²Pb, 161Tb, ²²³Ra or ⁶⁷Cu) and PSMA I & T (EuK-Sub-kf-iodo-y-DOTAGA); and (b) one or more gentisate compounds, and the composition has a substantial absence of a de-iodo impurity. Preferred compositions also will be at least substantially free of a gentisate adduct impurity. Preferably, the pharmaceutical composition further comprises one or more ascorbate compounds.

Preferably, in the present pharmaceutical compositions, one or more gentisate compound and if present one or more ascorbate compounds are present in an effective amount, for example to provide stability and purity levels as disclosed herein, such as where a composition maintained at 30° C. or less desired purity levels are exhibited for 3, 4 or 5 days or more following preparation of the radiopharmaceutical compound. The exemplary gentisate compound and ascorbate compound amounts disclosed herein are preferred in certain aspects.

In one aspect, preferred radiopharmaceutical agents include a compound having a structure of the following Formula (X):

Ch-L-TM (X)
wherein
   Ch represents a chelating moiety,
   TM represents a target moiety, and
   L comprises a linker including one or more aryl-halide moieties (i.e., an aryl group substituted with at least one halogen (e.g., —Cl, —I, —Br, —F, —At, or isotope thereof));
wherein the compound has a radioisotope (e.g., metal such as ¹⁷⁷Lu, ²²⁵Ac, ²¹¹At and/or ⁶⁷Cu, including metal ions) chelated therewith through the chelating moiety.

In certain embodiments, radioisotope (also referred to in the art as a radionuclide) can be selected such that its decay chain releases one or more of alpha radiation, beta radiation, gamma radiation, Auger electrons, and/or x-rays.

The radioactive isotope of the present invention can be selected to enable imaging and/or radiotherapy. The radioactive isotopes of the present invention may include radioactive metal or semi-metal isotopes. Preferably, the radioactive isotopes are water soluble metal cations.

Exemplary radioisotopes include ¹⁸F, ⁴³K, ⁴⁷Sc, ⁵¹Cr, ⁵⁷Co, ⁵⁸Co, ⁵⁹Fe, ⁶⁷Cu, ⁶⁷Ga, ⁷¹Ge, ⁷²As, ⁷²Se, ⁷⁵Br, ⁷⁶Br, ⁷⁷As, ⁷⁷Br, ⁸¹Rb, ⁸⁸Y, ⁹⁰Y, ⁹⁷Ru, ^{99m}Tc, ¹⁰⁰Pd, ^{101m}Rh, ¹⁰³Pb, ¹⁰⁵Rh, ¹⁰⁹Pd, ¹¹¹Ag, ¹¹¹In, ¹¹³In, ¹¹⁹Sb ¹²¹Sn, ¹²³I, ¹²⁴I, ¹²⁵I, ¹²⁷Cs, ¹²⁸Ba, ¹²⁹Cs, ¹³¹Cs, ¹³¹I, ¹³⁹La, ¹⁴⁰La, ¹⁴²Pr, ¹⁴³Pr, ¹⁴⁹Pm, ¹⁵¹Eu, ¹⁵³Eu, ¹⁵³Sm, ¹⁵⁹Gr, ¹⁶¹Tb, ¹⁶⁵Dy, ¹⁶⁶Ho, ¹⁶⁹Eu, ¹⁷⁵Yb, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁸Au, ¹⁹⁹Ag, ¹⁹⁹Au, ²⁰¹Tl, ²⁰³Pb, ²¹¹At, ²¹²Bi, ²¹²Pb, ²¹³Bi, ²²⁵Ac and ²²⁷Th.

In certain embodiments, the radioisotope is ⁶⁷Cu, ¹⁷⁷Lu, ²¹²Bi, ¹⁶¹Tb, ²¹²Pb, ²²³Ra or ²²⁵Ac.

The chelating moiety can comprise any chelator known in the art that is suitable for chelating the particular radioisotope, see, e.g., Parus et al., “Chemistry and bifunctional chelating agents for binding (177) Lu,” Curr Radiopharm. 2015; 8(2):86-94; Wangler et al., “Chelating agents and their use in radiopharmaceutical sciences,” Mini Rev Med Chem. 2011 October; 11(11):968-83; Liu, “Bifunctional Coupling Agents for Radiolabeling of Biomolecules and Target-Specific Delivery of Metallic Radionuclides,” Adv Drug Deliv Rev. 2008 September; 60(12): 1347-1370. Illustrative examples include, for example, in Table 1.

TABLE 1
Exemplary Chelator Moieties

| Chelator | Structure | (Ch-) in Formula |
|---|---|---|
| DOTA | 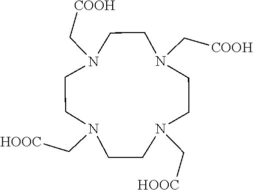 [SMILES: O=C(O)CN1CCN(CC(=O)O)CCN(CC(=O)O)CCN(CC(=O)O)CC1] | 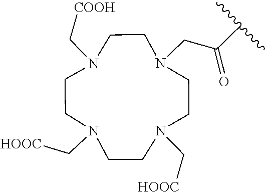 [SMILES: *C(=O)CN1CCN(CC(=O)O)CCN(CC(=O)O)CCN(CC(=O)O)CC1] |
| DOTA-NHS | 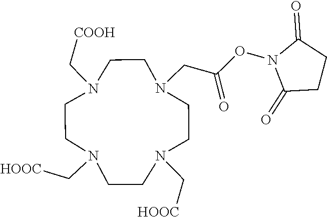 [SMILES: O=C(O)CN1CCN(CC(=O)O)CCN(CC(=O)ON2C(=O)CCC2=O)CCN(CC(=O)O)CC1] | 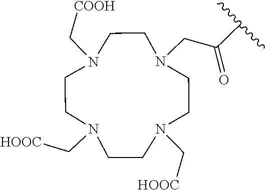 [SMILES: *C(=O)CN1CCN(CC(=O)O)CCN(CC(=O)O)CCN(CC(=O)O)CC1] |
| p-SCN- Bn-NTOA | 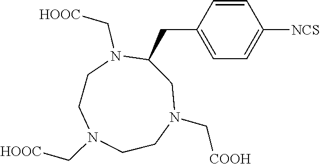 | 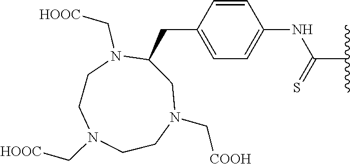 [SMILES: *C(=S)Nc1ccc(C[C@H]2CN(CC(=O)O)CCN(CC(=O)O)CCN2CC(=O)O)cc1] |
| p-SCN- Bn-PCTA | 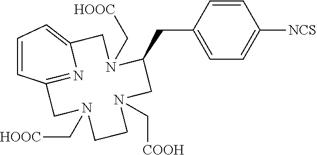 | 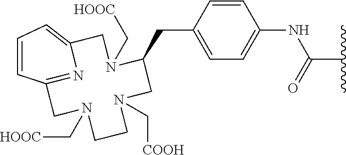 [SMILES: *C(=O)Nc1ccc(C[C@H]2CN(CC(=O)O)CCN(CC(=O)O)Cc3cccc(n3)CN2CC(=O)O)cc1] |
| p-SCN-Bn- Oxo-DO3A | 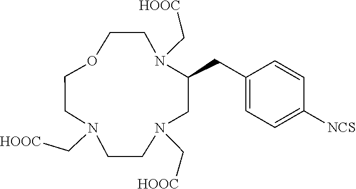 | 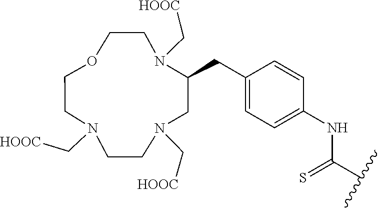 [SMILES: *C(=S)Nc1ccc(C[C@H]2CN(CC(=O)O)CCN(CC(=O)O)CCOCCN2CC(=O)O)cc1] |
| Desferrion- xamine- p-SCN | 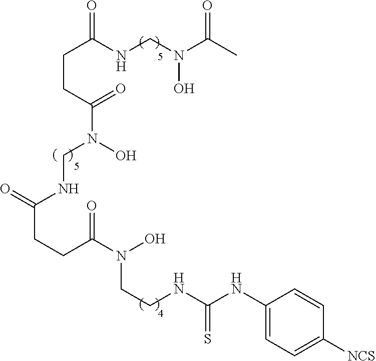 | 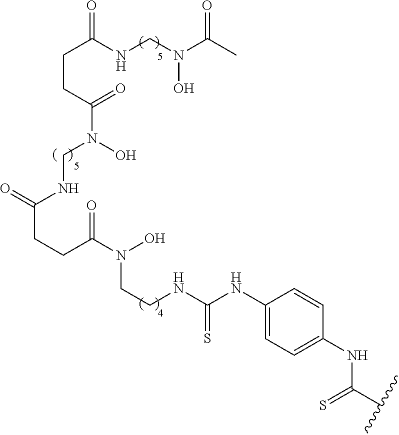 [SMILES: *C(=S)Nc1ccc(NC(=S)NCCN(O)C(=O)CCC(=O)NCN(O)C(=O)CCC(=O)NCN(O)C(C)=O)cc1] |
| Diethylene triamine pentaacetic acid (DTPA) | 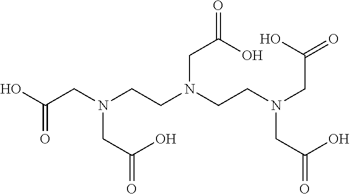 [SMILES: O=C(O)CN(CCN(CC(=O)O)CC(=O)O)CCN(CC(=O)O)CC(=O)O] | 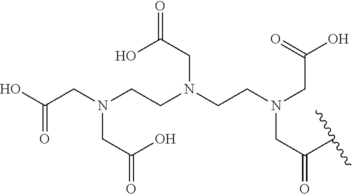 [SMILES: *C(=O)CN(CCN(CCN(CC(=O)O)CC(=O)O)CC(=O)O)CC(=O)O] |
| 1,4,8,11- tetraazacyclo- tetradecane 1,4,8,11- tetraacetic acid (TETA) | 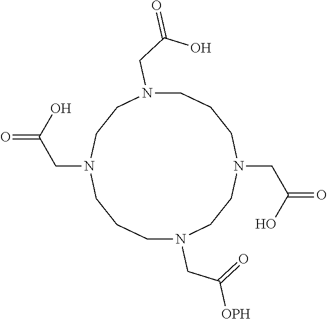 | 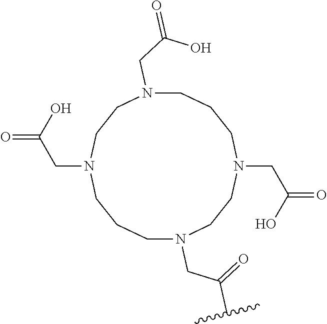 [SMILES: *C(=O)CN1CCCN(CC(=O)O)CCN(CC(=O)O)CCCN(CC(=O)O)CC1] |
| N,N′-Di(2- hydroxybenzyl) etahylene- diamine- N,N′- diacetic acid (HBED) | 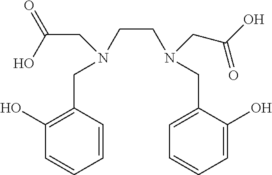 [SMILES: O=C(O)CN(CCN(CC(=O)O)Cc1ccccc1O)Cc1ccccc1O] | 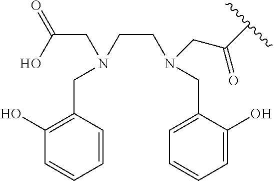 [SMILES: *C(=O)CN(CCN(CC(=O)O)Cc1ccccc1O)Cc1ccccc1O] |
| 4-(4,7-bis(2- tert-butoxy)-2- oxoethyl)-1,4,7- triaza- cyclononan- 1-yl)-5-(tert- butoxyl)-5- oxopentanoic acid (NODAG) | 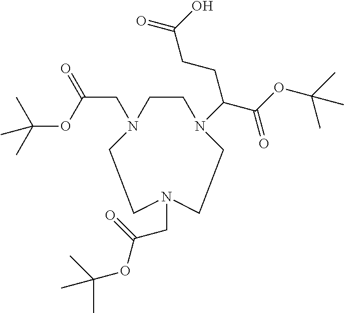 [SMILES: CC(C)(C)OC(=O)CN1CCN(CC(=O)OC(C)(C)C)CCN(C(CCC(=O)O)C(=O)OC(C)(C)C)CC1] | 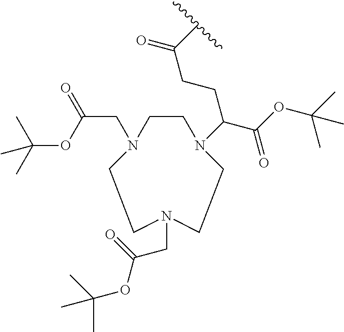 [SMILES: *C(=O)CCC(C(=O)OC(C)(C)C)N1CCN(CC(=O)OC(C)(C)C)CCN(CC(=O)OC(C)(C)C)CC1] |
| 2,2′-(1,4,8,11- tetraazabicyclo [6.6.2] hexadecane- 4,11-diyl) diacetic acid (CB-TE2A) | 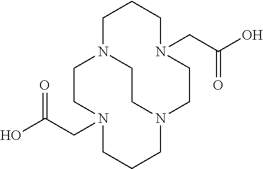 [SMILES: O=C(O)CN1CCCN2CCN(CCCN(CC(=O)O)CC2)CC1] | 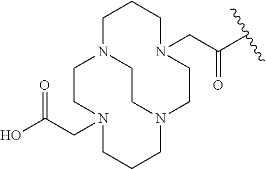 [SMILES: *C(=O)CN1CCCN2CCN(CCCN(CC(=O)O)CC2)CC1] |
| 6-amino-2-(11- (phosphono- methyl)- 1,4,8,11- tetraazabicuclo [6.6.2] hexadecane- 4-yl)hexanoic acid (CB-TE1K1P) | 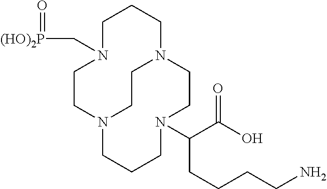 | 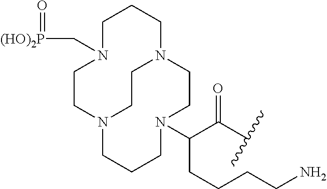 |
| HOPO | 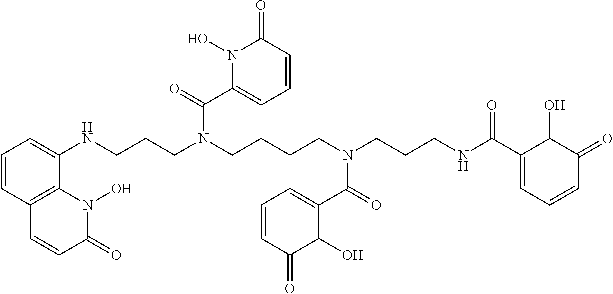 [SMILES: O=C(NCCCN(CCCCN(CCCNc1cccc2ccc(=O)n(O)c12)C(=O)c1cccc(=O)n1O)C(=O)C1=CC=CC(=O)C1O)C1=CC=CC(=O)C1O] | 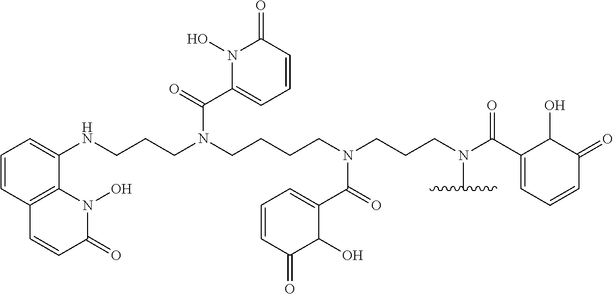 [SMILES: *N(CCCN(CCCCN(CCCNc1cccc2ccc(=O)n(O)c12)C(=O)c1cccc(=O)n1O)C(=O)C1=CC=CC(=O)C1O)C(=O)C1=CC=CC(=O)C1O] |
| DTPA | 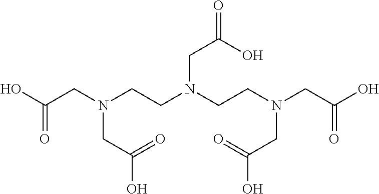 [SMILES: O=C(O)CN(CCN(CC(=O)O)CC(=O)O)CCN(CC(=O)O)CC(=O)O] | 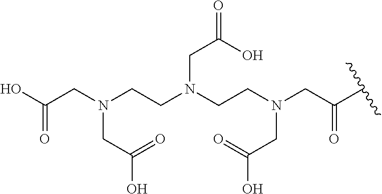 [SMILES: *C(=O)CN(CCN(CCN(CC(=O)O)CC(=O)O)CC(=O)O)CC(=O)O] |
| EDTA | 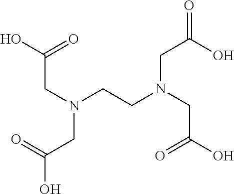 [SMILES: O=C(O)CN(CCN(CC(=O)O)CC(=O)O)CC(=O)O] | 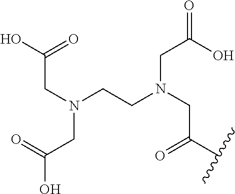 [SMILES: *C(=O)CN(CCN(CC(=O)O)CC(=O)O)CC(=O)O] |
| CHX-A″-DTPA | 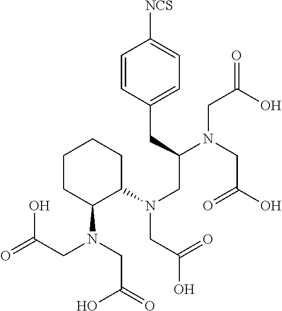 | 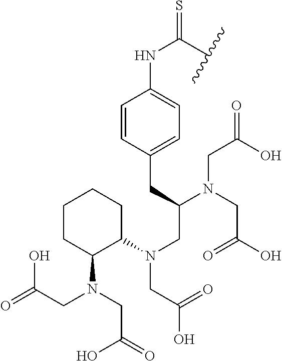 |
| NODASA | 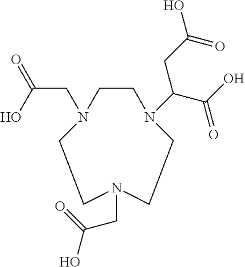 [SMILES: O=C(O)CC(C(=O)O)N1CCN(CC(=O)O)CCN(CC(=O)O)CC1] | 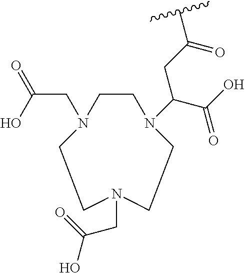 [SMILES: *C(=O)CC(C(=O)O)N1CCN(CC(=O)O)CCN(CC(=O)O)CC1] |
| TCMC | 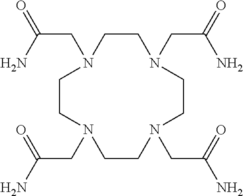 [SMILES: NC(=O)CN1CCN(CC(N)=O)CCN(CC(N)=O)CCN(CC(N)=O)CC1] | 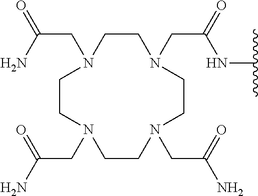 [SMILES: *NC(=O)CN1CCN(CC(N)=O)CCN(CC(N)=O)CCN(CC(N)=O)CC1] |
| TETA | 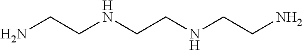 | 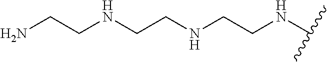 [SMILES: *NCCNCCNCCN] |
| PEPA | 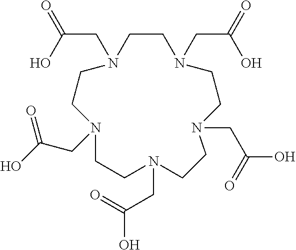 [SMILES: O=C(O)CN1CCN(CC(=O)O)CCN(CC(=O)O)CCN(CC(=O)O)CCN(CC(=O)O)CC1] | 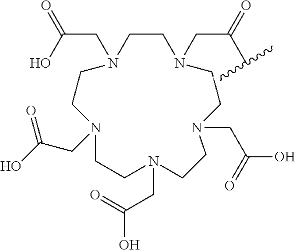 [SMILES: *C(=O)CN1CCN(CC(=O)O)CCN(CC(=O)O)CCN(CC(=O)O)CCN(CC(=O)O)CC1] |
| HEHA | 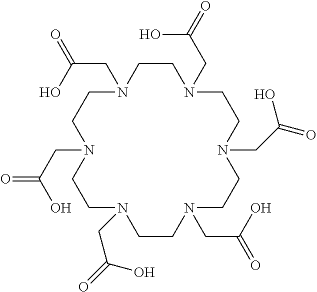 [SMILES: O=C(O)CN1CCN(CC(=O)O)CCN(CC(=O)O)CCN(CC(=O)O)CCN(CC(=O)O)CCN(CC(=O)O)CC1] | 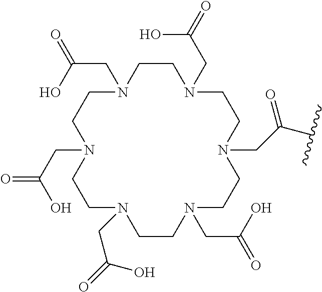 [SMILES: *C(=O)CN1CCN(CC(=O)O)CCN(CC(=O)O)CCN(CC(=O)O)CCN(CC(=O)O)CCN(CC(=O)O)CC1] |
| PSC | 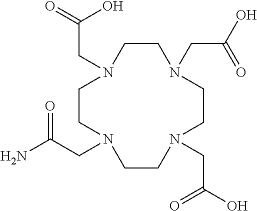 [SMILES: NC(=O)CN1CCN(CC(=O)O)CCN(CC(=O)O)CCN(CC(=O)O)CC1] | 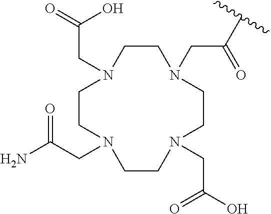 [SMILES: *C(=O)CN1CCN(CC(=O)O)CCN(CC(N)=O)CCN(CC(=O)O)CC1] |

In certain embodiments, the chelator moiety is selected from

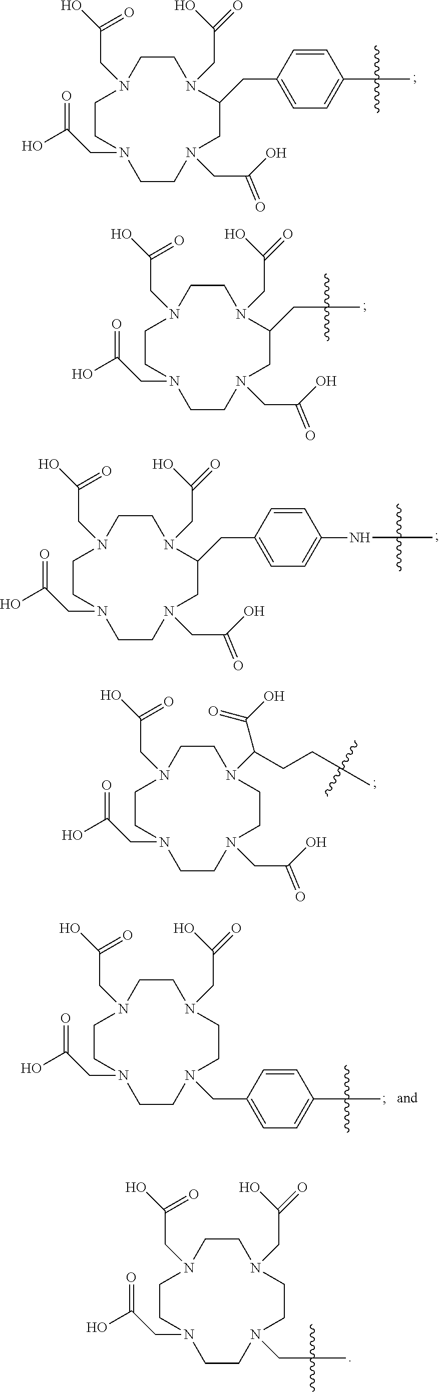

Illustrative examples of metal isotopes and chelators that are suitably form a complex are shown in in Table 2.

TABLE 2

| Isotope | Element | Chelator |
|---|---|---|
| 18 | F | Covalent, or as Al—F - DTPA, NODAGA, NOTA, |
| | | NODA, H3L1, H3L3, H3RESCA, 2-AMPTA, |
| | | NHB-2-AMPDA, 2-AMPDA-HB |
| 47 | Sc | DOTA |
| 64 | Cu | DOTA, NOTA, Cyclam, SAR, CB derivatives |
| 67 | Cu | DOTA, NOTA, Cyclam, SAR, CB derivatives |
| 67 | Ga | DOTA, NOTA, NODAGA, HBED-CC, HBED, |
| | | NOTP, TRAP, THP, DFO, PCTA, DATA, FSC, |
| | | DEDPA |
| 68 | Ga | DOTA, NOTA, NODAGA, HBED-CC, HBED, |
| | | NOTP, TRAP, THP, DFO, PCTA, DATA, FSC, |
| | | DEDPA |
| 89 | Zr | DFO, DFO* (called DFO Star), 4HMS, HOPO |
| 90 | Y | DOTA, DOTAGA, CHX-A″-DTPA, H4octapa |
| 111 | In | NOTA, NODAGA, DOTA, DOTAGA, H(4)octapa |
| 155 | Tb | DOTA |
| 161 | Tb | DOTA |
| 166 | Ho | DO3A-4B4, DOTA, methoxy-DOTA, CHX-A″ |
| | | DTPA |
| 177 | Lu | DOTA, DOTAGA, |
| 212 | Pb | DOTA, TCMC, PSC |
| 213 | Bi | DOTA, DO3A-B, DO3A, PIP-DOTA, DEPA, NPTA, |
| | | NETA, DOTP, PSC, EDTA, DTPA, C-DTPA, |
| | | 1B-DTPA, 1B4M-DTPA, 1M3B-DTPA, |
| | | 1B3M-DTPA, CHX-A″-DTPA, p-SCN-Bn- |
| | | CHX-DTPA, PIP-DTPA, AZEP-DTPA, N53Pa, |
| | | [p-NO2-Bn-neunpa - LOTS of examples |
| | | in the paper reference. |
| 223 | Ra | MACROPA |
| 225 | Ac | DOTA, DOTAGA, HEHA, macropa, |
| 203 | Pb | DOTA, TCMC, PSC |

In certain embodiments, preferred radiopharmaceutical compounds include ²¹²Pb. Exemplary preferred chelator of ²¹²Pb are shown in Table 3.

TABLE 3
Exemplary ²¹²Pb Chelator Moieties

| Chelator | Structure | (Ch-) in Formula |
|---|---|---|
| TCMC (DOTAM) | 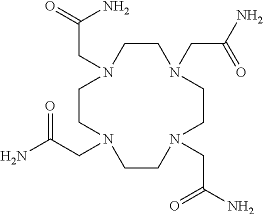 [SMILES: NC(=O)CN1CCN(CC(N)=O)CCN(CC(N)=O)CCN(CC(N)=O)CC1] | 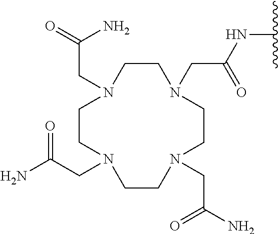 [SMILES: *NC(=O)CN1CCN(CC(N)=O)CCN(CC(N)=O)CCN(CC(N)=O)CC1] |
| p-SCN- Bn- TCMC | 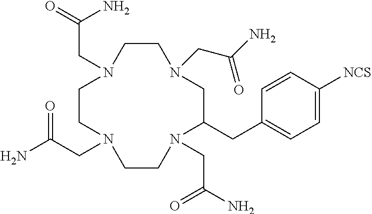 | 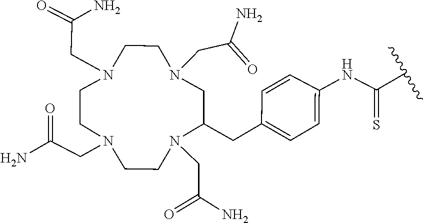 [SMILES: *C(=S)Nc1ccc(CC2CN(CC(N)=O)CCN(CC(N)=O)CCN(CC(N)=O)CCN2CC(N)=O)cc1] |
| PSC | 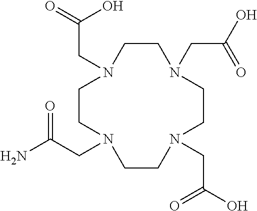 [SMILES: NC(=O)CN1CCN(CC(=O)O)CCN(CC(=O)O)CCN(CC(=O)O)CC1] | 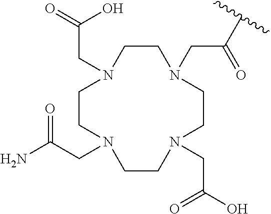 [SMILES: *C(=O)CN1CCN(CC(=O)O)CCN(CC(N)=O)CCN(CC(=O)O)CC1] |

In certain embodiments, preferred radiopharmaceutical compounds include ⁶⁷Cu. Exemplary preferred chelator of ⁶⁷Cu are shown in Table 4.

TABLE 4
Exemplary ⁶⁷Cu Chelator Moieties

| Chelator | Structure | (Ch-) in Formula |
|---|---|---|
| NOTA | 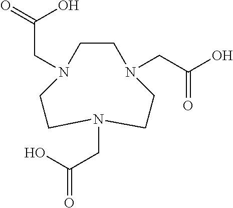 [SMILES: O=C(O)CN1CCN(CC(=O)O)CCN(CC(=O)O)CC1] | 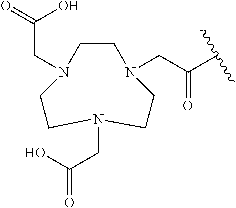 [SMILES: *C(=O)CN1CCN(CC(=O)O)CCN(CC(=O)O)CC1] |
| NOTA- NHS | 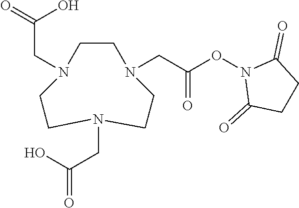 [SMILES: O=C(O)CN1CCN(CC(=O)O)CCN(CC(=O)ON2C(=O)CCC2=O)CC1] | 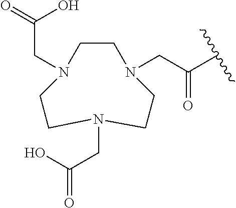 [SMILES: *C(=O)CN1CCN(CC(=O)O)CCN(CC(=O)O)CC1] |
| p-NH₂-Bn- NOTA | 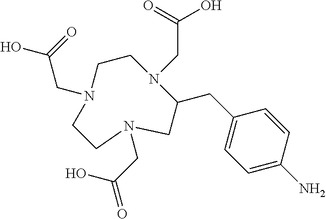 [SMILES: Nc1ccc(CC2CN(CC(=O)O)CCN(CC(=O)O)CCN2CC(=O)O)cc1] | 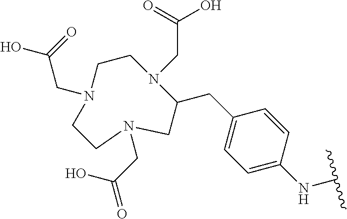 [SMILES: *Nc1ccc(CC2CN(CC(=O)O)CCN(CC(=O)O)CCN2CC(=O)O)cc1] |
| p-SCN-Bn- NOTA | 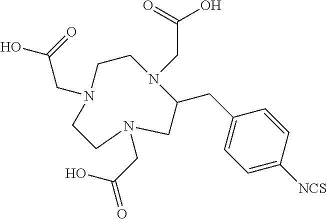 | 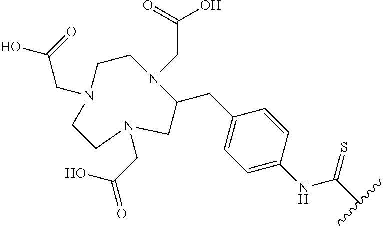 [SMILES: *C(=S)Nc1ccc(CC2CN(CC(=O)O)CCN(CC(=O)O)CCN2CC(=O)O)cc1] |
| Maleimide- NOTA | 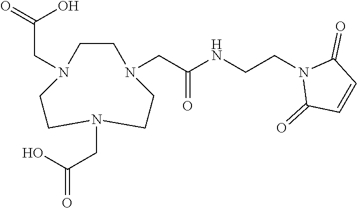 [SMILES: O=C(O)CN1CCN(CC(=O)O)CCN(CC(=O)NCCN2C(=O)C=CC2=O)CC1] | 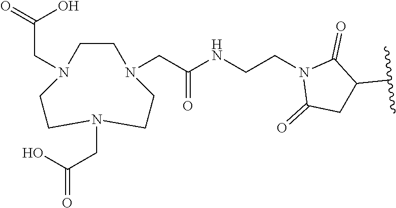 [SMILES: *C1CC(=O)N(CCNC(=O)CN2CCN(CC(=O)O)CCN(CC(=O)O)CC2)C1=O] |
| TETA | 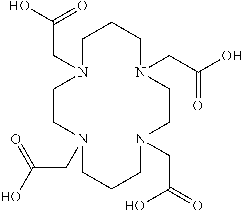 [SMILES: O=C(O)CN1CCCN(CC(=O)O)CCN(CC(=O)O)CCCN(CC(=O)O)CC1] | 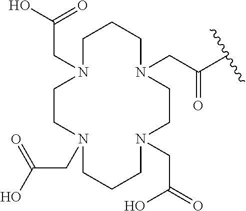 [SMILES: *C(=O)CN1CCCN(CC(=O)O)CCN(CC(=O)O)CCCN(CC(=O)O)CC1] |
| TE2A | 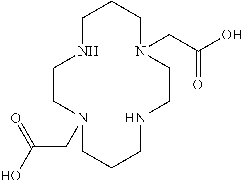 [SMILES: O=C(O)CN1CCCNCCN(CC(=O)O)CCCNCC1] | 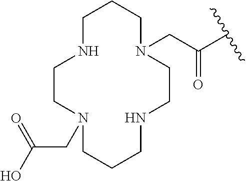 [SMILES: *C(=O)CN1CCCNCCN(CC(=O)O)CCCNCC1] |
| CB-TE2A | 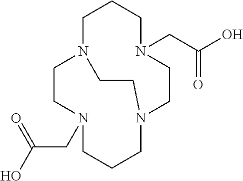 [SMILES: O=C(O)CN1CCCN2CCN(CCCN(CC(=O)O)CC2)CC1] | 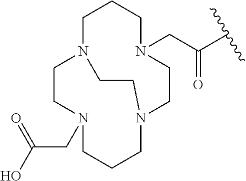 [SMILES: *C(=O)CN1CCCN2CCN(CCCN(CC(=O)O)CC2)CC1] |
| C-Cyclam | 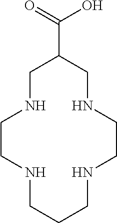 [SMILES: O=C(O)C1CNCCNCCCNCCNC1] | 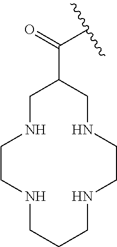 [SMILES: *C(=O)C1CNCCNCCCNCCNC1] |
| CPTA | 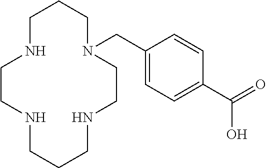 [SMILES: O=C(O)c1ccc(CN2CCCNCCNCCCNCC2)cc1] | 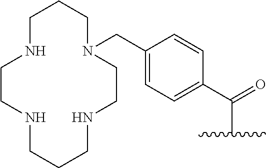 [SMILES: *C(=O)c1ccc(CN2CCCNCCNCCCNCC2)cc1] |
| PCB- TE2A | 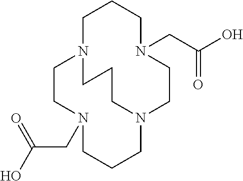 [SMILES: O=C(O)CN1CCCN2CCCN(CCCN(CC(=O)O)CC2)CC1] | 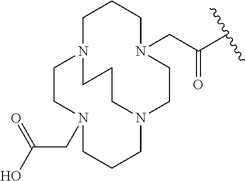 [SMILES: *C(=O)CN1CCCN2CCCN(CCCN(CC(=O)O)CC2)CC1] |
| DiAmSar | 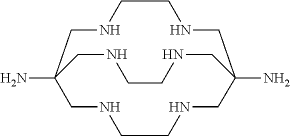 [SMILES: NC12CNCCNCC(N)(CNCCNC1)CNCCNC2] | 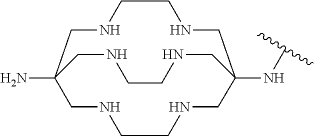 [SMILES: *NC12CNCCNCC(N)(CNCCNC1)CNCCNC2] |
| SarAr | 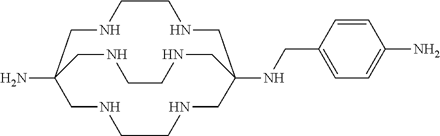 [SMILES: Nc1ccc(CNC23CNCCNCC(N)(CNCCNC2)CNCCNC3)cc1] | 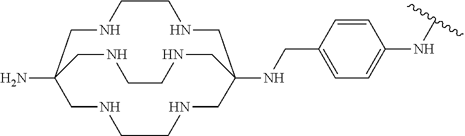 [SMILES: *Nc1ccc(CNC23CNCCNCC(N)(CNCCNC2)CNCCNC3)cc1] |
| SarAr-NCS | 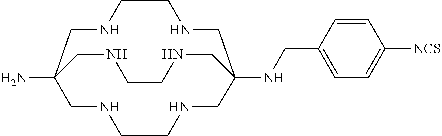 | 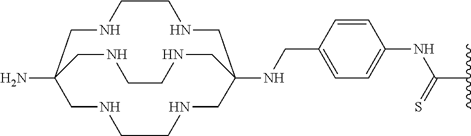 [SMILES: *C(=S)Nc1ccc(CNC23CNCCNCC(N)(CNCCNC2)CNCCNC3)cc1] |
| MeCOSar | 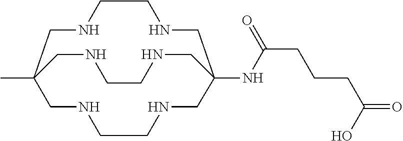 [SMILES: CC12CNCCNCC(NC(=O)CCCC(=O)O)(CNCCNC1)CNCCNC2] | 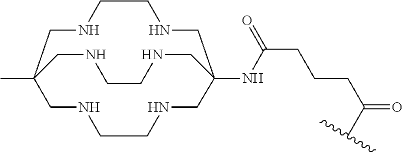 [SMILES: *C(=O)CCCC(=O)NC12CNCCNCC(C)(CNCCNC1)CNCCNC2] |
| MeCOSar- NHS | 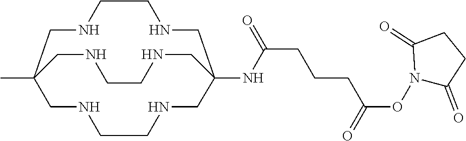 [SMILES: CC12CNCCNCC(NC(=O)CCCC(=O)ON3C(=O)CCC3=O)(CNCCNC1)CNCCNC2] | 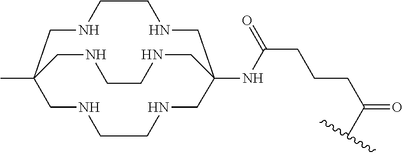 [SMILES: *C(=O)CCCC(=O)NC12CNCCNCC(C)(CNCCNC1)CNCCNC2] |
| BaBaSar | 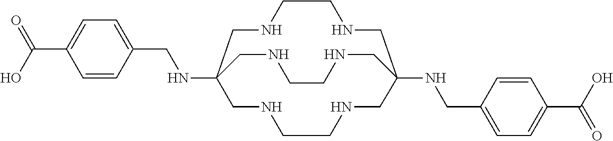 [SMILES: O=C(O)c1ccc(CNC23CNCCNCC(NCc4ccc(C(=O)O)cc4)(CNCCNC2)CNCCNC3)cc1] | 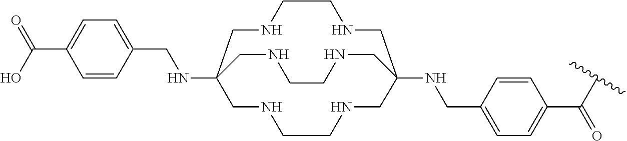 [SMILES: *C(=O)c1ccc(CNC23CNCCNCC(NCc4ccc(C(=O)O)cc4)(CNCCNC2)CNCCNC3)cc1] |

In one aspect, preferred radiopharmaceutical agents include a compound a structure of the following Formula (X-a):

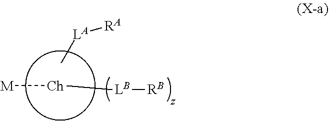

wherein:
M is a metal (e.g. ¹⁷⁷Lu, ²²⁵Ac, 211At and/or ⁶⁷Cu and includes metal ions);
Ch is a ligand that forms a coordination or a chelate with the metal;
R^{A} is an aryl group substituted with at least one halogen (e.g., —Cl, —I, —Br, —F, —At, or isotope thereof);
Each R^{B} is an independent hydrogen or non-hydrogen substituent;
L^{A} is a linker (e.g., a bond, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or combinations thereof);
Each L^{B} is an independent linker (e.g., a bond, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or combinations thereof); and
z is an integer from 0 to 4;
provided that at least one of M and R^{A} comprises radioactive isotope (e.g., radioactive metal ion or radioactive halogen).

In a further aspect, preferred radiopharmaceutical agents include a compound having a structure of the following Formula (X-b):

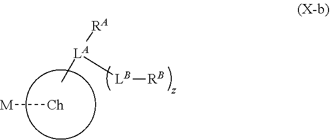

wherein:
M is a metal (e.g. ¹⁷⁷Lu, ²²⁵Ac, ²¹¹At and/or ⁶⁷Cu and includes metal ions);
Ch is a ligand that forms a coordination or a chelate with the metal;
R^{A} is an aryl group substituted with at least one halogen (e.g., —Cl, —I, —Br, —F, —At, or isotope thereof);
Each R^{B} is an independent hydrogen or non-hydrogen substituent;
L^{A} is a linker (e.g., a bond, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or combinations thereof);
Each L^{B} is an independent linker (e.g., a bond, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or combinations thereof); and
z is an integer from 0 to 2;
provided that at least one of M and R^{A} comprises a radioactive isotope (e.g., radioactive metal ion or radioactive halogen).

In certain aspects, in Formula (X-a) or (X-b) above, the radioactive isotope includes one or more of ¹⁷⁷Lu, ²²⁵Ac, ⁶⁷Cu, ¹³¹I, and ¹⁹F. In some embodiments, the compounds does not include ¹⁷⁷Lu. In some embodiments, the compounds of Formula (X-a) or (X-b) include ¹³¹I. In some embodiments, the compounds of Formula (X-a) or (X-b) includes ²²⁵Ac. In some embodiments, the compounds of Formula (X-a) or (X-b) includes ⁶⁷Cu. In some embodiments, the compounds of Formula (X) includes ¹⁹F.

In certain aspects, in Formula (X-a) or (X-b) above, the ligand is a chelate with high affinity or low dissociation to the metal or metal ion (e.g., divalent, or trivalent metal cation). In some embodiments, the chelate may have a macromolecular ring structure that may capture or accommodate the metal ion in the ring structure. In some embodiments, the chelate may not have a macromolecular ring structure. The non-macromolecular chelate may have exposed electron donor atoms or groups (e.g., nitrogen or oxygen) to form a coordinate with the metal or metal ion (e.g., divalent, or trivalent metal cation). Exemplary chelate may include, but not be limited to, 2,2′,2″,2″-(1,4,7,10-Tetraazacyclododecane-1,4,7,10-tetrayl)tetraacetic acid (“DOTA”), 1,4,7,10-tetraazacyclododecane,1-(glutaric acid)-4,7,10-triacetic acid (“DOTA-GA”), N,N′-dipyridoxylethylenediamine-N,N′-diacetate 5,5′-bis(phosphate) (“PLED”), dipyridoxyl diphosphate (“DPDP”) or the like.

In certain aspects, in Formula (X-a) or (X-b), L^{B}-R^{B} may include a peptide (e.g., natural or non-natural peptide having 1-10 amino acids). In some embodiments, the L^{B}-R^{B} may include a moiety including a part or whole biologically active motif. In some embodiments, the L^{B}-R^{B} may include a moiety including a part or whole specific antigen or neoantigen motif (e.g., prostate-specific membrane antigen (PSMA)).

In some embodiments, the compounds of Formula (X-a) or (X-b) may include a moiety including a part or a whole a prostate-specific membrane antigen (PSMA). In some embodiments, the compounds of Formula (X) does not include a moiety including a part or a whole PSMA.

In one aspect, preferred radiopharmaceutical agents include a compound having a structure of the following Formula (I):

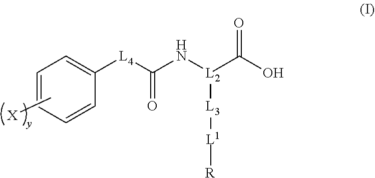

wherein:
X is halogen (e.g., —Cl, —I, —Br, or —F, or isotope thereof);
y is an integer of 1 to 5;
R comprises a chelating moiety (Ch) and a radioactive labeling group or metal (M) (e.g., ¹⁷⁷Lu, ²²⁵Ac, or ⁶⁷Cu, or isotope ions thereof);
L¹ is a linker (e.g., a bond, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or combinations thereof);
L² is a linker (e.g., a bond, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or combinations thereof), which may be divalent or trivalent;
L³ is a linker (e.g., a bond, —NHC(O)—, C(O) NH—, —NH—, or C(O)—); and
L⁴ is a linker (e.g., a bond, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or combinations thereof).

In certain aspects, the compound of Formula (I) has a structure of Formula (I-A), or (I-B),

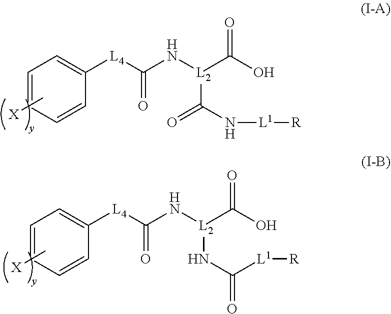

wherein:
L¹ is a substituted or unsubstituted C₁-C₁₂ alkylene, a substituted or unsubstituted 2 to 12 membered heteroalkylene, substituted or unsubstituted C₃-C₆ cycloalkylene, substituted or unsubstituted 5 to 8 membered heterocycloalkylene, substituted or unsubstituted phenyl, substituted or unsubstituted 5 to 8 membered heteroarylene, or combinations thereof;
L² is a substituted or unsubstituted C₁-C₁₂ alkylene, a substituted or unsubstituted 2 to 12 membered heteroalkylene, substituted or unsubstituted C₃-C₆ cycloalkylene, substituted or unsubstituted 5 to 8 membered heterocycloalkylene, substituted or unsubstituted phenyl, substituted or unsubstituted 5 to 8 membered heteroarylene, or combinations thereof; and
L⁴ is a substituted or unsubstituted C₁-C₁₂ allylene.

In some embodiments, the substituents attached to L′ and/or L² may include a peptide (e.g., peptide having 4-20 amino acids), or a macromolecule.
X, y and R are as described above.

In certain embodiments, the compound of Formula (I) has a structure of Formula (I-C) or (I-D),

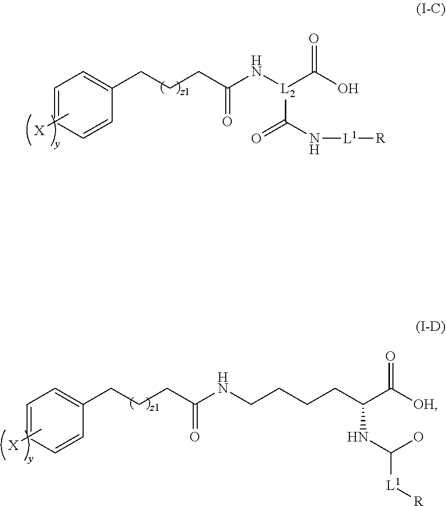

wherein z1 is an integer of 1 to 3. X, y, L¹, L², R are as described above.

In certain aspects, radiopharmaceutical agents of Formula (I′) comprise a compound having structure of Formula (I′)

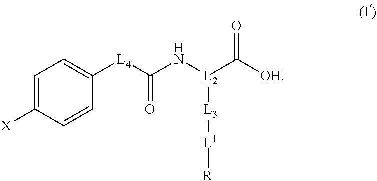

X, L¹, L², L³, L⁴ and R are as described above.

In certain aspects, the compound of Formula (I′) has a structure of Formula (I′-A), or (I′—B),

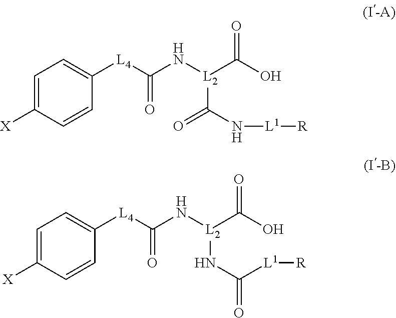

wherein:
L¹ is a substituted or unsubstituted C₁-C₁₂ alkylene, a substituted or unsubstituted 2 to 12 membered heteroalkylene, substituted or unsubstituted C₃-C₆ cycloalkylene, substituted or unsubstituted 5 to 8 membered heterocycloalkylene, substituted or unsubstituted phenyl, substituted or unsubstituted 5 to 8 membered heteroarylene, or combinations thereof;
L² is a substituted or unsubstituted C₁-C₁₂ alkylene, a substituted or unsubstituted 2 to 12 membered heteroalkylene, substituted or unsubstituted C₃-C₆ cycloalkylene, substituted or unsubstituted 5 to 8 membered heterocycloalkylene, substituted or unsubstituted phenyl, substituted or unsubstituted 5 to 8 membered heteroarylene, or combinations thereof; and
L⁴ is a substituted or unsubstituted C₁-C₁₂ allylene.

In some embodiments, the substituents attached to L¹ and/or L² may include a peptide (e.g., peptide having 4-20 amino acids), or a macromolecule.

In certain embodiments, the compounds of Formula (I′) has a structure of Formula (I′—C) or (I′-D),

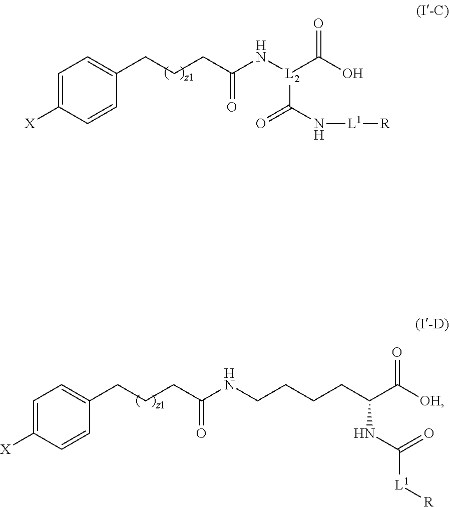

wherein z1 is an integer of 1 to 3. X, L¹, L², R are as described above.

In certain embodiments, X is —I or —Br.

Exemplary compounds of Formula (I) or (I′) may have the following formula:

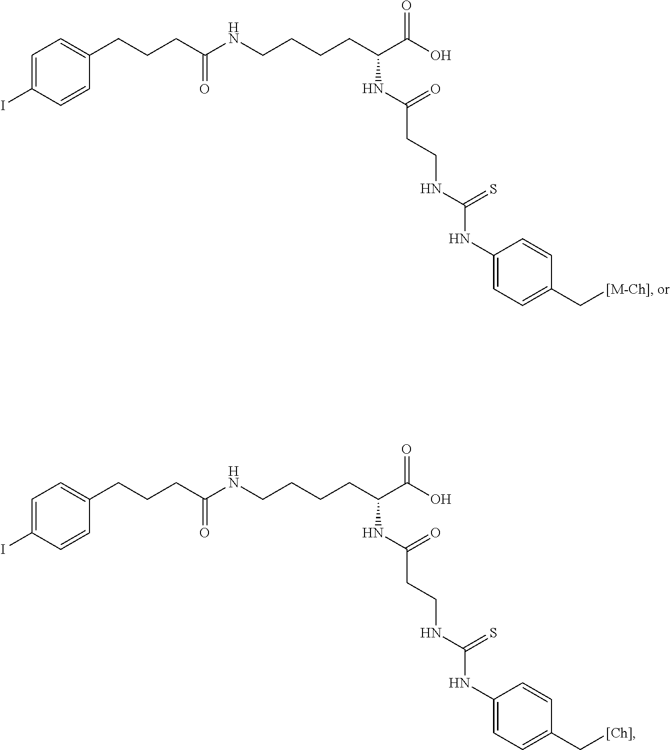

wherein M and Ch are as described above.

Exemplary compounds of Formula (I) or (I′) include the following Compounds A, A′, B and B′

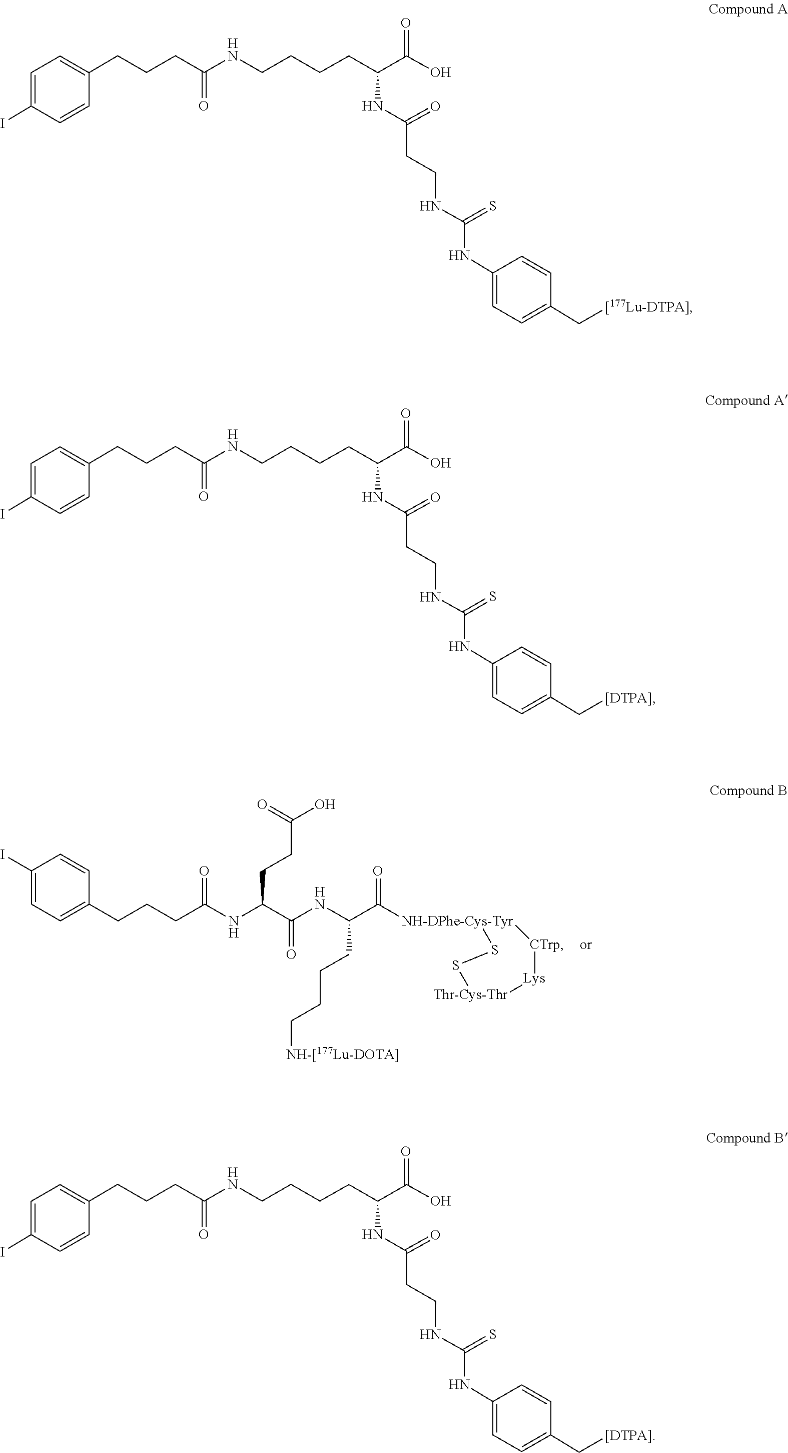

In another preferred aspect, the radiopharmaceutical agents comprise a compound having a structure of the following Formula (II)

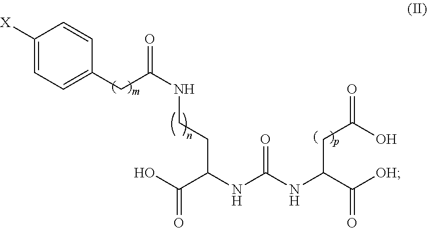

wherein:
X is halogen (e.g., —I, or —Br, or isotope thereof);
n is an integer of 1 to 3;
m is an integer of 1 to 4; and
p is an integer of 1 to 4.

In some embodiments, in Formula (II), p is 1 or 2.

In some embodiments, in Formula (II), n is 2, 3, or 4.

In some embodiments, in Formula (II), m is 1, 2, or 3.

In some embodiments, the compound has the structure of

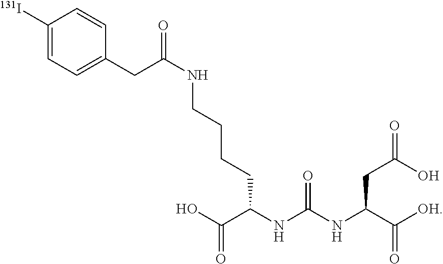 [SMILES: O=C(O)C[C@H](NC(=O)N[C@@H](CCCCNC(=O)Cc1ccc([131I])cc1)C(=O)O)C(=O)O]

In one preferred aspect, the radiopharmaceutical agents comprise a compound having a structure of the following structure of Formula (III),

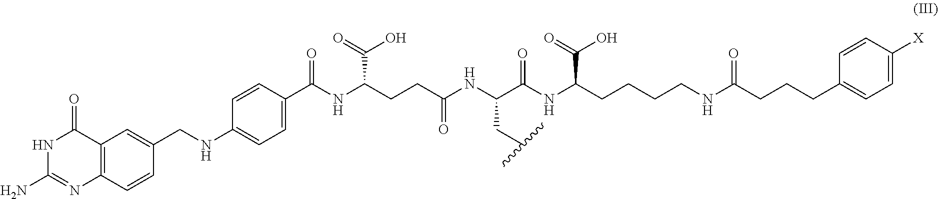

wherein

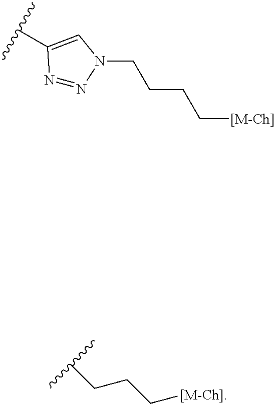

X, M, and Ch are as described above. In some embodiments, Ch is DOTA, and M is ¹⁷⁷Lu or ⁴⁷Sc. In some embodiments, Ch is NODAGA and M is ⁶⁷Cu.

In a preferred aspect, the radiopharmaceutical agents comprise a compound having a structure of the following Formula (IV),

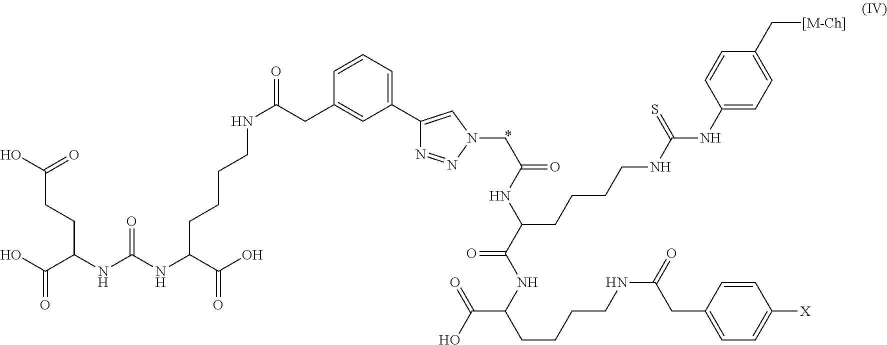

wherein * may optionally include PEG spacers (e.g., PEC₃₋₁₂). X, M, and Ch are as described above.

In one preferred aspect, the radiopharmaceutical agents comprise a compound having a structure of the following Formula (V),

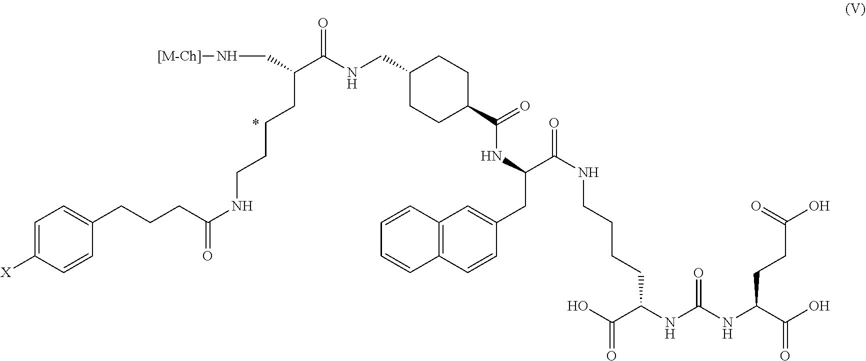

wherein * may optionally include (Asp) q (e.g., q is an integer 2 to 5). X, M, and Ch are as described above.

In one preferred aspect, the radiopharmaceutical agents comprise a compound having a structure of the following Formula (VI):

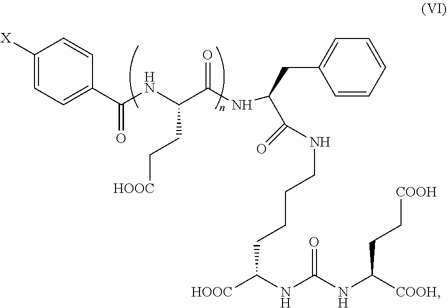

wherein n is an integer of 1 or 5. X is as described above.

In one preferred aspect, the radiopharmaceutical agent comprises one of the following structures of Formula (C) or (C′)

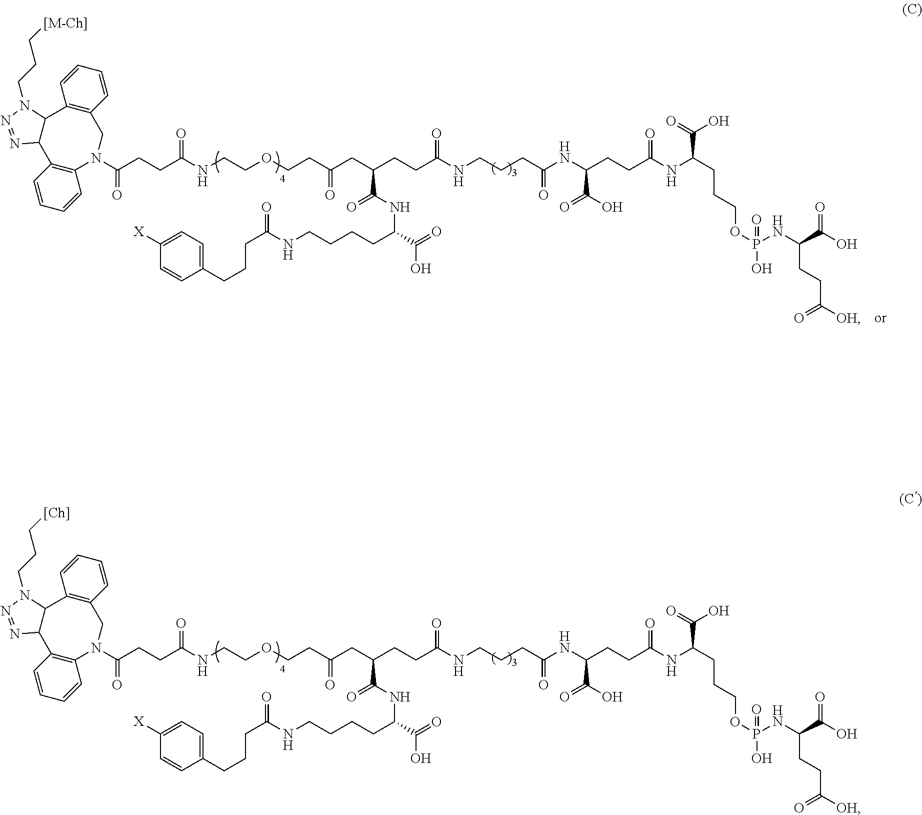

wherein X, M, and Ch are as described above.

Exemplary radiopharmaceutical agent comprises one of the following structures (referred to herein as Compound C, or Compound C′)

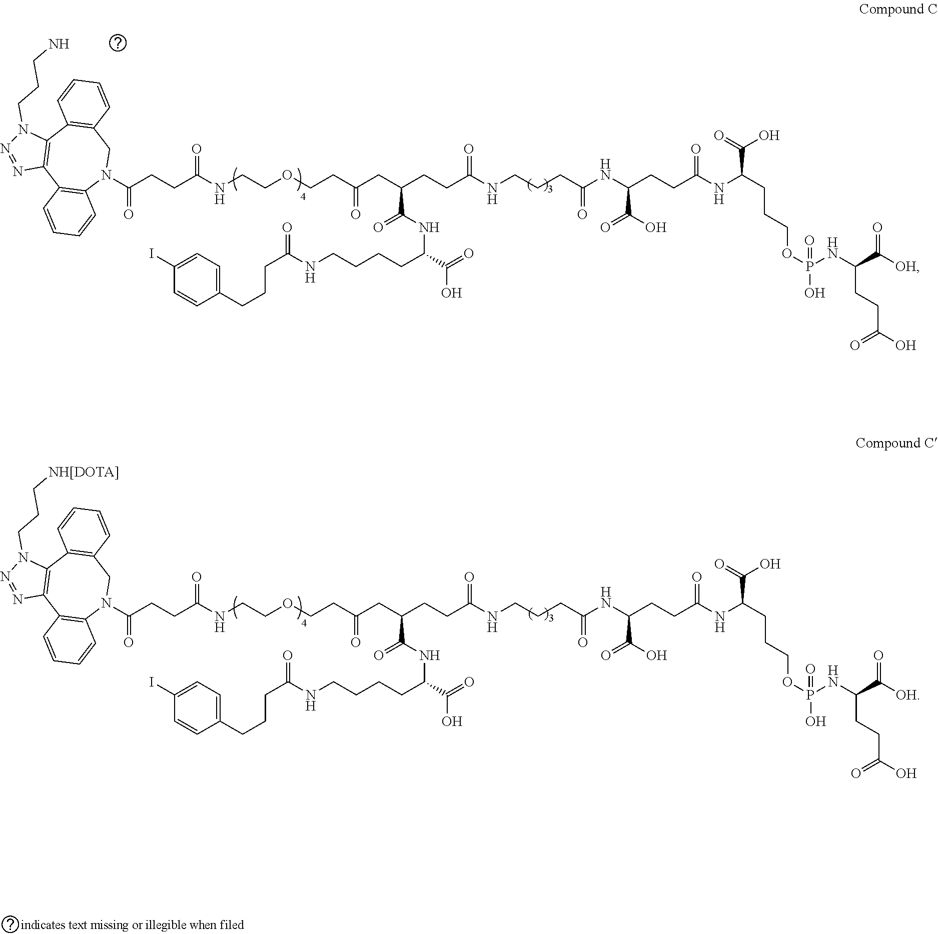

In one preferred aspect, the radiopharmaceutical agent comprises one of the following structures of Formula (D) or (D′):

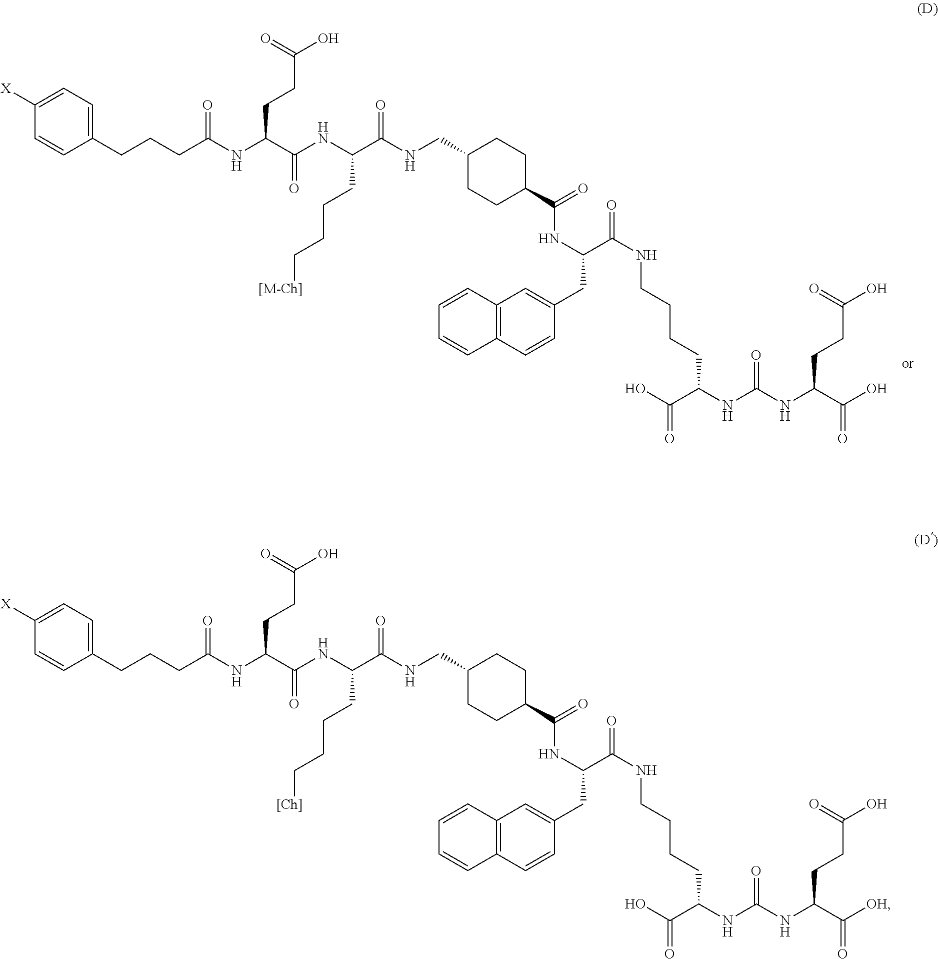

wherein X, M, and Ch are as described above.

### Compound D′.

Exemplary radiopharmaceutical agent comprises one of the following structures (referred to herein as Compound D and D′):

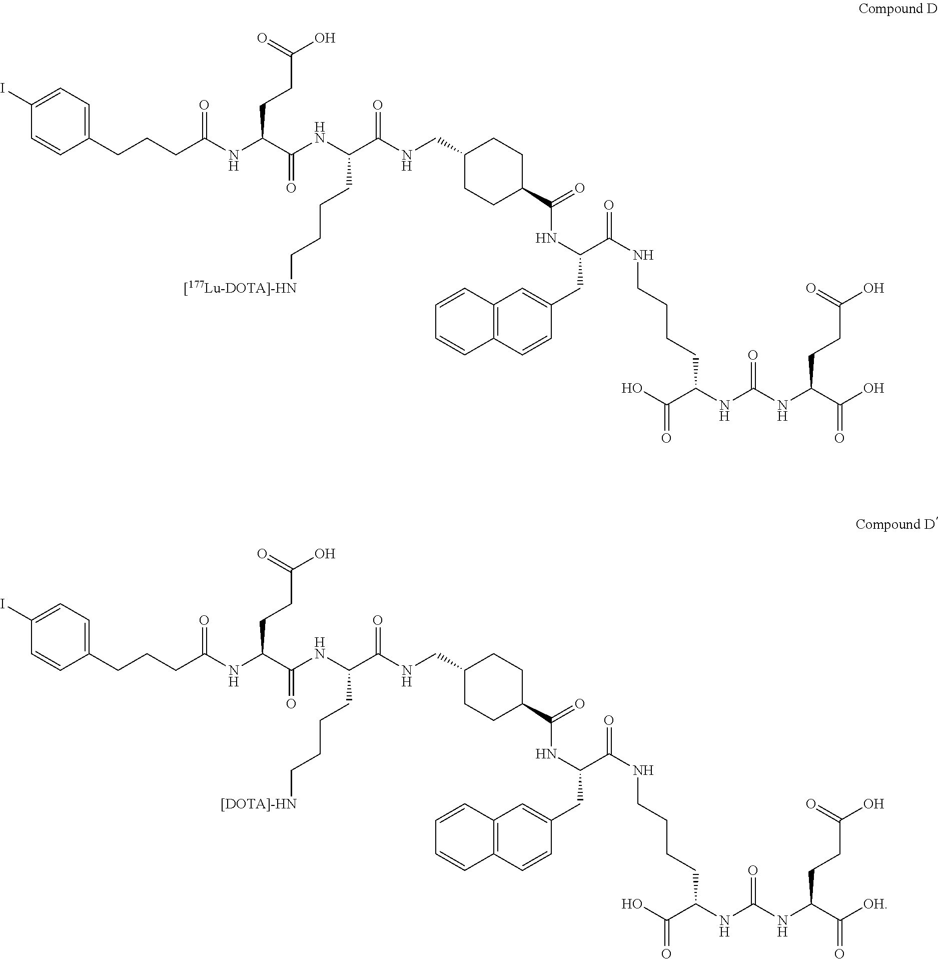

In one preferred aspect, the radiopharmaceutical agent comprises one of the following structures of Formula (E), (E′), (F), (F′), (G) or (G′):

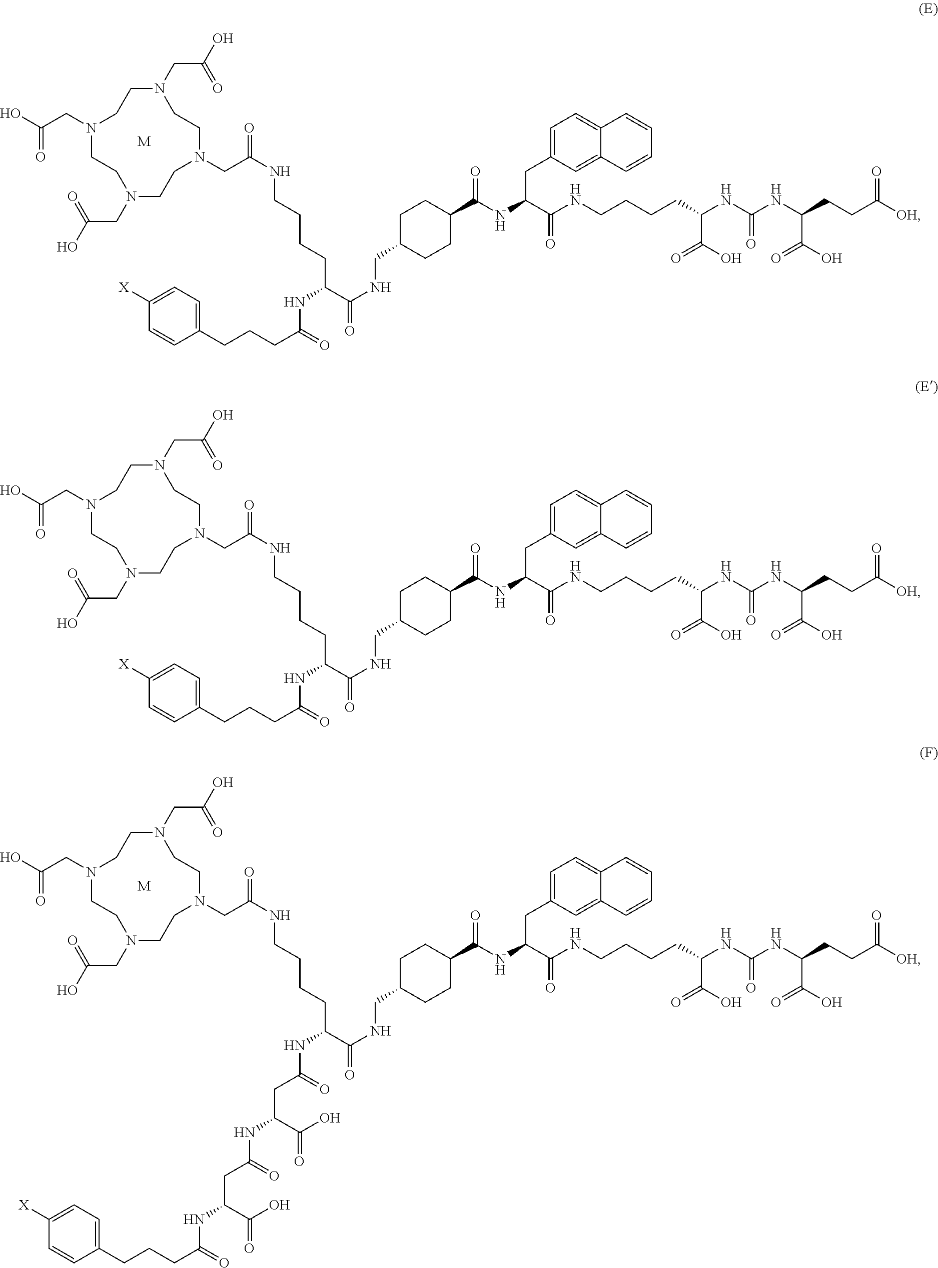

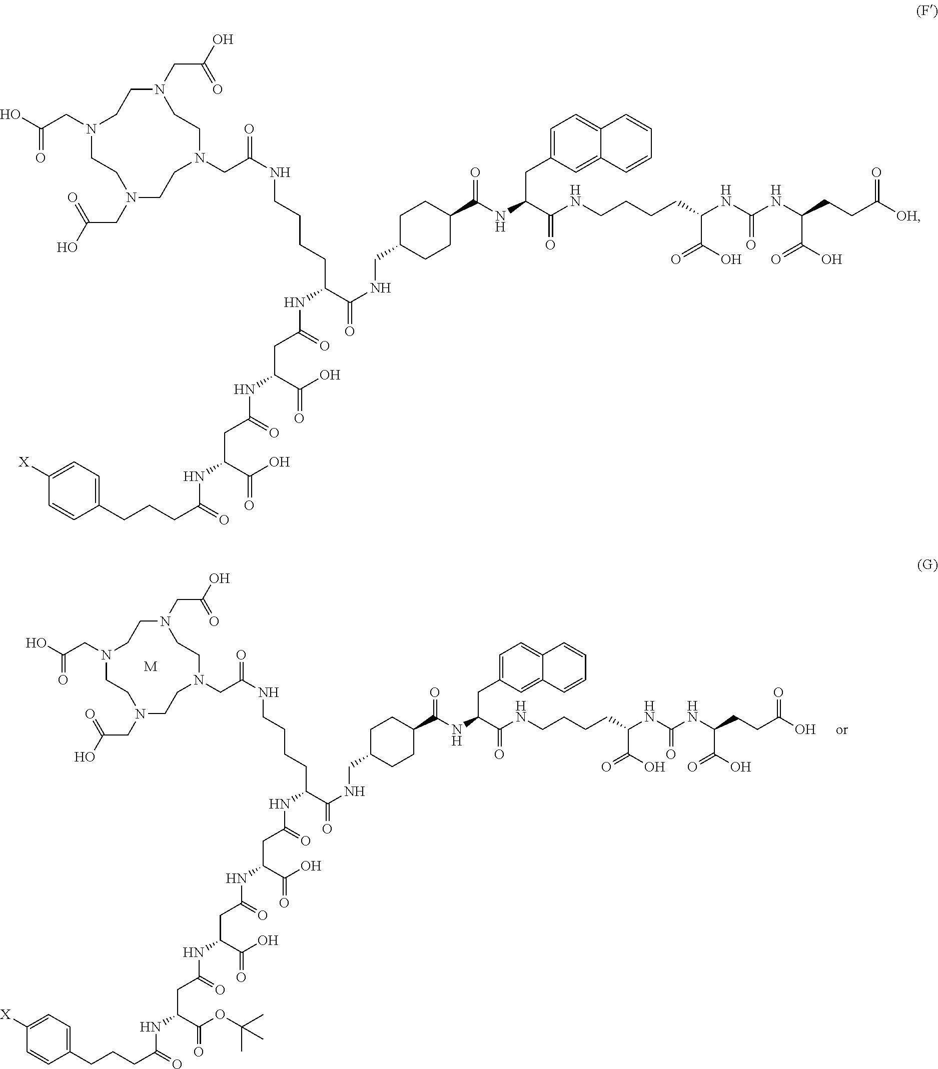

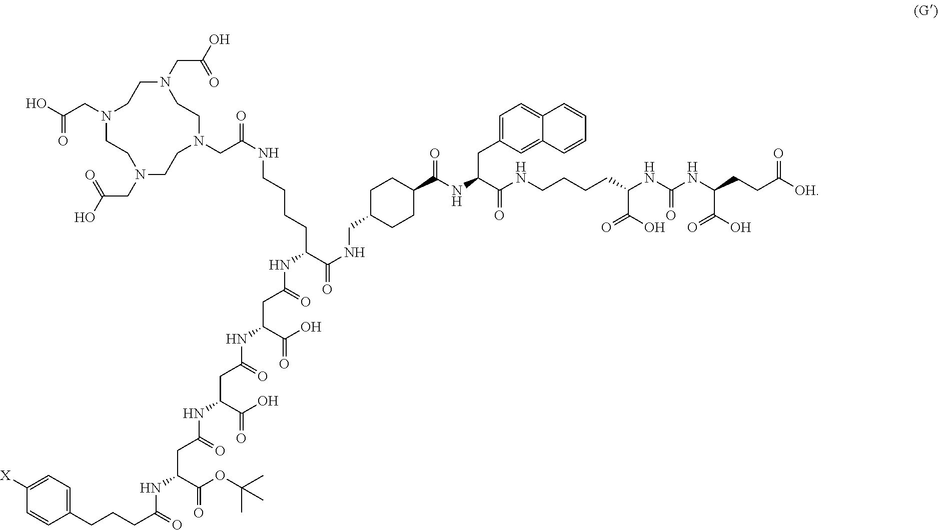

X, and M are as described above.

Exemplary radiopharmaceutical agent comprises one of the following structures (referred to herein as Compounds E. E′, F, F′, G and G′):

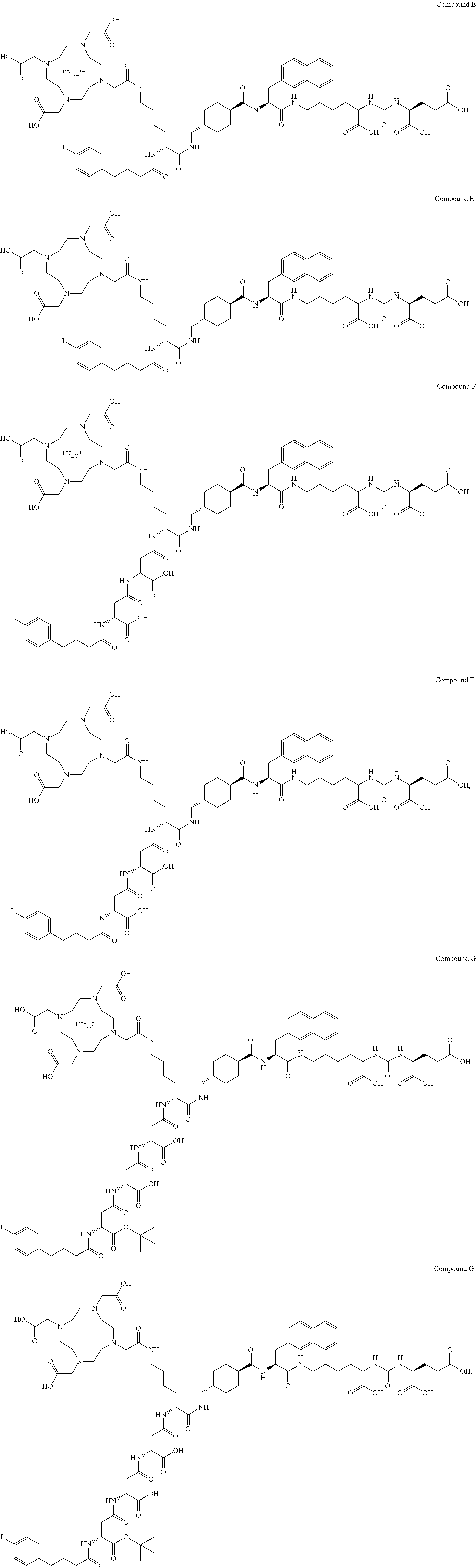

In one preferred aspect, the radiopharmaceutical agent comprises one of the following structures of Formula (H) or (H′):

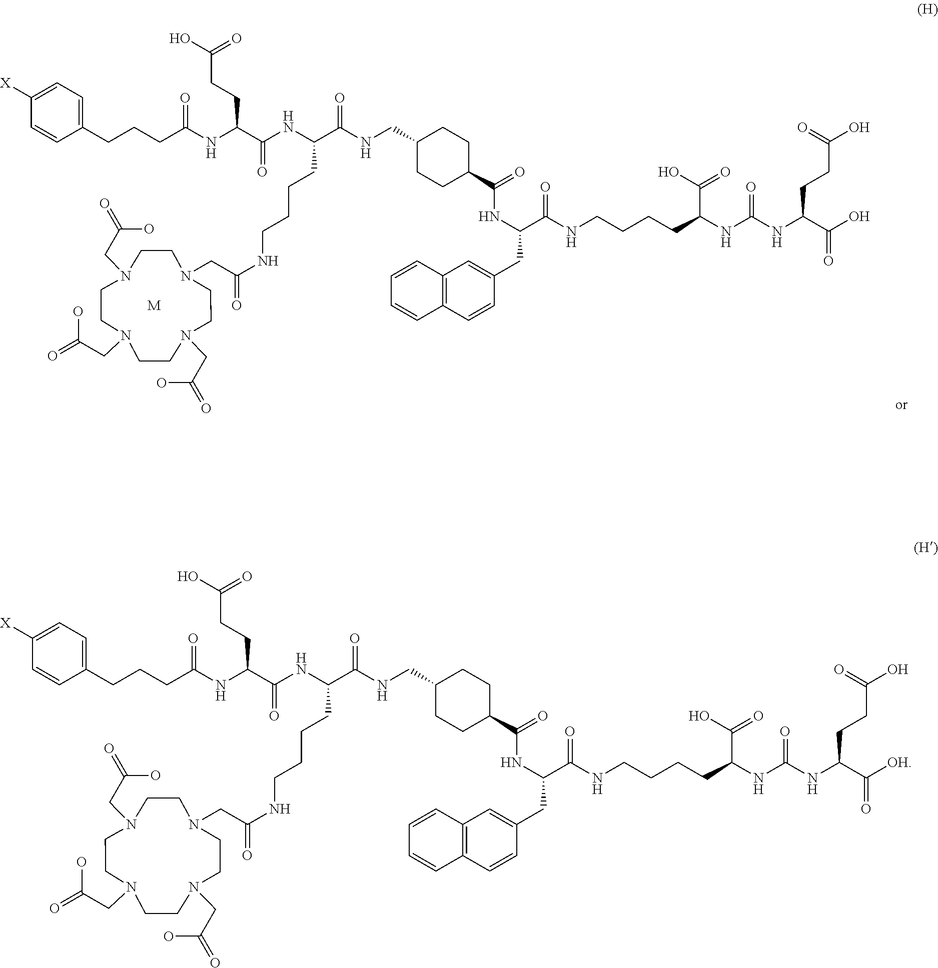

M and X are as described above.

In one preferred aspect, the radiopharmaceutical agent comprises one of the following structures (referred to herein as Compound H or H′):

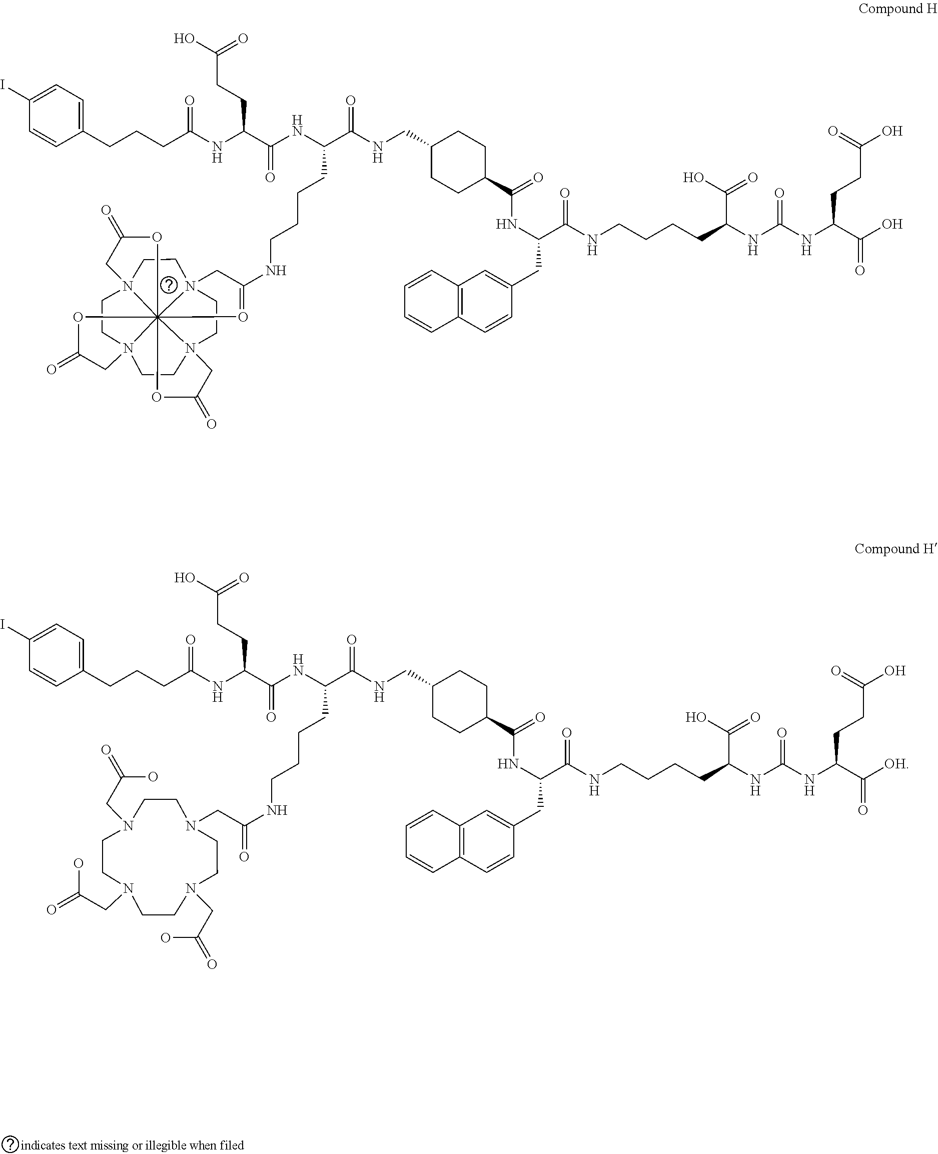

In one preferred aspect, the radiopharmaceutical agent comprises one of the following structures of Formula (J) and (J′):

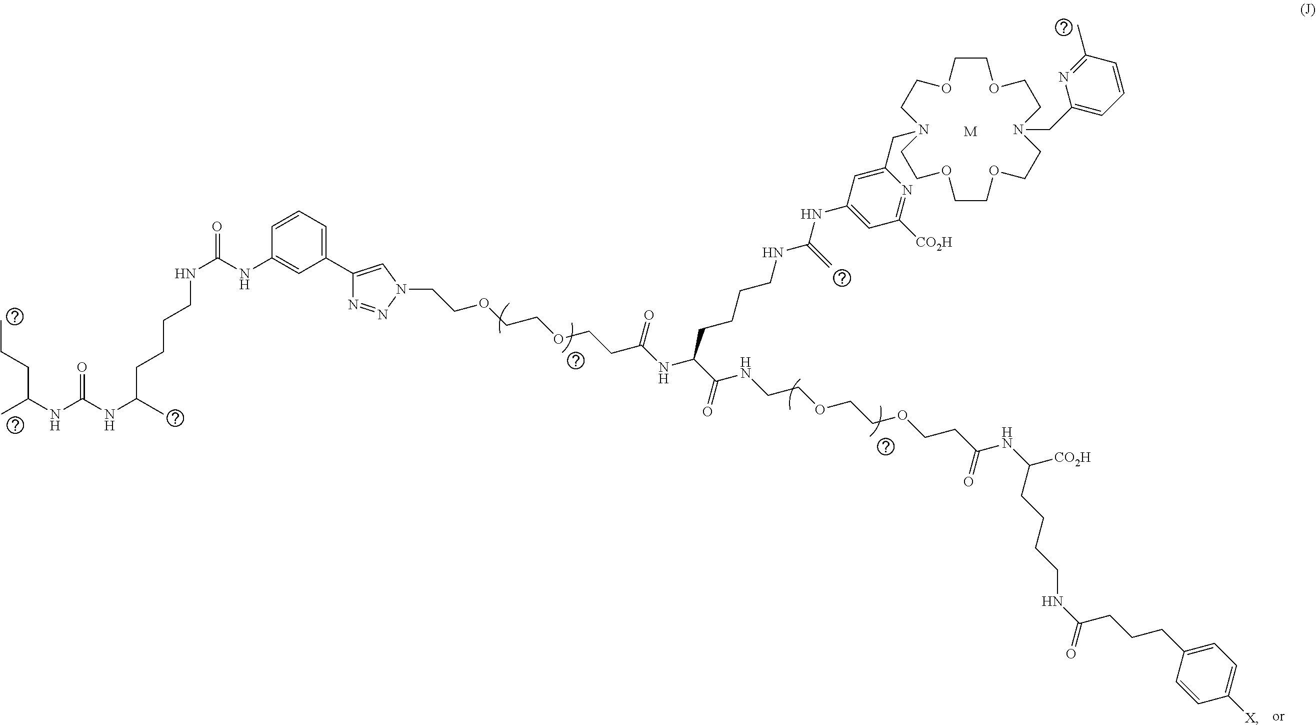

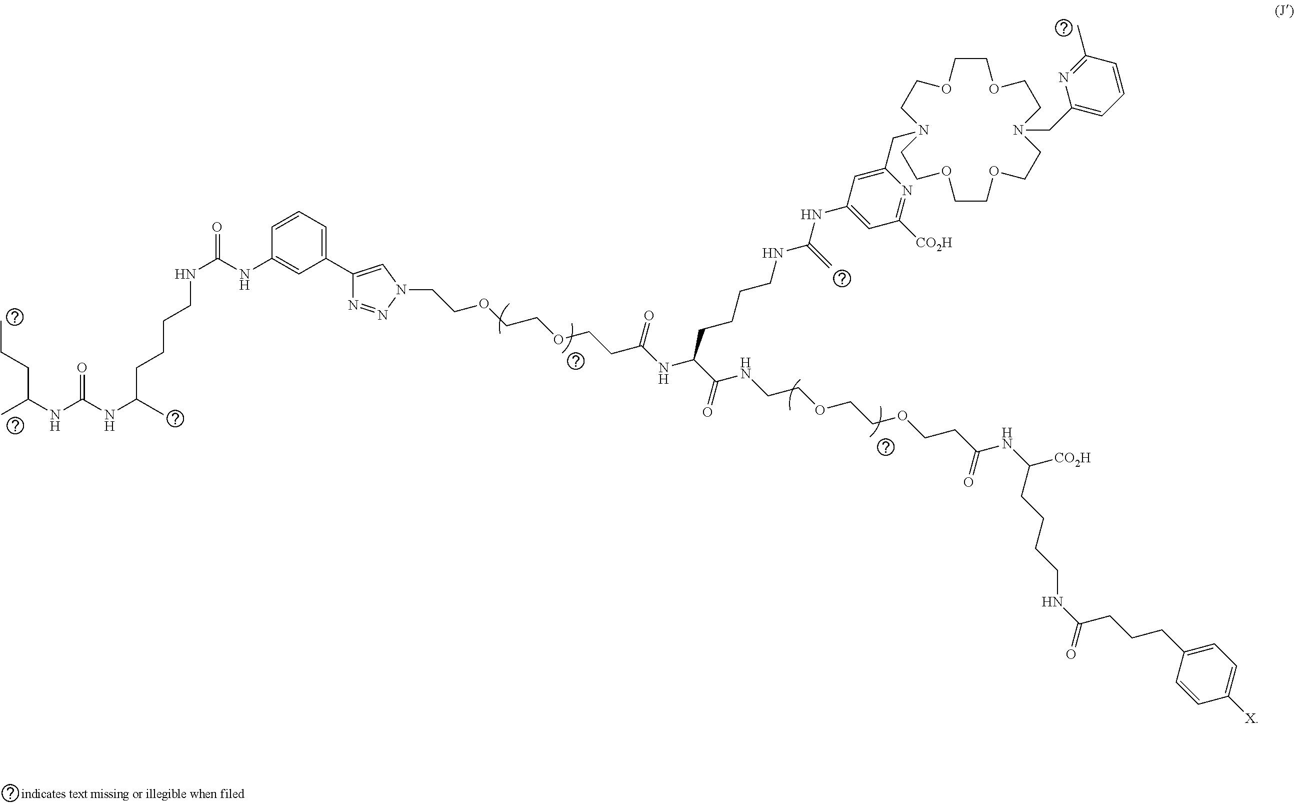

M and X are as described above.

Exemplary radiopharmaceutical agent comprises one of the following structures (referred to herein as Compound I, J, and J′):

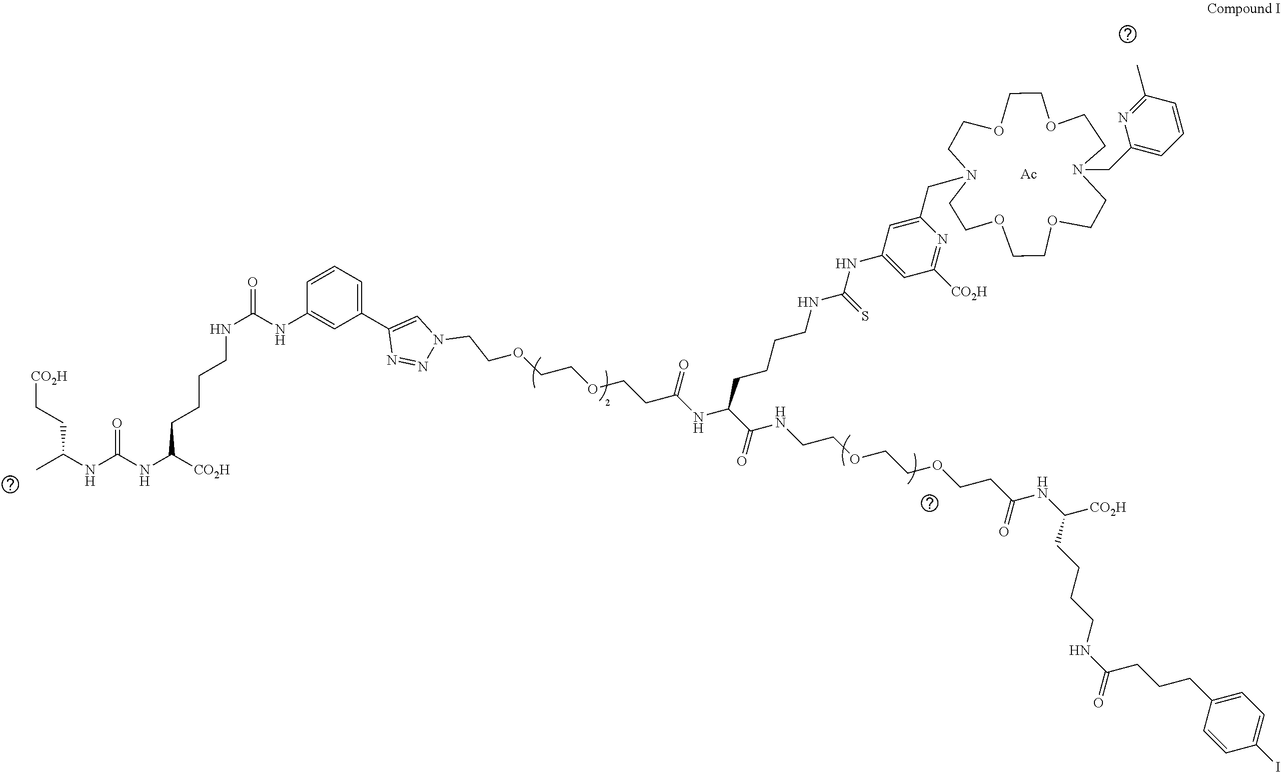

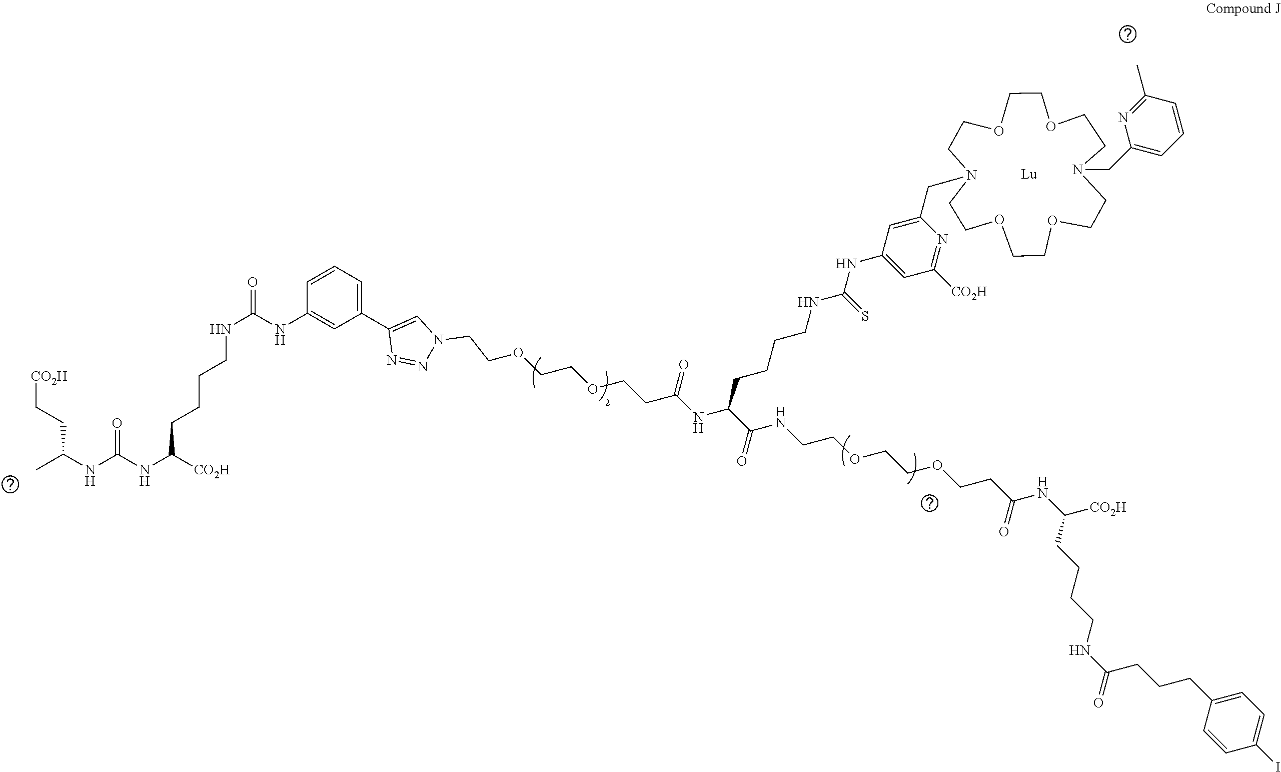

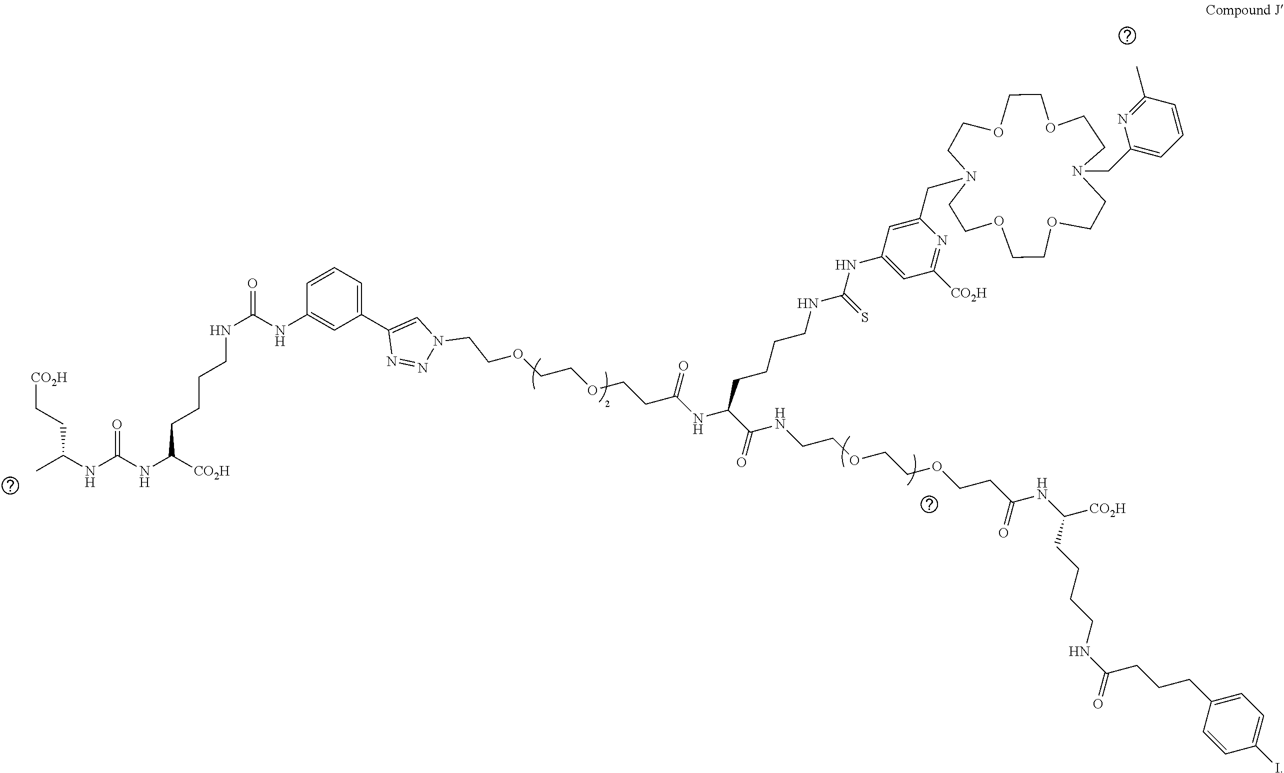

In one preferred aspect, the radiopharmaceutical agent comprises one of the following structures of Formula (K) or (K′)″

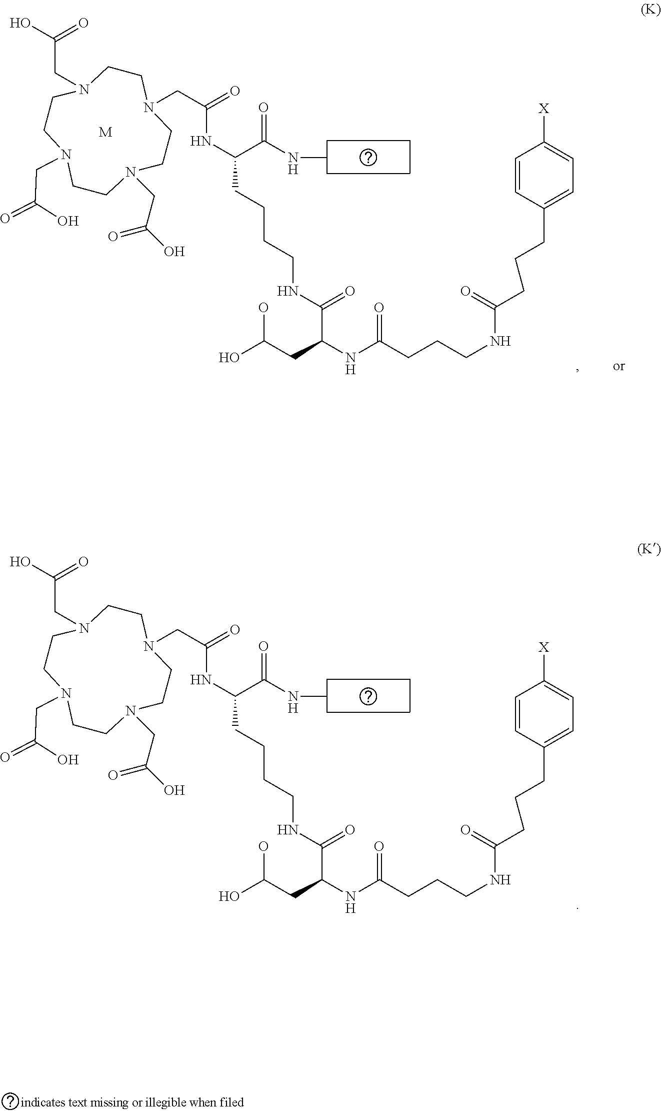

M and X are as described above.

Exemplary radiopharmaceutical agent comprises one of the following structures (referred to herein as Compound K, L, M, and M′):

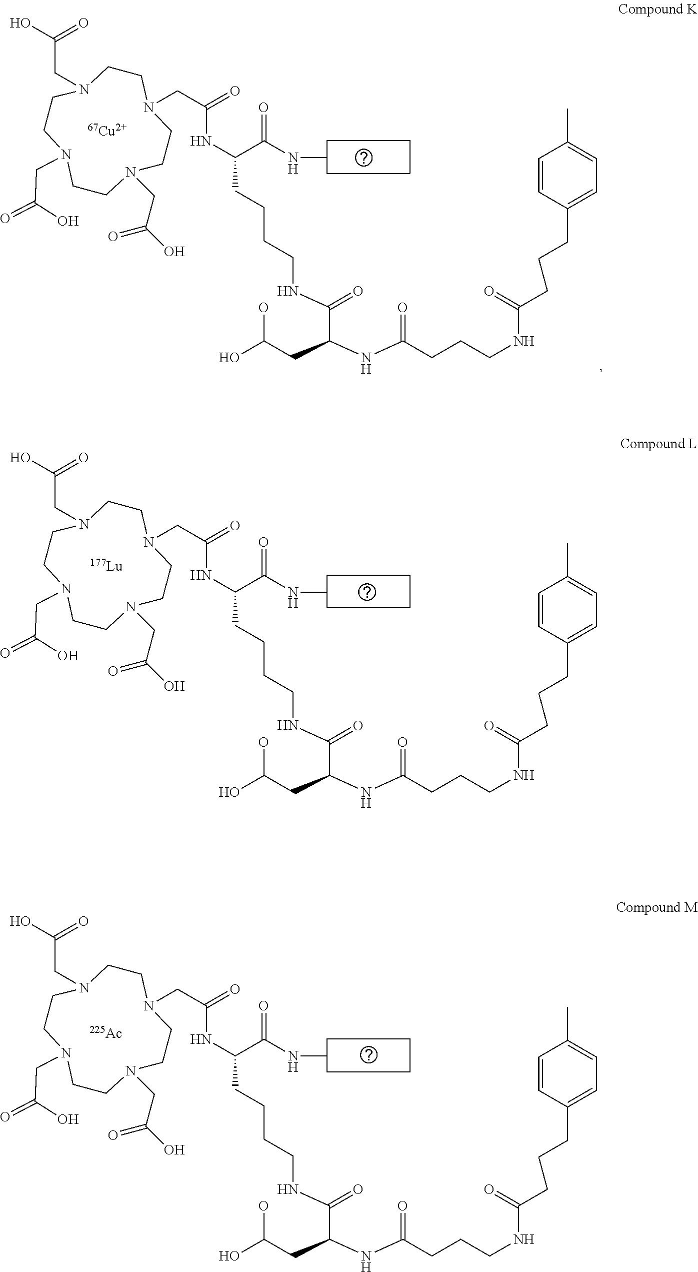

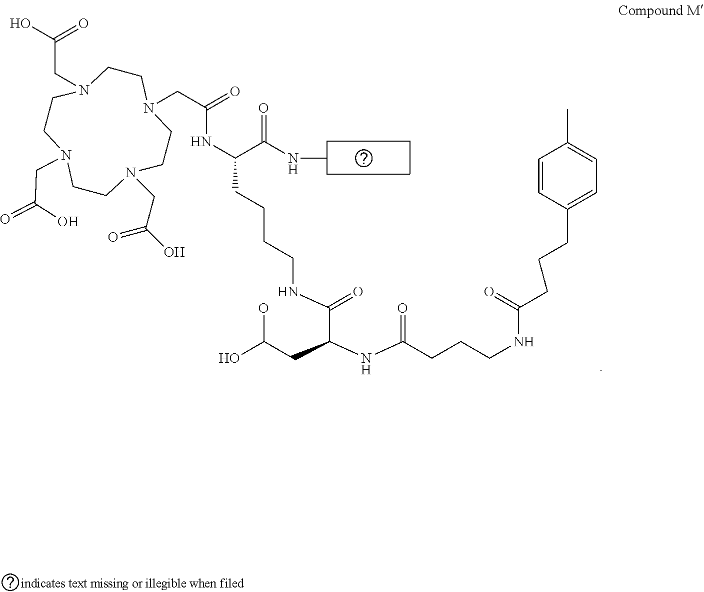 [SMILES: CNC(=O)[C@H](CCCCNC(=O)[C@H](CC(O)O)NC(=O)CCCNC(=O)CCCc1ccc(C)cc1)NC(=O)CN1CCN(CC(=O)O)CCN(CC(=O)O)CCN(CC(=O)O)CC1]

In one preferred aspect, the radiopharmaceutical agent comprises one of the following structures of Formula (N) or (N′):

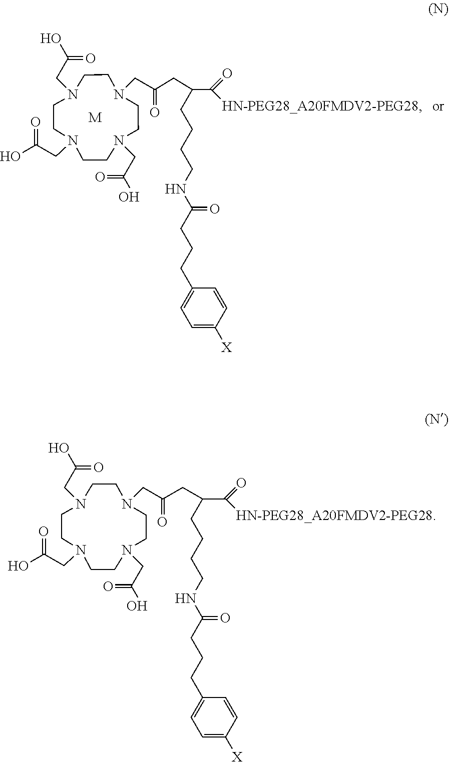

X or M are as described above.

Exemplary radiopharmaceutical agent comprises one of the following structures (referred to herein as Compound N or N′):

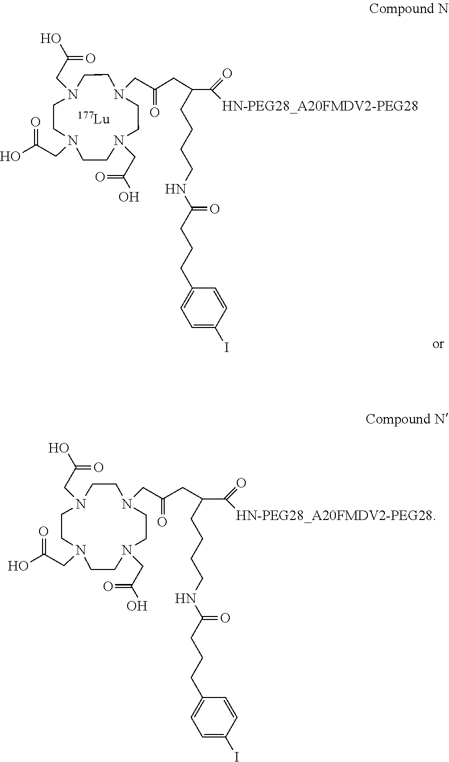

In one preferred aspect, the radiopharmaceutical agent comprises one of the following structures of Formula (O):

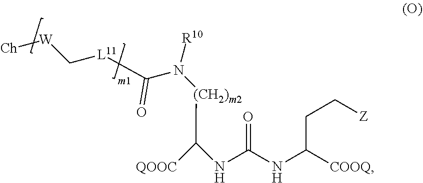

wherein:
Z is tetrazole or CO₂Q;
Q is H or a protecting group;
m2 is an integer from 1 to 5;
R¹⁰ is —CH₂—R¹¹;
R¹¹ is a substituted aryl, substituted pyridine, and unsubstituted isoquinoline, which are substituted with at least one halogen;
L¹¹ is a C₁-C₆ alkylene linker;
W is —NR² (C═O)—, —NR²—(C═S)—, —(C═O)—NR²—, and —(C═S)—NR²;
wherein each occurrence of L′ and W can be the same or different;
R¹² is H or a C₁-C₄ alkyl;
m1 is an integer of 1 to 3;
Ch is a chelating moiety that can comprise a metal (e.g., ¹⁷⁷Lu, ²²⁵Ac, ⁶⁷Cu, ²¹²Pb, ¹⁶¹Tb, or isotope ions thereof).

In some embodiments, R¹¹ is selected from the group consisting of:

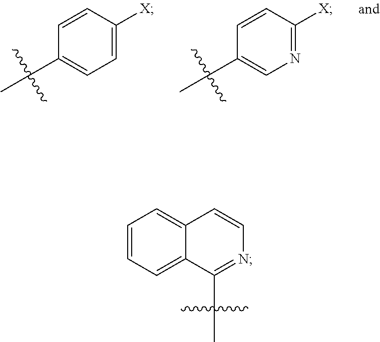

wherein X is independently Br or I.

In some embodiments, Ch is selected from the group consisting of:

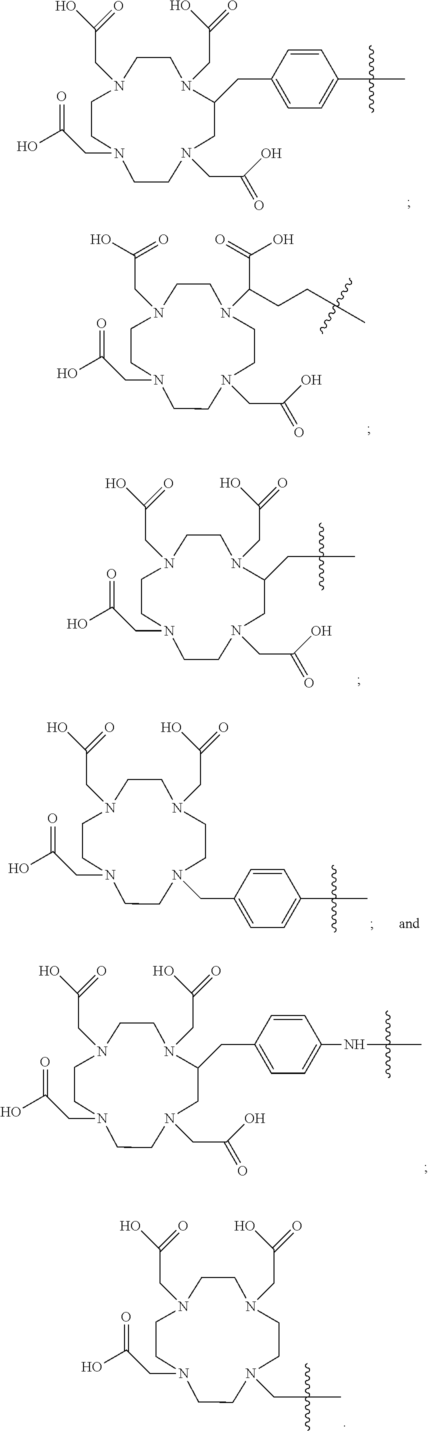

In some embodiments, the metal is a radiometal and is selected from the group consisting of: ⁶⁷Cu, ⁸⁶Y, ⁹⁰Y, ⁸⁹Zr, ¹¹¹In, ^{99m}Tc, ¹⁷⁷Lu, ¹⁵³Sm, ¹⁸⁶Re, ¹⁸⁸Re, ⁶⁷Cu, ¹⁶¹Tb, ²¹²Pb, ²²⁵Ac, ²¹³Bi, ²¹²Bi, ⁶⁷Ga, ²⁰³Pb, ⁴⁷Sc, and ¹⁶⁶Ho.

Exemplary radiopharmaceutical agents comprise one of the following structures of:

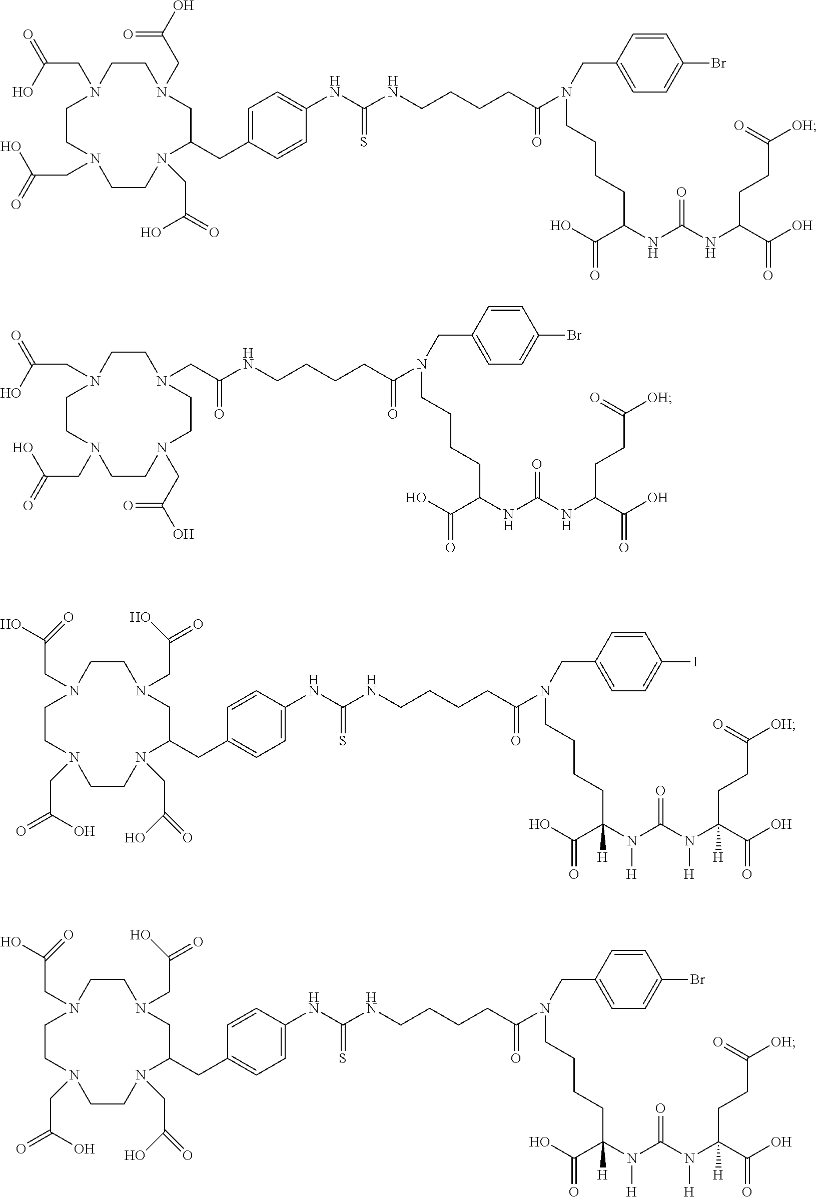

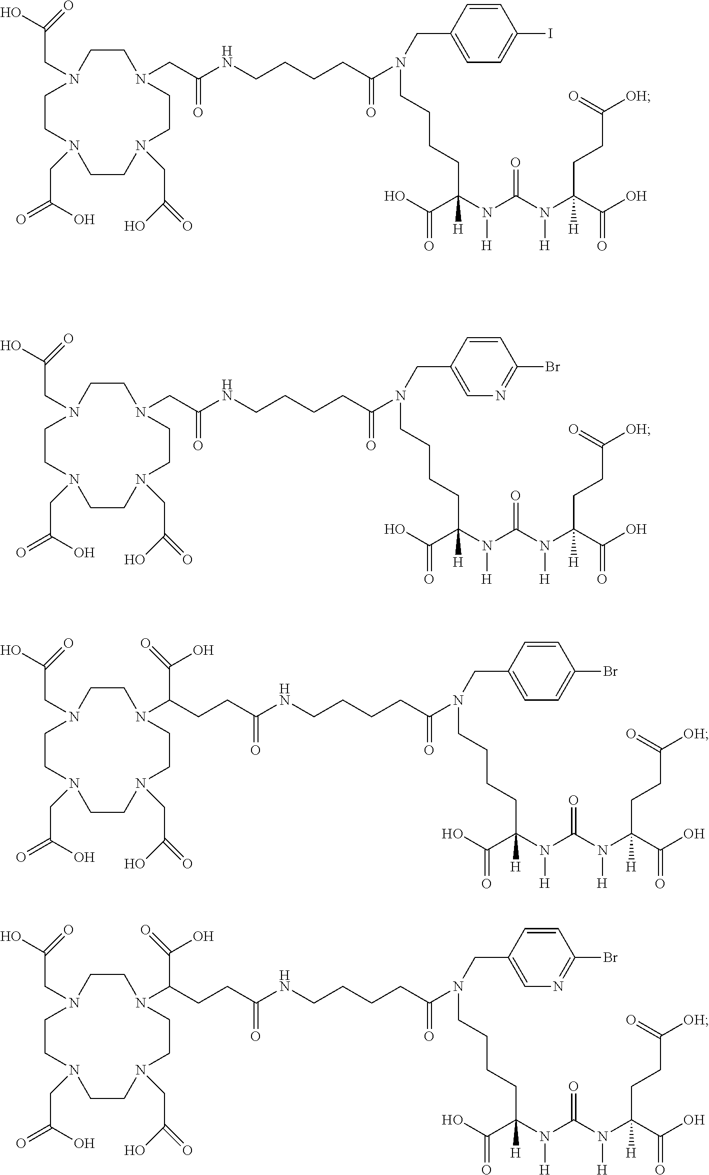

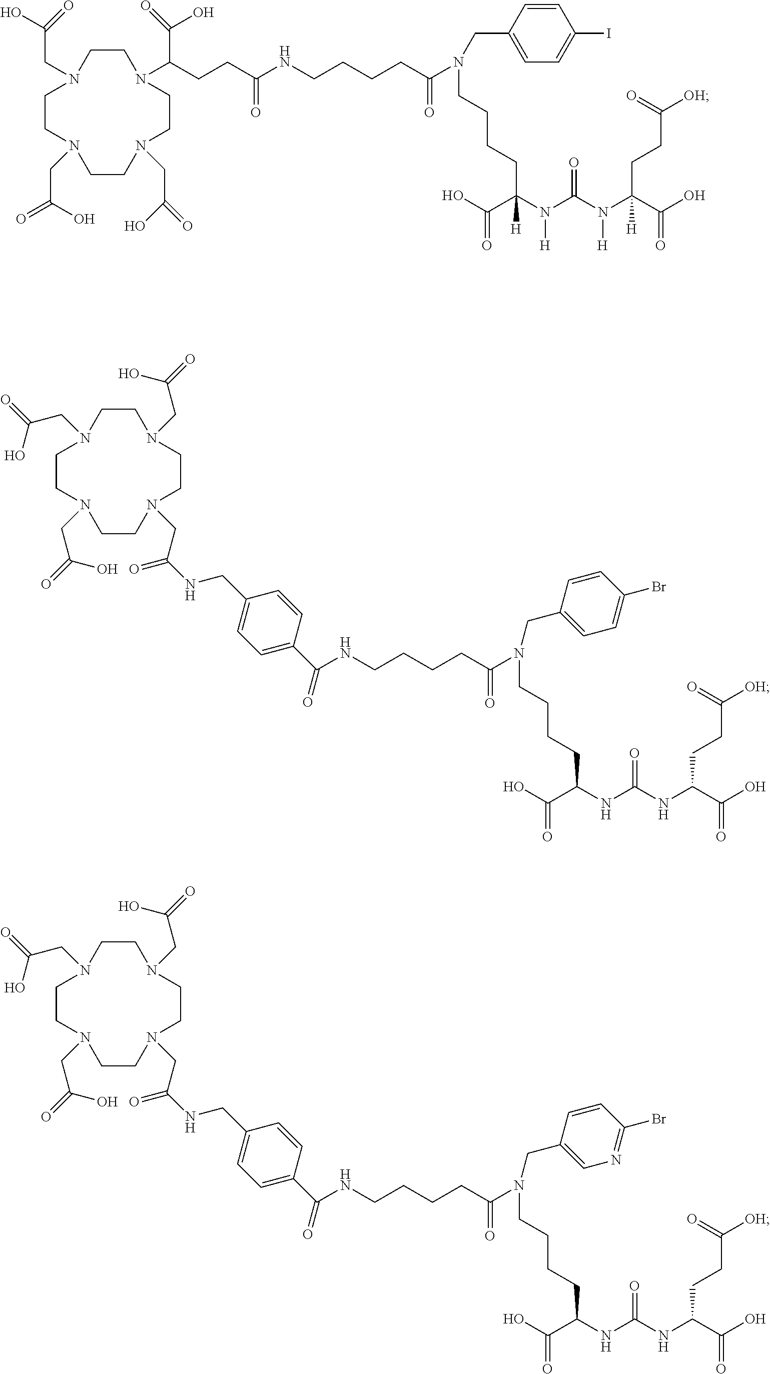 [SMILES: O=C(O)CC[C@H](NC(=O)N[C@@H](CCCCN(Cc1ccc(I)cc1)C(=O)CCCCNC(=O)CCC(C(=O)O)N1CCN(CC(=O)O)CCN(CC(=O)O)CCN(CC(=O)O)CC1)C(=O)O)C(=O)O ; O=C(O)CC[C@@H](NC(=O)N[C@H](CCCCN(Cc1ccc(Br)cc1)C(=O)CCCCNC(=O)c1ccc(CNC(=O)CN2CCN(CC(=O)O)CCN(CC(=O)O)CCN(CC(=O)O)CC2)cc1)C(=O)O)C(=O)O ; O=C(O)CC[C@@H](NC(=O)N[C@H](CCCCN(Cc1ccc(Br)nc1)C(=O)CCCCNC(=O)c1ccc(CNC(=O)CN2CCN(CC(=O)O)CCN(CC(=O)O)CCN(CC(=O)O)CC2)cc1)C(=O)O)C(=O)O]

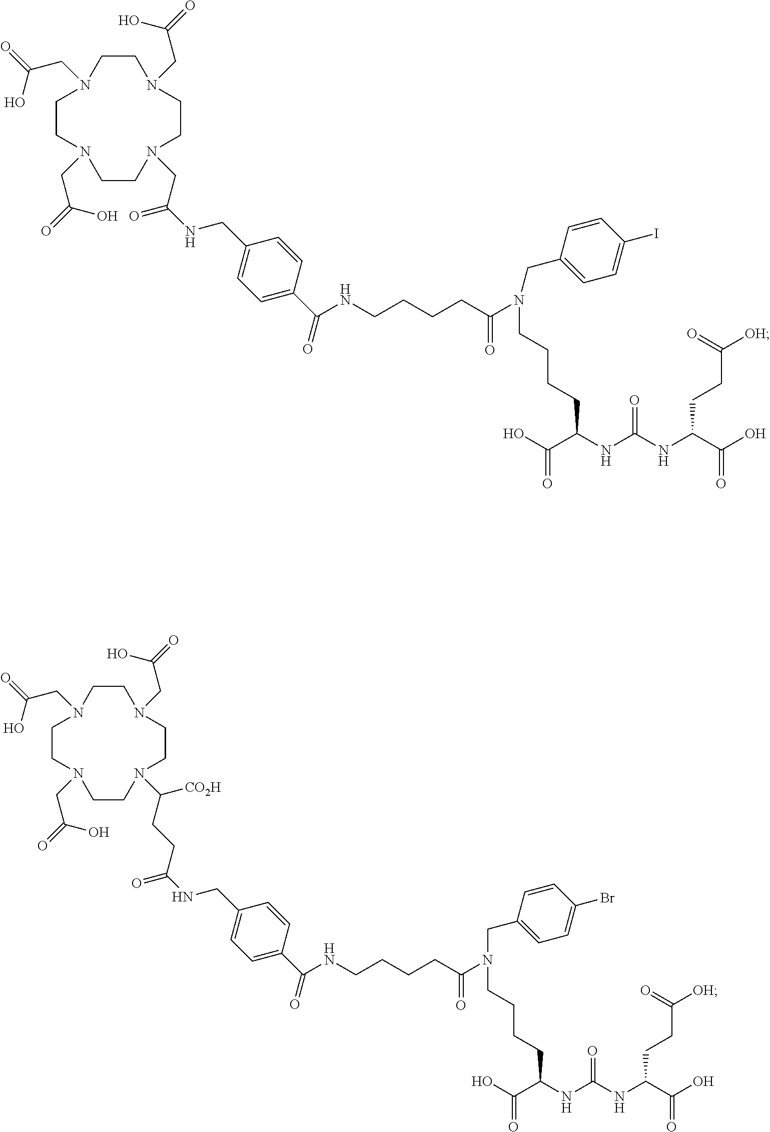 [SMILES: O=C(O)CC[C@@H](NC(=O)N[C@H](CCCCN(Cc1ccc(I)cc1)C(=O)CCCCNC(=O)c1ccc(CNC(=O)CN2CCN(CC(=O)O)CCN(CC(=O)O)CCN(CC(=O)O)CC2)cc1)C(=O)O)C(=O)O ; O=C(O)CC[C@@H](NC(=O)N[C@H](CCCCN(Cc1ccc(Br)cc1)C(=O)CCCCNC(=O)c1ccc(CNC(=O)CCC(C(=O)O)N2CCN(CC(=O)O)CCN(CC(=O)O)CCN(CC(=O)O)CC2)cc1)C(=O)O)C(=O)O]

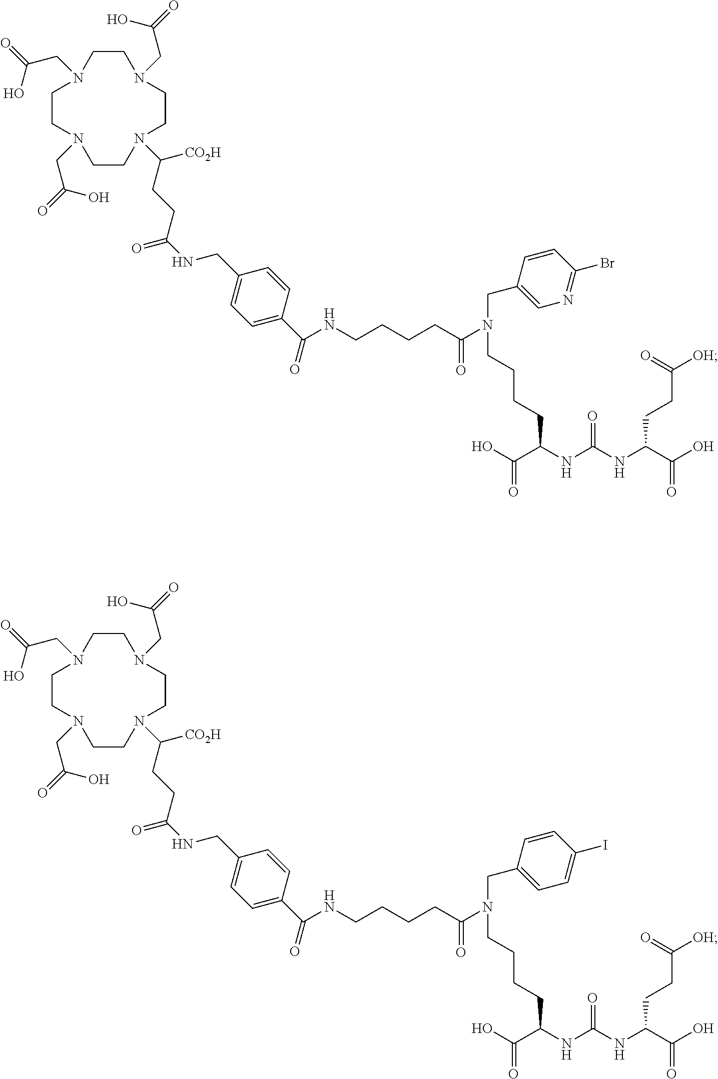 [SMILES: O=C(O)CC[C@@H](NC(=O)N[C@H](CCCCN(Cc1ccc(Br)nc1)C(=O)CCCCNC(=O)c1ccc(CNC(=O)CCC(C(=O)O)N2CCN(CC(=O)O)CCN(CC(=O)O)CCN(CC(=O)O)CC2)cc1)C(=O)O)C(=O)O ; O=C(O)CC[C@@H](NC(=O)N[C@H](CCCCN(Cc1ccc(I)cc1)C(=O)CCCCNC(=O)c1ccc(CNC(=O)CCC(C(=O)O)N2CCN(CC(=O)O)CCN(CC(=O)O)CCN(CC(=O)O)CC2)cc1)C(=O)O)C(=O)O]

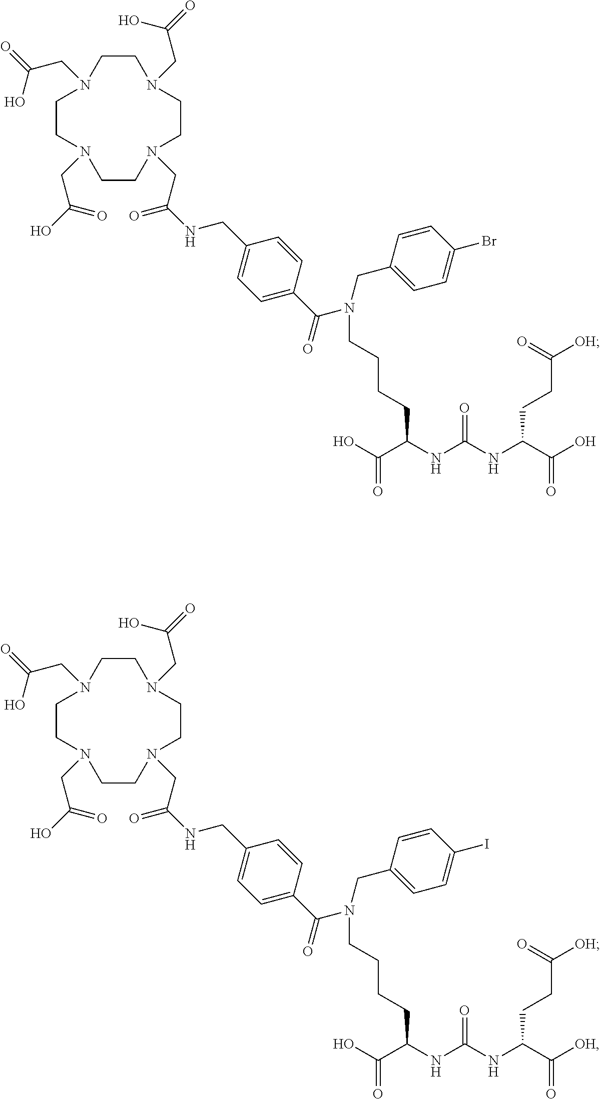 [SMILES: O=C(O)CC[C@@H](NC(=O)N[C@H](CCCCN(Cc1ccc(Br)cc1)C(=O)c1ccc(CNC(=O)CN2CCN(CC(=O)O)CCN(CC(=O)O)CCN(CC(=O)O)CC2)cc1)C(=O)O)C(=O)O ; O=C(O)CC[C@@H](NC(=O)N[C@H](CCCCN(Cc1ccc(I)cc1)C(=O)c1ccc(CNC(=O)CN2CCN(CC(=O)O)CCN(CC(=O)O)CCN(CC(=O)O)CC2)cc1)C(=O)O)C(=O)O]

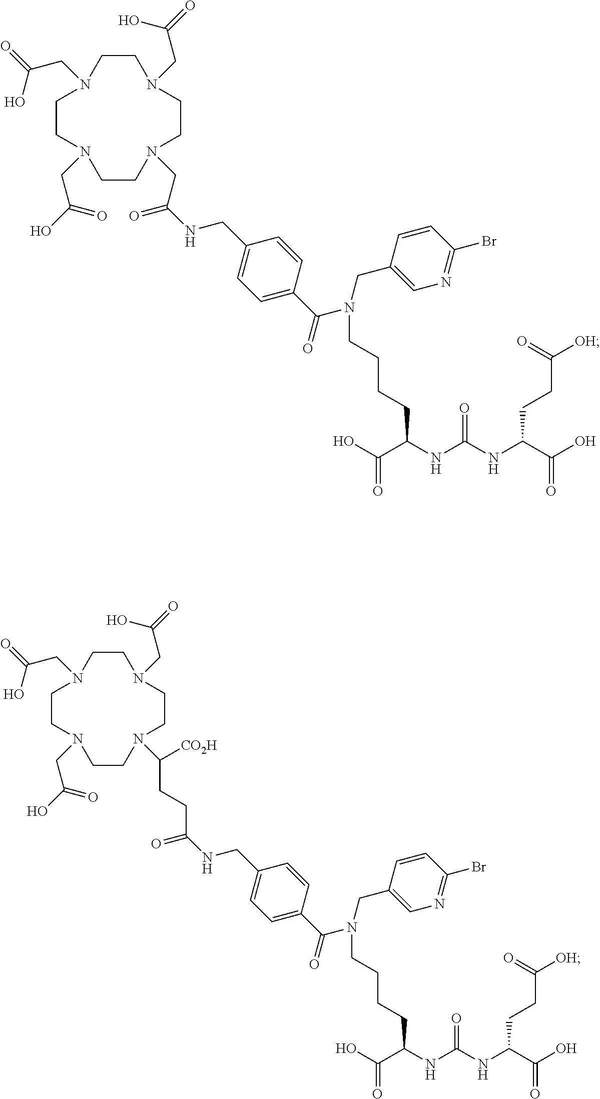 [SMILES: O=C(O)CC[C@@H](NC(=O)N[C@H](CCCCN(Cc1ccc(Br)nc1)C(=O)c1ccc(CNC(=O)CN2CCN(CC(=O)O)CCN(CC(=O)O)CCN(CC(=O)O)CC2)cc1)C(=O)O)C(=O)O ; O=C(O)CC[C@@H](NC(=O)N[C@H](CCCCN(Cc1ccc(Br)nc1)C(=O)c1ccc(CNC(=O)CCC(C(=O)O)N2CCN(CC(=O)O)CCN(CC(=O)O)CCN(CC(=O)O)CC2)cc1)C(=O)O)C(=O)O]

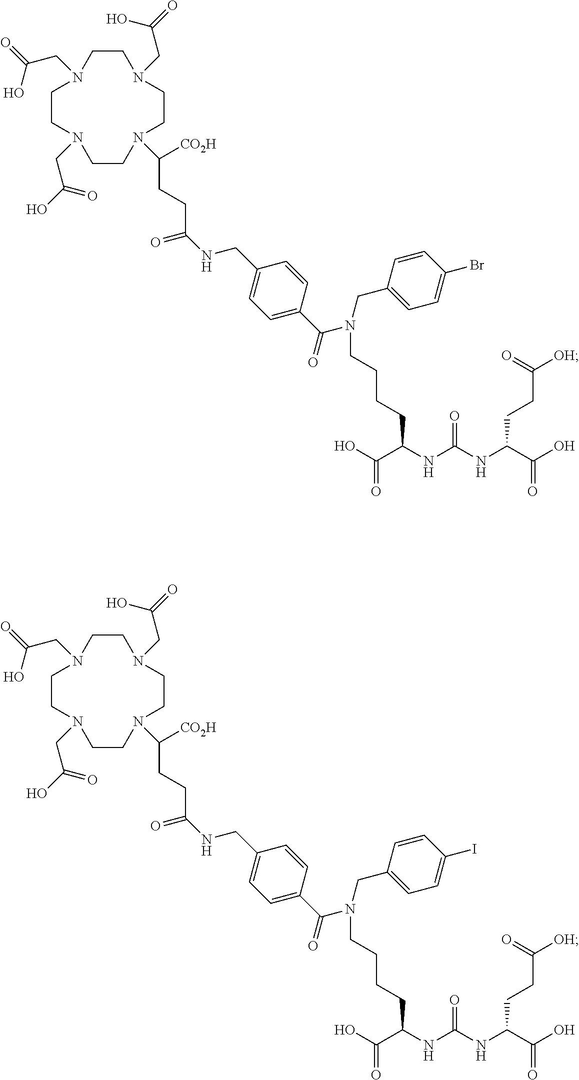 [SMILES: O=C(O)CC[C@@H](NC(=O)N[C@H](CCCCN(Cc1ccc(Br)cc1)C(=O)c1ccc(CNC(=O)CCC(C(=O)O)N2CCN(CC(=O)O)CCN(CC(=O)O)CCN(CC(=O)O)CC2)cc1)C(=O)O)C(=O)O ; O=C(O)CC[C@@H](NC(=O)N[C@H](CCCCN(Cc1ccc(I)cc1)C(=O)c1ccc(CNC(=O)CCC(C(=O)O)N2CCN(CC(=O)O)CCN(CC(=O)O)CCN(CC(=O)O)CC2)cc1)C(=O)O)C(=O)O]

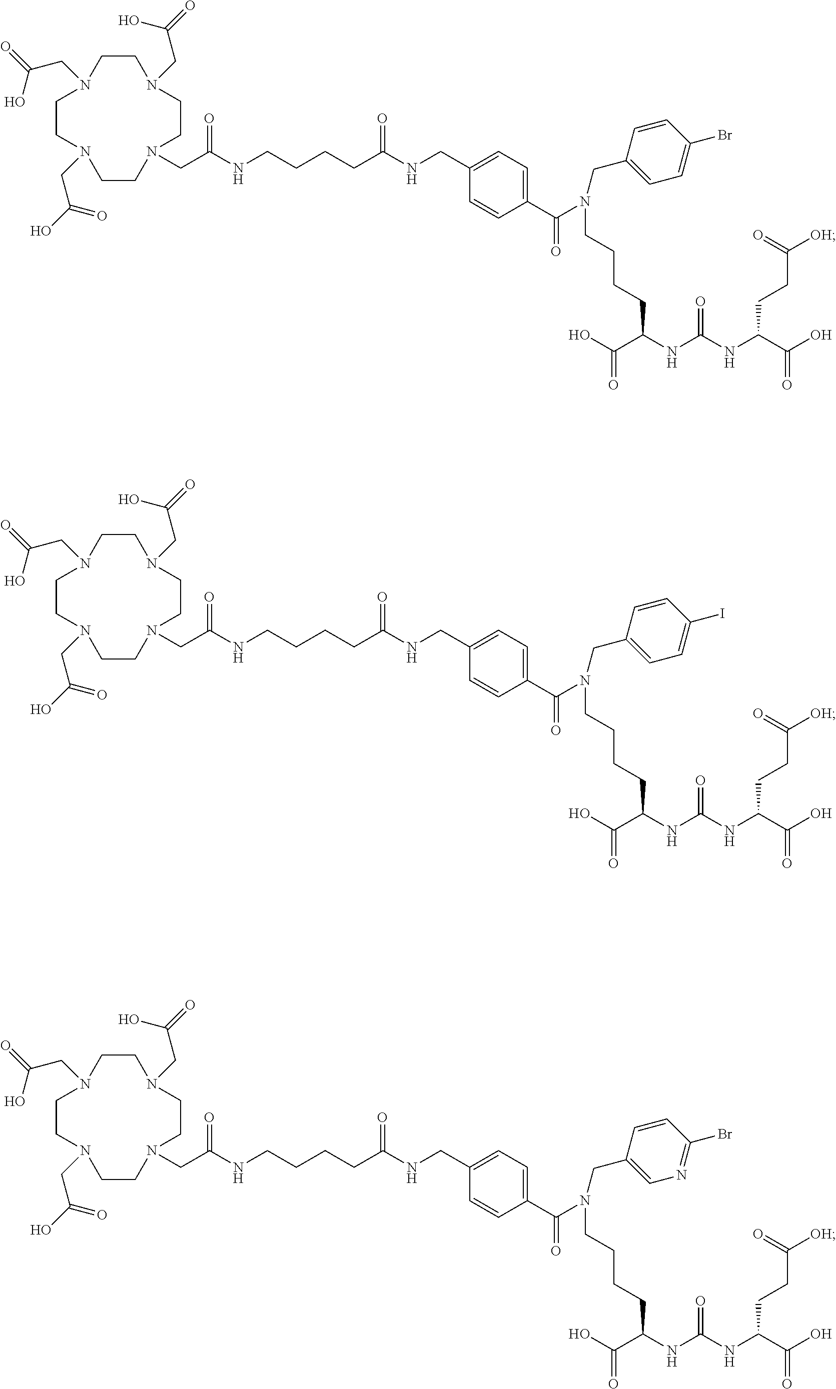 [SMILES: O=C(O)CC[C@@H](NC(=O)N[C@H](CCCCN(Cc1ccc(Br)cc1)C(=O)c1ccc(CNC(=O)CCCCNC(=O)CN2CCN(CC(=O)O)CCN(CC(=O)O)CCN(CC(=O)O)CC2)cc1)C(=O)O)C(=O)O ; O=C(O)CC[C@@H](NC(=O)N[C@H](CCCCN(Cc1ccc(I)cc1)C(=O)c1ccc(CNC(=O)CCCCNC(=O)CN2CCN(CC(=O)O)CCN(CC(=O)O)CCN(CC(=O)O)CC2)cc1)C(=O)O)C(=O)O ; O=C(O)CC[C@@H](NC(=O)N[C@H](CCCCN(Cc1ccc(Br)nc1)C(=O)c1ccc(CNC(=O)CCCCNC(=O)CN2CCN(CC(=O)O)CCN(CC(=O)O)CCN(CC(=O)O)CC2)cc1)C(=O)O)C(=O)O]

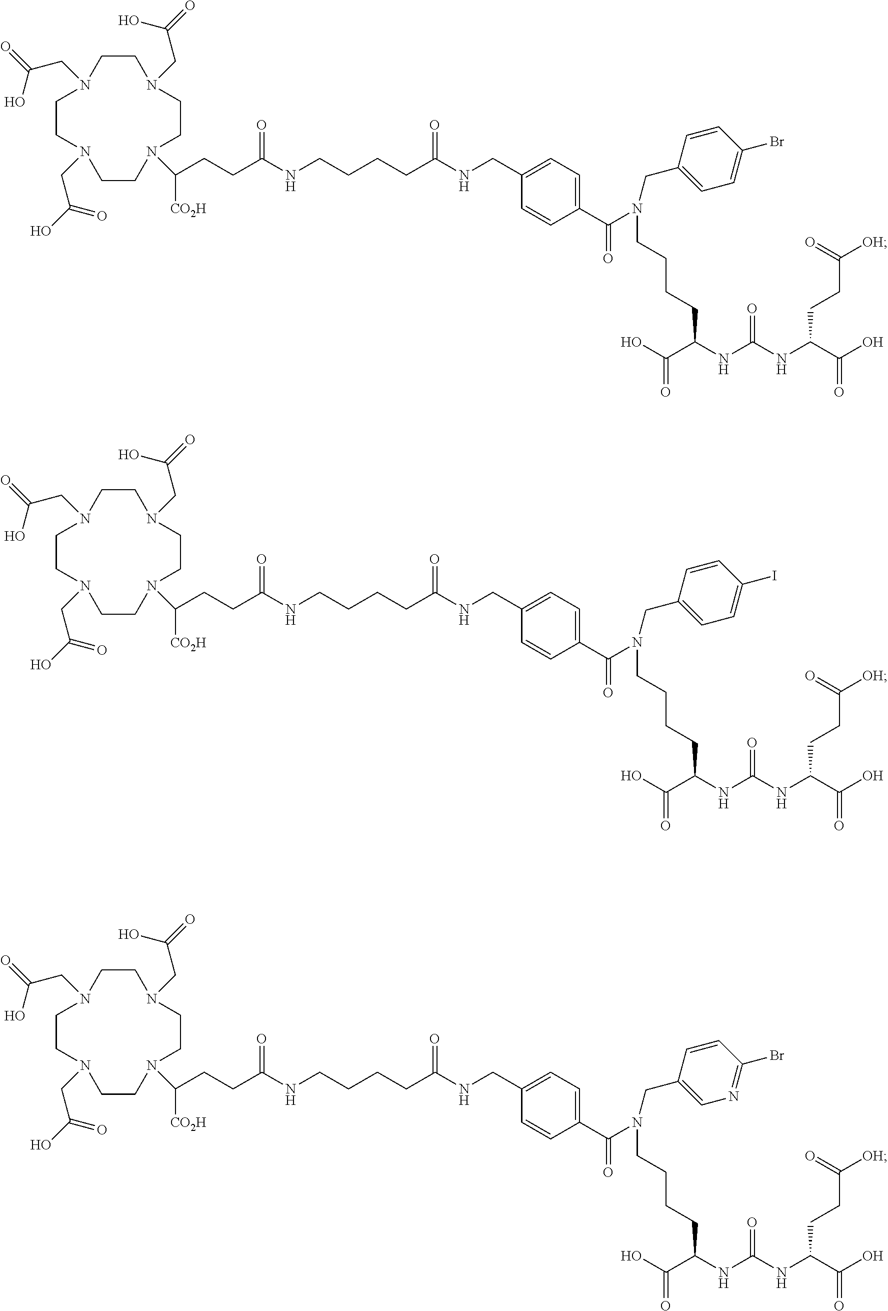 [SMILES: O=C(O)CC[C@@H](NC(=O)N[C@H](CCCCN(Cc1ccc(Br)cc1)C(=O)c1ccc(CNC(=O)CCCCNC(=O)CCC(C(=O)O)N2CCN(CC(=O)O)CCN(CC(=O)O)CCN(CC(=O)O)CC2)cc1)C(=O)O)C(=O)O ; O=C(O)CC[C@@H](NC(=O)N[C@H](CCCCN(Cc1ccc(I)cc1)C(=O)c1ccc(CNC(=O)CCCCNC(=O)CCC(C(=O)O)N2CCN(CC(=O)O)CCN(CC(=O)O)CCN(CC(=O)O)CC2)cc1)C(=O)O)C(=O)O ; O=C(O)CC[C@@H](NC(=O)N[C@H](CCCCN(Cc1ccc(Br)nc1)C(=O)c1ccc(CNC(=O)CCCCNC(=O)CCC(C(=O)O)N2CCN(CC(=O)O)CCN(CC(=O)O)CCN(CC(=O)O)CC2)cc1)C(=O)O)C(=O)O]

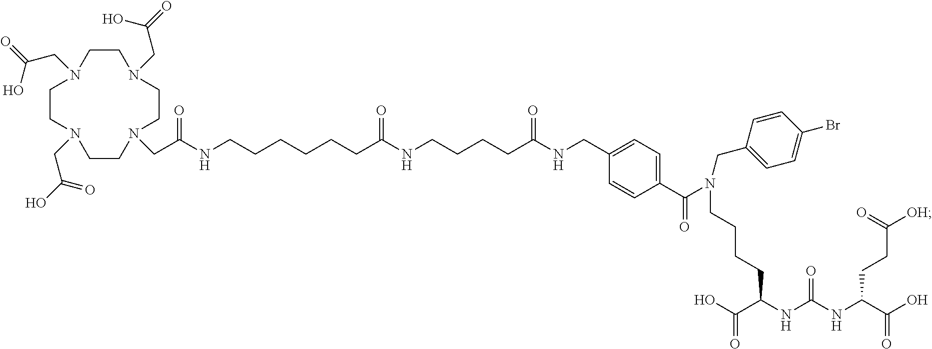 [SMILES: O=C(O)CC[C@@H](NC(=O)N[C@H](CCCCN(Cc1ccc(Br)cc1)C(=O)c1ccc(CNC(=O)CCCCNC(=O)CCCCCCNC(=O)CN2CCN(CC(=O)O)CCN(CC(=O)O)CCN(CC(=O)O)CC2)cc1)C(=O)O)C(=O)O]

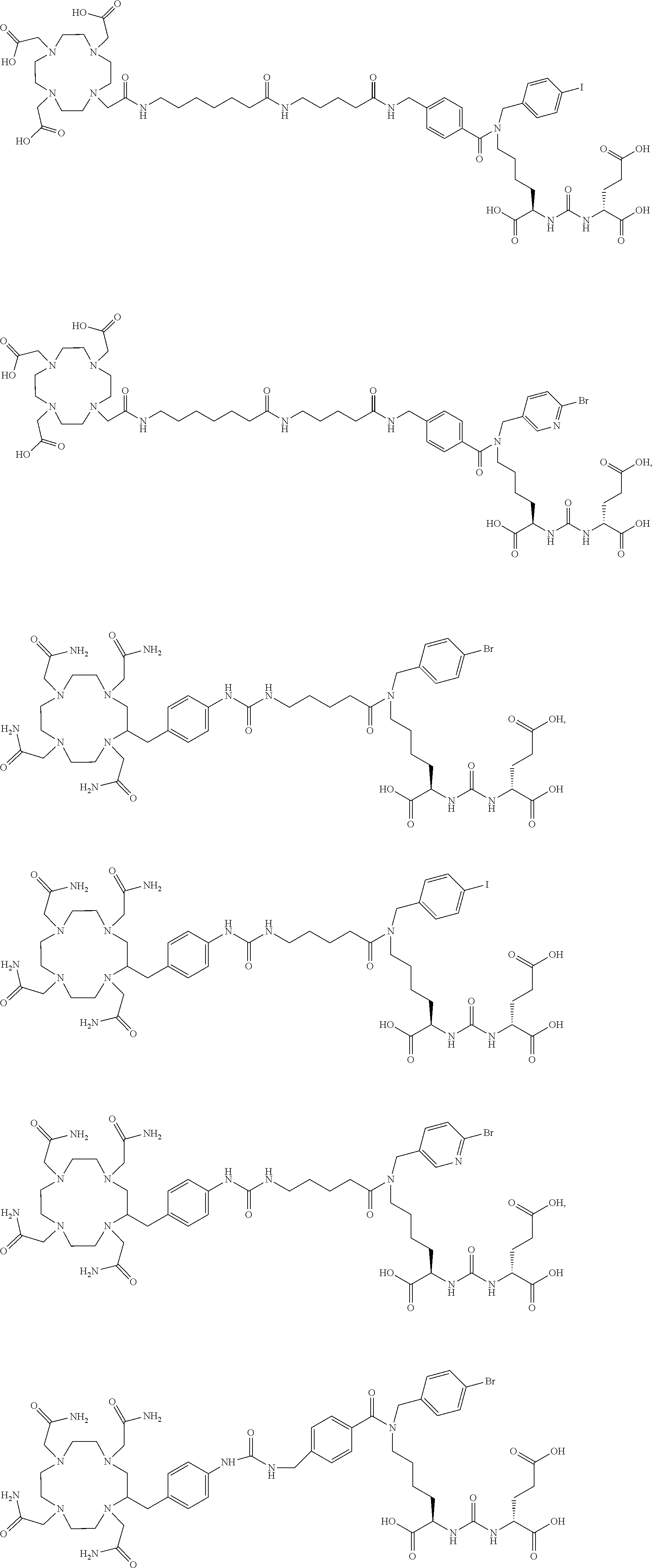

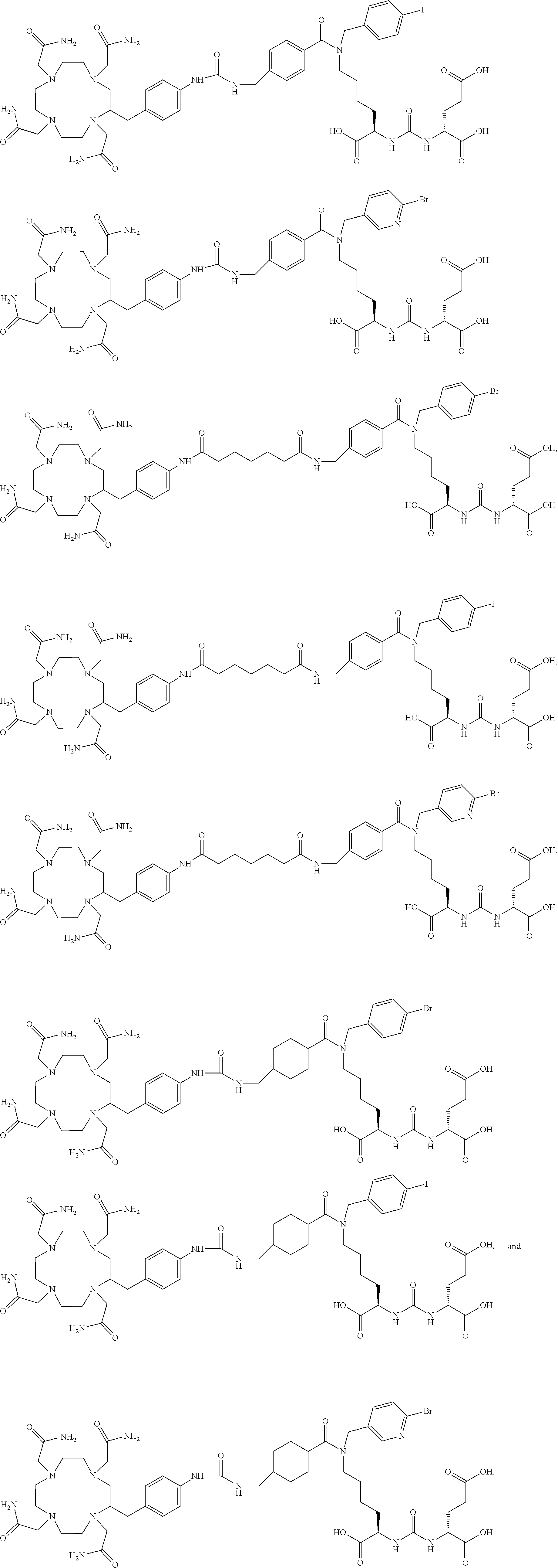

Additional exemplary radiopharmaceutical agents comprise one of the following structures of:

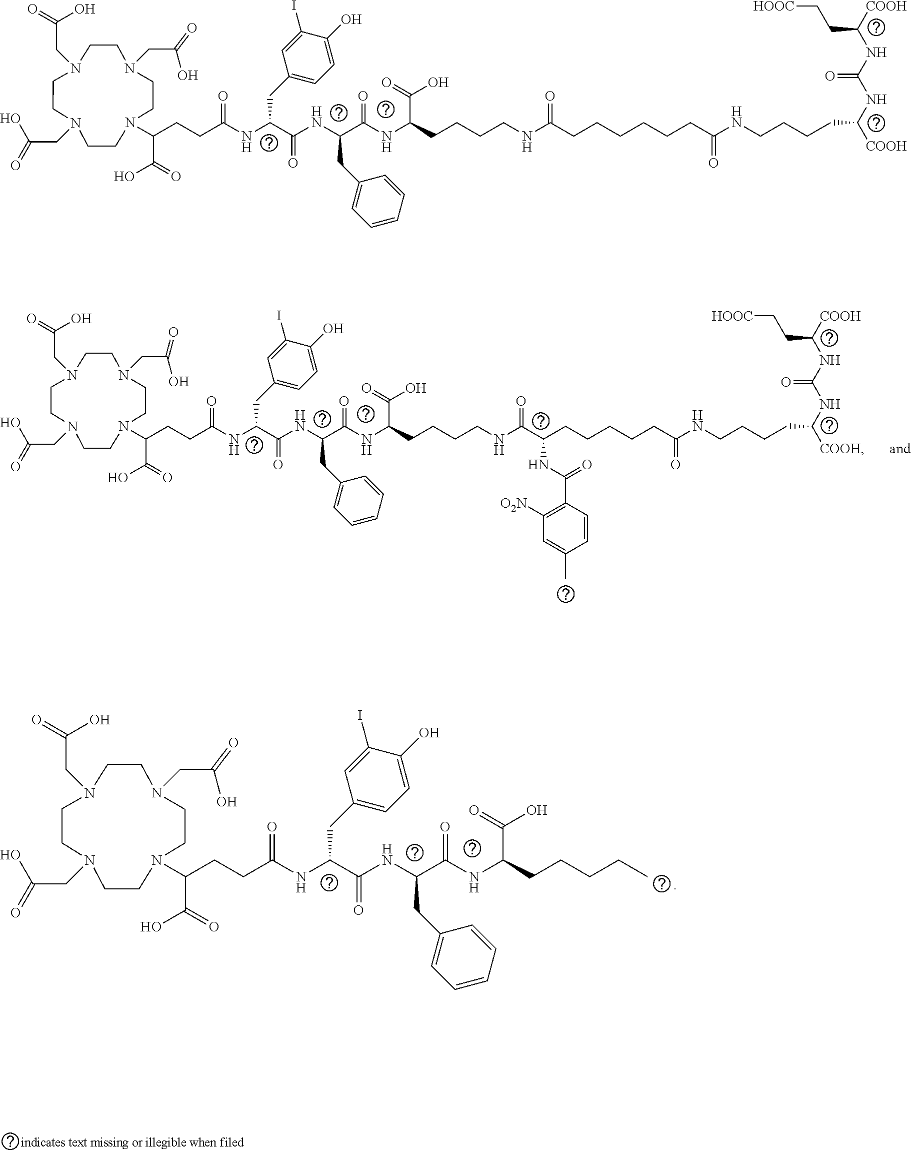

More exemplary radiopharmaceutical agents comprise one of the following structures of:

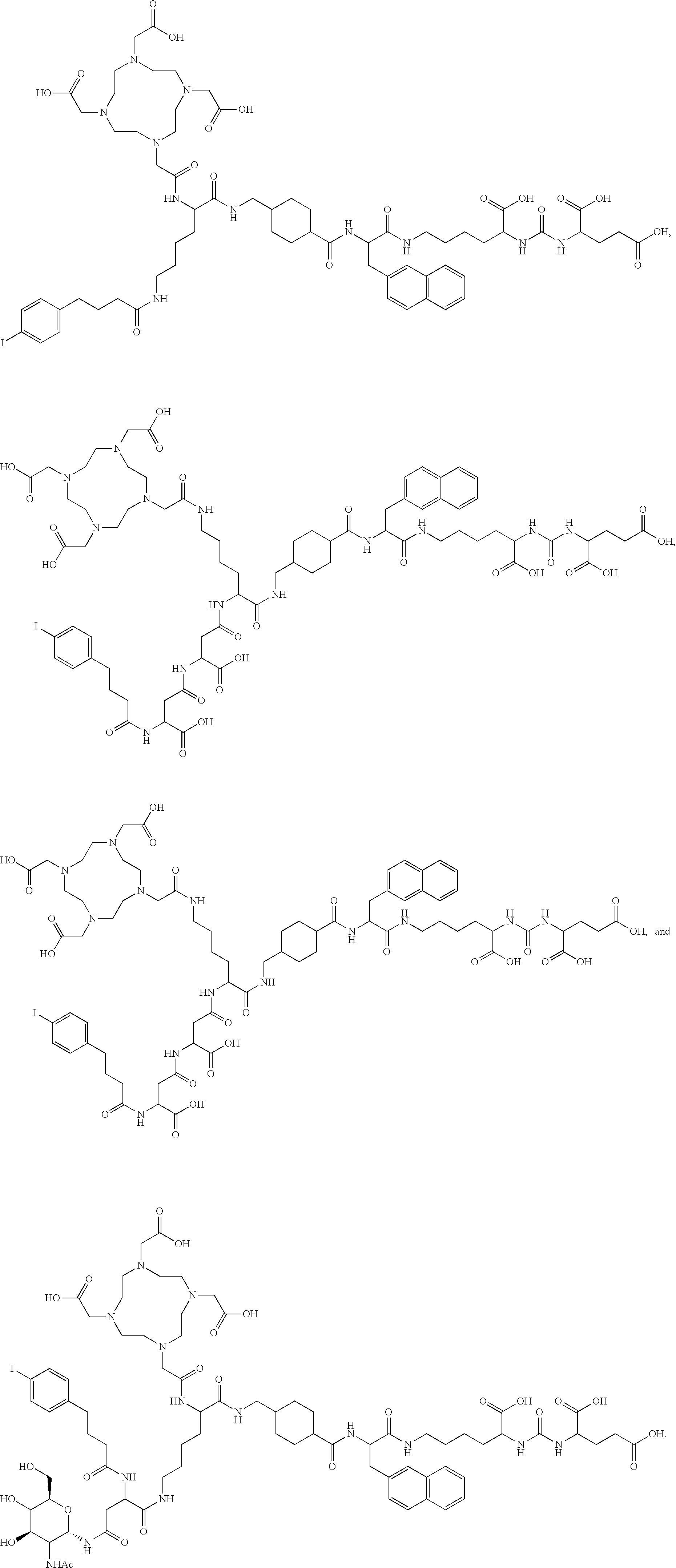

Contents and compounds (e.g., radiopharmaceutical agents) of Brandt et al, Nuclear Medicine and Biology, 70 (2019) 46-52; Yao et al., Bioorganic & Medicinal Chemistry 28 (2020) 115319; Kelly et al., Journal of Nuclear Medicine vol. 58, no. 9 1442-Bioorganic & Medicinal Chemistry, 2017, 58 (9): 1442-1449; Benesova et al., Mol. Pharmaceutics, (2018) 15, 3, 934-946; Kuo et al., Mol. Pharmaceutics, (2018) 15, 11, 5183-5191; Kelly et al., J. Nucl Med 2019, 60:649-655; Davis, Pharmaceutics 2022, 14, 74; and Huynh et al., 2022 Pharmaceuticals, 15, 299; WO2017/165473; WO2018/215627; and WO2018/215627 are incorporated into the present disclosure in their entirety.

In one preferred aspect of the specification, the radiopharmaceutical agent comprises one of the compounds in the following Table 5.

TABLE 5

| Compounds | Structure |
|---|---|
| TCMC- PSMA I&T MW:1423.37 Formula: C₆₀H₉₁IN₁₄O₁₈ | 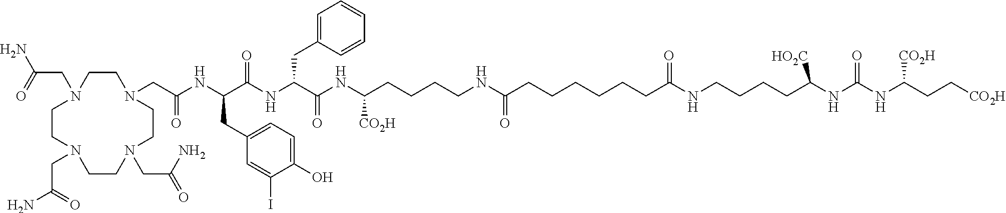 [SMILES: NC(=O)CN1CCN(CC(N)=O)CCN(CC(=O)N[C@H](Cc2ccc(O)c(I)c2)C(=O)N[C@H](Cc2ccccc2)C(=O)N[C@H](CCCCNC(=O)CCCCCCC(=O)NCCCC[C@H](NC(=O)N[C@@H](CCC(=O)O)C(=O)O)C(=O)O)C(=O)O)CCN(CC(N)=O)CC1] |
| p-SCN-Bn- TCMC- PSMA I&T MW: 1587.60 Formula: C₆₈H₉₉IN₁₆O₁₈S | 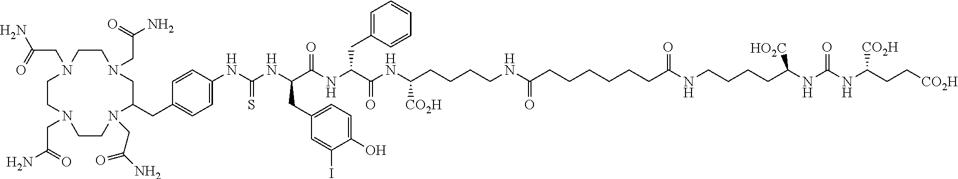 [SMILES: NC(=O)CN1CCN(CC(N)=O)CCN(CC(N)=O)C(Cc2ccc(NC(=S)N[C@H](Cc3ccc(O)c(I)c3)C(=O)N[C@H](Cc3ccccc3)C(=O)N[C@H](CCCCNC(=O)CCCCCCC(=O)NCCCC[C@H](NC(=O)N[C@@H](CCC(=O)O)C(=O)O)C(=O)O)C(=O)O)cc2)CN(CC(N)=O)CC1] |
| PSC-PSMA I&T MW: 1425.34 Formula: C₆₀H₈₉IN₁₂O₂₀ | 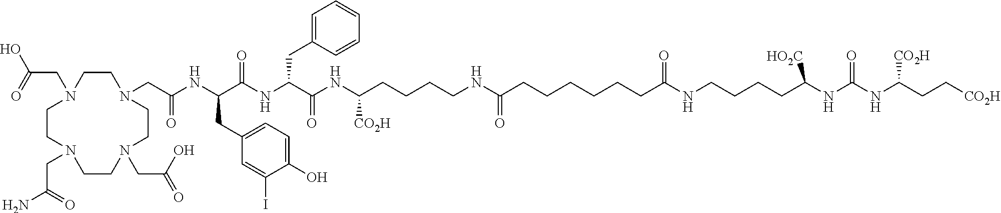 [SMILES: NC(=O)CN1CCN(CC(=O)O)CCN(CC(=O)N[C@H](Cc2ccc(O)c(I)c2)C(=O)N[C@H](Cc2ccccc2)C(=O)N[C@H](CCCCNC(=O)CCCCCCC(=O)NCCCC[C@H](NC(=O)N[C@@H](CCC(=O)O)C(=O)O)C(=O)O)C(=O)O)CCN(CC(=O)O)CC1] |

Compounds in Table 5 may be suitably synthesized using solid phase synthesis. For example, protected linking groups may be coupled to a resin and then aryl-halide may be attached by replacing the protecting group. Subsequently chelator groups may be reacted to the linker group to yield the compounds.

Such compounds of Table 5 suitably may comprise a radioisotope agent that may be complexed with the compound. For instance, a compound as set forth in Table 5 may comprise (e.g. complexed with) one or more of ¹⁷⁷Lu, ²²⁵Ac, ⁶⁷Cu, ¹⁶¹Tb, ⁶⁷Ga, ²⁰³Pb, ²¹²Pb and/or ²²³Ra.

Also preferred are pharmaceutical compositions that comprise one or more compounds set forth in Table 5, including where such compound(s) are complexed with one or more radioisotope agents such as e.g. one or more ¹⁷⁷Lu, ²²⁵Ac, ¹⁶¹Tb, ⁶⁷Ga, ²⁰³Pb, ²¹²Pb and ²²³Ra. Preferred pharmaceutical compositions that comprise one or more compounds set forth in Table 5 may further comprise one or more gentisate compounds. Preferred pharmaceutical compositions that comprise one or more compounds set forth in Table 5 include those that maintain a radiochemical purity of 95% or greater as determined by radio-HPLC after 3 days at 30° C.

In one preferred aspect of the specification, the radiopharmaceutical agent comprises one of the compounds including ²¹²Pb or ²⁰³Pb as shown in the following Table 6. To provide a compound of Table 6, a corresponding compound in Table 5 may be suitably complexed with ²¹²Pb or ²⁰³Pb in similar process as described herein, e.g., Example 1.

TABLE 6

| Compounds | Structure |
|---|---|
| [Pb-212]Pb-TCMC- PSMA I&T MW: 1635.36 Formula: C₆₀H₉₁IN₁₄O₁₈²¹²Pb | 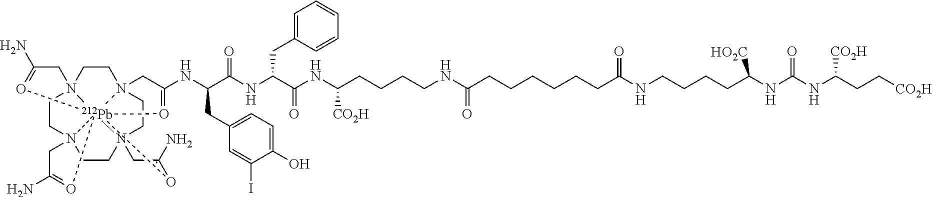 |
| [Pb-203]Pb-TCMC- PSMA I&T Formula: C₆₀H₉₁IN₁₄O₁₈²⁰³Pb | 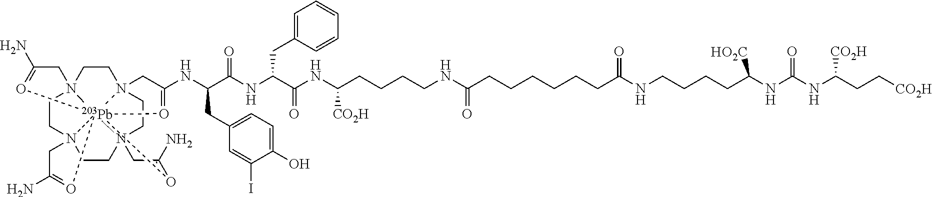 |
| [Pb-212]Pb-p-SCN- Bn-TCMC-PSMA I&T MW: 1799.59 Formula: C₆₈H₉₉IN₁₆O₁₈S²¹²Pb | 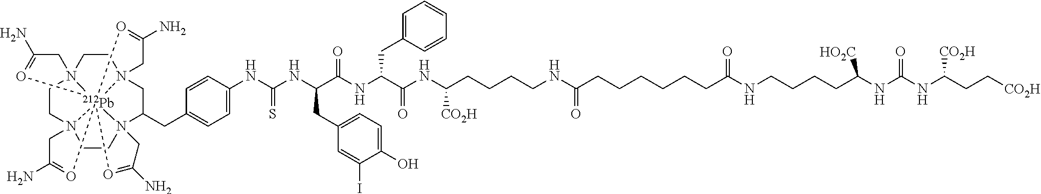 |
| [Pb-203]Pb-p-SCN- Bn-TCMC-PSMA I&T Formula: C₆₈H₉₉IN₁₆O₁₈S²⁰³Pb | 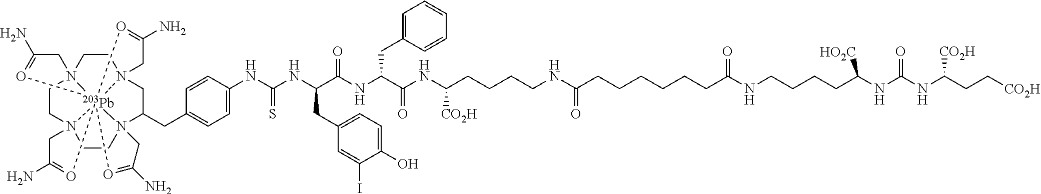 |
| [Pb-212]Pb-PSC- PSMA I&T MW: 1635.32 Formula: C₆₀H₈₉IN₁₂O₂₀²¹²Pb | 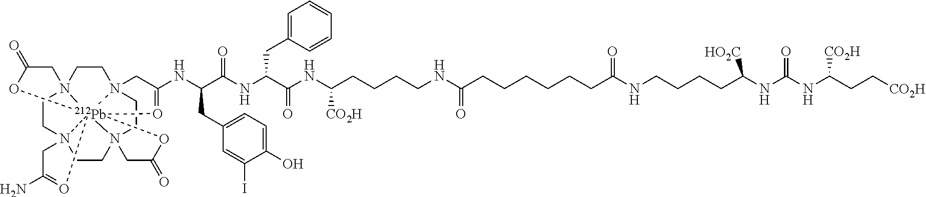 |
| [Pb-203]Pb-PSC- PSMA I&T Formula: C₆₀H₈₉IN₁₂O₂₀²⁰³Pb | 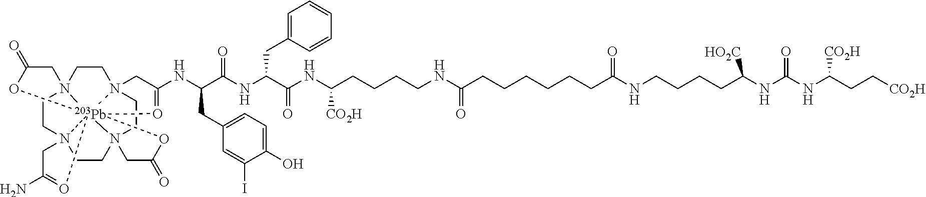 |

Also preferred are pharmaceutical compositions that comprise one or more compounds set forth in Table 6. Preferred pharmaceutical compositions that comprise one or more compounds set forth in Table 6 may further comprise one or more gentisate compounds. Preferred pharmaceutical compositions that comprise one or more compounds set forth in Table 6 include those that maintain a radiochemical purity of 95% or greater as determined by radio-HPLC after 3 days at 30° C.

In certain aspects, complexes of PSMA I & T (EuK-Sub-kf-iodo-y-DOTAGA) are preferred radiopharmaceutical compounds, including ²¹²Pb-PSMA I & T, ¹⁶¹Tb-PSMA I & T, ²²⁵Ac-PSMA I & T and ⁶⁷Cu-PSMA I & T. The term “complex” herein generally refers to a union of the radionucleotide (e.g. ¹⁷⁷Lu, ²¹²Pb, ¹⁶¹Tb, ²²⁵Ac, ⁶⁷Cu) and the complexing ligand such as EuK-Sub-kf-iodo-y-DOTAGA inclusive of chemical and physical variations that may exist, such as in the linker or chelator, when utilized with a radionucleotide other than lutetium-177.

EuK-Sub-kf-iodo-y-DOTAGA and complexed compounds thereof each has several possible stereoisomers, including the R and S isomers of the carbon that is an N-ring substituent of the tetraazacyclotetradecane moiety of those compounds. References herein to EuK-Sub-kf-iodo-y-DOTAGA and radioisotope-complexed compounds thereof (including ²¹²Pb-PSMA I & T, ¹⁶¹Tb-PSMA I & T, ²²⁵Ac-PSMA I & T, and ⁶⁷Cu-PSMA I & T) without further limitation includes all possible stereoisomers of each of those compounds and particularly both the noted R and S isomers.

In certain aspects, racemic mixtures of EuK-Sub-kf-iodo-y-DOTAGA and radioisotope-complexed compounds thereof (including ²¹²Pb-PSMA I & T, ¹⁶¹Tb-PSMA I & T, ²²⁵Ac-PSMA I & T and ⁶⁷Cu-PSMA I & T) are provided, including for use in the present pharmaceutical compositions and methods.

In other preferred aspects, optically enriched mixtures of EuK-Sub-kf-iodo-y-DOTAGA and radioisotope-complexed compounds thereof (including ²¹²Pb-PSMA I & T, ¹⁶¹Tb-PSMA I & T, ²²⁵Ac-PSMA I & T and ⁶⁷Cu-PSMA I & T) are provided, including for use in the present pharmaceutical compositions and methods.

In a preferred aspect, R isomer-enriched EuK-Sub-kf-iodo-y-DOTAGA is provided, i.e. EuK-Sub-kf-iodo-y-DOTAGA that is comprised of a weight excess of the R isomer (referring to the *carbon having N-ring substitution) of the following structure 1A:

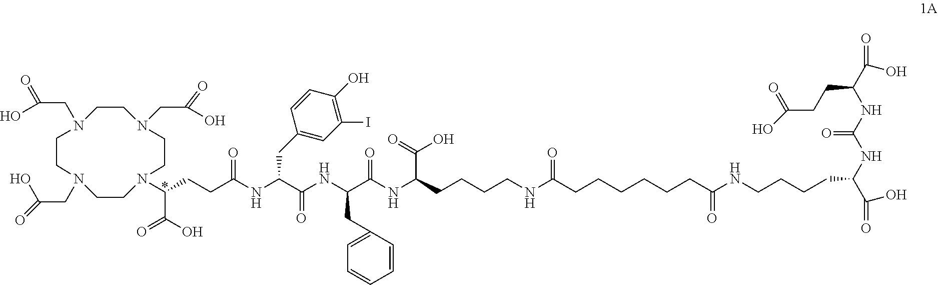 [SMILES: O=C(O)CC[C@H](NC(=O)N[C@@H](CCCCNC(=O)CCCCCCC(=O)NCCCC[C@@H](NC(=O)[C@@H](Cc1ccccc1)NC(=O)[C@@H](Cc1ccc(O)c(I)c1)NC(=O)CC[C@H](C(=O)O)N1CCN(CC(=O)O)CCN(CC(=O)O)CCN(CC(=O)O)CC1)C(=O)O)C(=O)O)C(=O)O]

In this aspect, generally preferred is EuK-Sub-kf-iodo-y-DOTAGA that is substantially optically enriched with the R isomer of structure 1A, or is an enantiomerically pure mixture of the R isomer of structure 1A.

In another aspect, S isomer-enriched EuK-Sub-kf-iodo-y-DOTAGA is provided, i.e. EuK-Sub-kf-iodo-y-DOTAGA that is comprised of a weight excess of the S isomer (referring to the *carbon having N-ring substitution) of the following structure 1B:

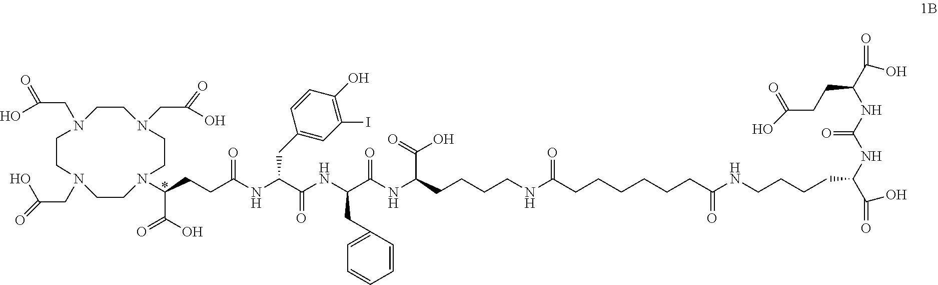 [SMILES: O=C(O)CC[C@H](NC(=O)N[C@@H](CCCCNC(=O)CCCCCCC(=O)NCCCC[C@@H](NC(=O)[C@@H](Cc1ccccc1)NC(=O)[C@@H](Cc1ccc(O)c(I)c1)NC(=O)CC[C@@H](C(=O)O)N1CCN(CC(=O)O)CCN(CC(=O)O)CCN(CC(=O)O)CC1)C(=O)O)C(=O)O)C(=O)O]

In such aspect, generally preferred is EuK-Sub-kf-iodo-y-DOTAGA that is substantially optically enriched with the S isomer of structure 1B, or is an enantiomerically pure mixture of the S isomer of structure 1B.

Additionally, in certain preferred aspects, the R of PSMA I & T is provided, including for use in the present pharmaceutical compositions and methods. That R isomer may be represented by the following structure 2A-2, 2A-3, and 2A-4:

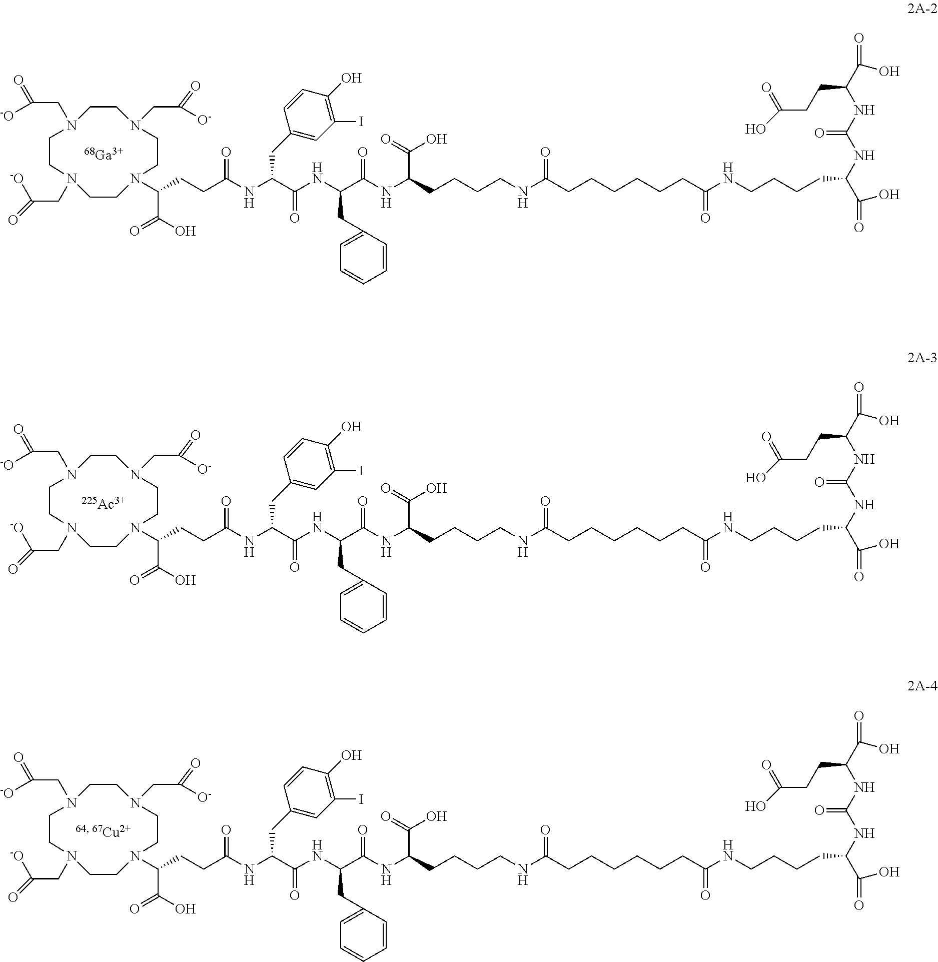

In this aspect, generally preferred compounds are substantially optically enriched with the R isomer of structure 2A-1, 2A-2, 2A-3, and 2A-4, or is an enantiomerically pure mixture of the R isomer of structure 2A-1, 2A-2, 2A-3, and 2A-4.

In another aspect, the S isomer of PSMA I & T is provided, including for use in the present pharmaceutical compositions and methods. That S isomer may be represented by the following structure 2B-2, 2B-3 and 2B-4:

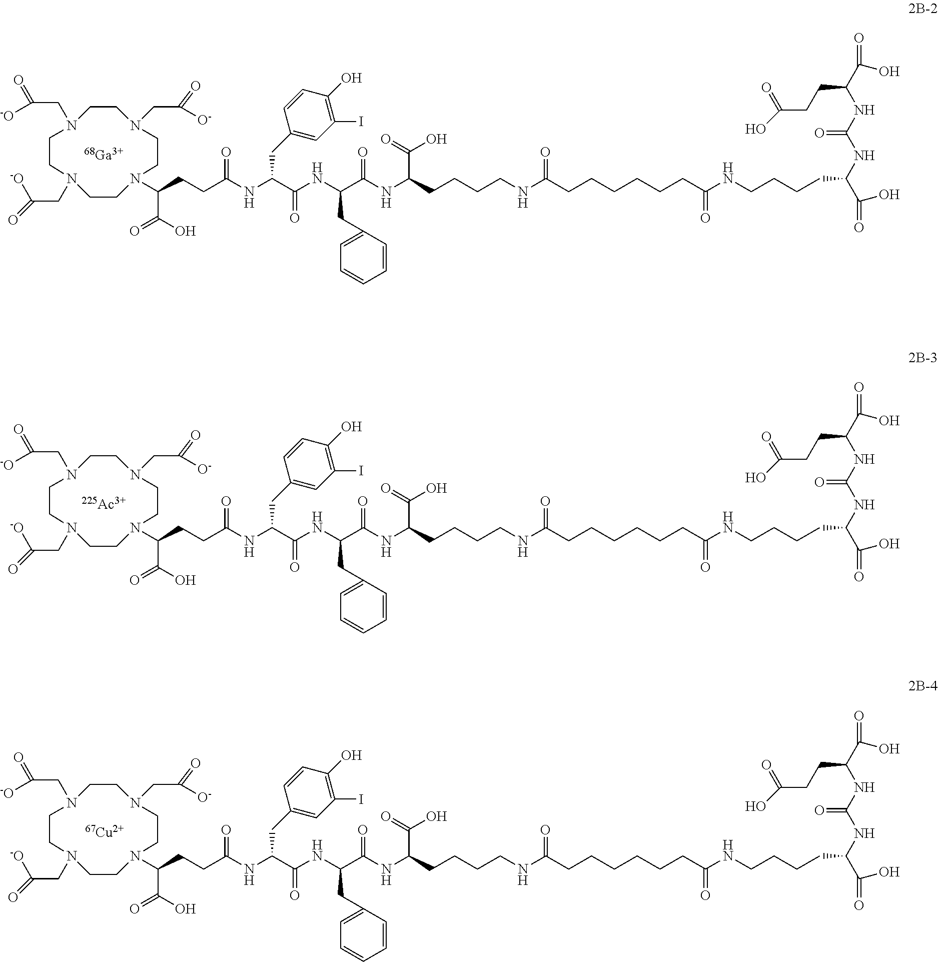

In this aspect, generally preferred compounds are PSMA I & T that are substantially optically enriched with the S isomer of structure 2B-1, 2B-2, 2B-3, and 2B-4, or is an enantiomerically pure mixture of the S isomer of structure 2B-1, 2B-2, 2B-3, and 2B-4.

The present PSMA I & T compounds and radioisotope-complexed compounds thereof (including ²¹²Pb-PSMA I & T, ¹⁶¹Tb-PSMA I & T, ²²⁵Ac-PSMA I & T and ⁶⁷Cu-PSMA I & T) can exhibit particularly favorable chemical or radiochemical purities, including greater than 95, 96, 97, 98, 99, 99.5, 99.6, 99.7, 99.8 or 99.9 percent radiochemical purity and/or substantial absence of one or more prior impurities.

In preferred aspects, a complex of PSMA I & T such as ²¹²Pb-PSMA I & T, ¹⁶¹Tb-PSMA I & T, ²²⁵Ac-PSMA I & T and ⁶⁷Cu-PSMA I & T is provided in methods that comprise admixing the corresponding radioisotope (Lead-212, Copper-67, Terbium-161 and/or Actinium-225) and EuK-Sub-kf-iodo-y-DOTAGA in the complete or substantial absence of one or more gentisate compounds.

Applicant has found that at least substantial absence of any gentisate compounds during the incorporation reaction (including heating) can substantially reduce impurities that would otherwise be produced through a radioisotope incorporation reaction. See, for instance, Examples 1 and 2 which follow.

As referred to herein, “the incorporation reaction” or similar term refers to the reaction to incorporate (e.g. complex) a radionuclide such as Lutetium-177, Copper-67, Lead-212, Terbium-161 and/or Actinium-225 with the associated complexing molecule such as EuK-Sub-kf-iodo-y-DOTAGA to thereby produce the radiopharmaceutical compound such as ¹⁷⁷Lu-PSMA I & T. In certain aspects, the incorporation reaction may include admixing lutetium-177 or other radioisotope with EuK-Sub-kf-iodo-y-DOTAGA and heating the radioisotope/EuK-Sub-kf-iodo-y-DOTAGA admixture.

Methods also are provided for preparing radioisotope-complexed PSMA I & T (including ¹⁷⁷Lu-PSMA I & T, ²²⁵Ac-PSMA I & T and ⁶⁷Cu-PSMA I & T) that include admixing the corresponding radioisotope (Lutetium-177, Copper-67, Lead-212, Terbium-161 and/or Actinium-225) and EuK-Sub-kf-iodo-y-DOTAGA in the presence of one or more ascorbate compounds suitably in an aqueous composition. The one or more ascorbate compounds may be referred to as a component of the “Reaction Composition” or similar term when added to or otherwise present with either the radioisotope or EuK-Sub-kf-iodo-y-DOTAGA as part of the reaction to incorporate (e.g. complex) the radioisotope with EuK-Sub-kf-iodo-y-DOTAGA to prepare the complexed PSMA I & T (such as ¹⁷⁷Lu-PSMA I & T, ²²⁵Ac-PSMA I & T, and ⁶⁷Cu-PSMA I & T). The Reaction Composition is suitably an aqueous composition.

The Reaction Composition may comprise one or more other agents, particularly one or more distinct organic compounds in addition to ascorbate compound(s). Such additional distinct organic compounds sometimes are referred to herein as “stabilizer compounds.” The term “stabilizer compound” or “stabilizer compounds” includes one or more ascorbate compounds.

In particular aspects, the EuK-Sub-kf-iodo-y-DOTAGA precursor (structure 1A and/or 1B) may be diluted before admixture with a radioisotope compound, such as diluting a compound of structure 1A and/or 1B with an aqueous composition preferably comprising one or more ascorbate compounds.

It has been found that the presence of one or more ascorbate compounds during the incorporation reaction (including heating) can substantially reduce impurities that would otherwise be produced through the radioisotope incorporation.

In particular, it has been found that the presence of one or more ascorbate compounds during the incorporation reaction (including heating) can substantially reduce impurities that would otherwise be produced through the radioisotope incorporation in the absence of a radioprotectant or in the presence of gentisic acid, for example where a gentisate adduct is formed with radioisotope-complexed PSMA I & T.

In preferred systems, one or more ascorbate compounds are present during the incorporation reaction (including heating) together with at least substantial absence or preferably complete absence of one or more gentisate compounds during that incorporation reaction to reduce impurities that would otherwise be produced through radioisotope incorporation. In such an aspect, gentisate compound(s) would be substantially absent (or an admixture comprising a radioisotope compound, EuK-Sub-kf-iodo-y-DOTAGA and one or more ascorbate compounds would be substantially free of gentisate compound(s)) if one or more gentisate compounds were present in an amount of less than 10, 8, 5, 4, 3, 2, 1 or 0.5 weight percent relative to the weight amount of one or more ascorbate compounds present during an incorporation reaction.

It has been found that a substantial or complete absence of one or more gentisate compounds during the incorporation reaction can reduce or avoid the occurrence or formation of an impurity that has been detected by high-performance liquid chromatography (HPLC).

The impurity or impurities that has been observed upon use of one or more gentisate compounds such as gentisic acid during the incorporation reaction to form a radioisotope-complex of PSMA I & T is sometimes referred to herein as “gentisate adduct impurity”.

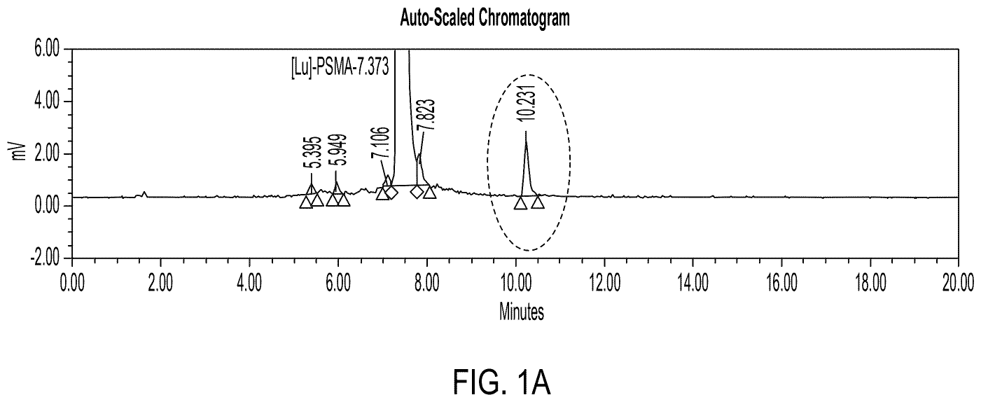

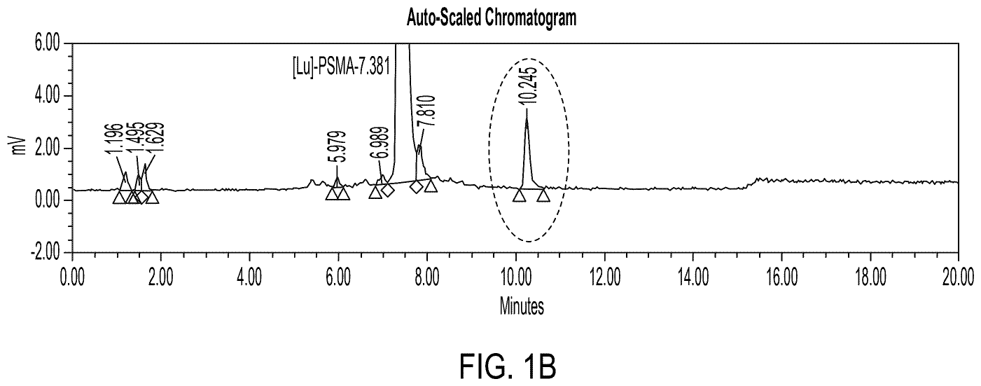

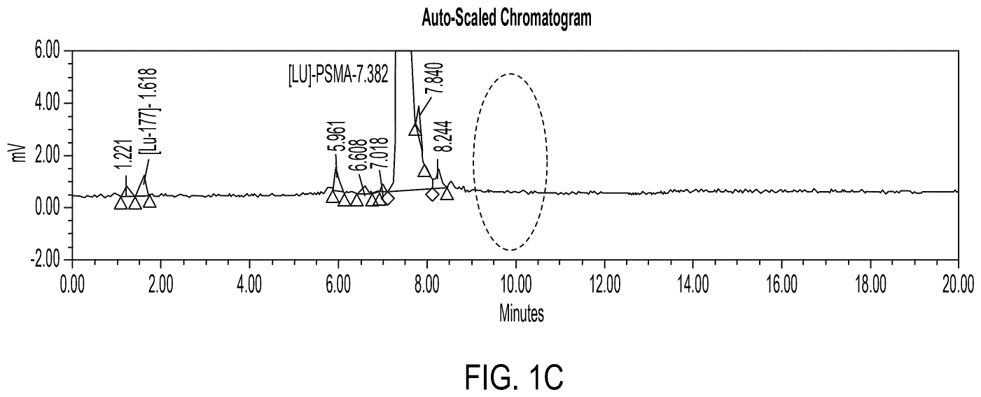

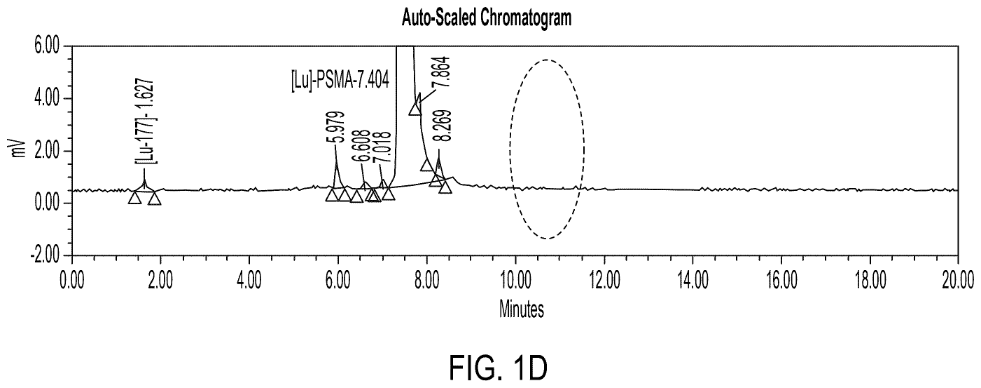

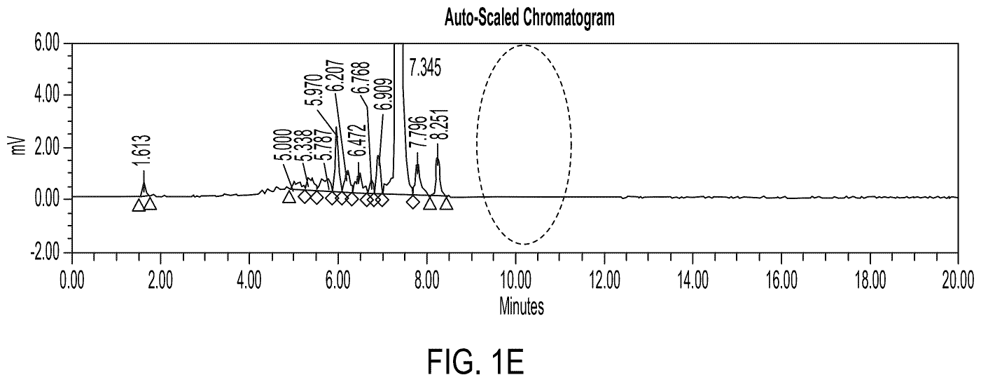

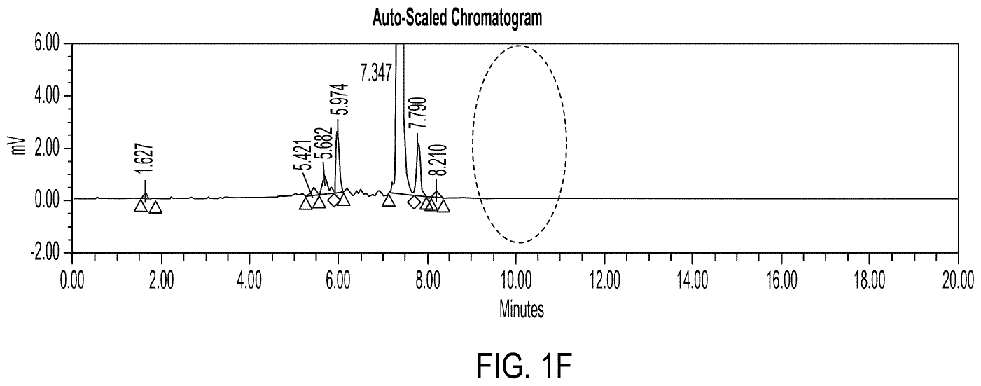

In one aspect, a “gentisate adduct impurity” as referred to herein has been characterized as have a retention time in the region of 10.2 minutes by high-performance liquid chromatography (HPLC) with a by high-performance liquid chromatography (HPLC) with a Waters XBridge BEH Phenyl-Hexyl Column, 130 Å, 3.5 μm, 4.6 mm×150 mm using 0.1% trifluoracetic acid in water (Mobile Phase A) and 0.1% trifluoracetic acid in acetonitrile. A linear gradient from 85% Mobile Phase A to 55% Mobile Phase A over 12 minutes is used and the ratio is held for 15 minutes. The absence of such impurity would be demonstrated by a no peaks (e.g. by visible review of spectra) being present in the 9 to 12 minute region of an HPLC chromatogram corresponding, in particular aspects the 9 or 9.5 to 10.5 or 11 minute region or the about 10.2 minute region as exemplified by the radio chromatograms of FIGS. 1C and 1D.

In another aspect, a “gentisate adduct impurity” as referred to herein may be characterized as a compound that include a covalent linkage of 1) a gentisate compound such as gentisic acid or reaction product or other derivative of such gentisate compound and 2) EuK-Sub-kf-iodo-y-DOTAGA.

For instance, without being bound by any theory, gentisic acid oxidation can result in formation of a benzoquinone as shown in the following Scheme 1.

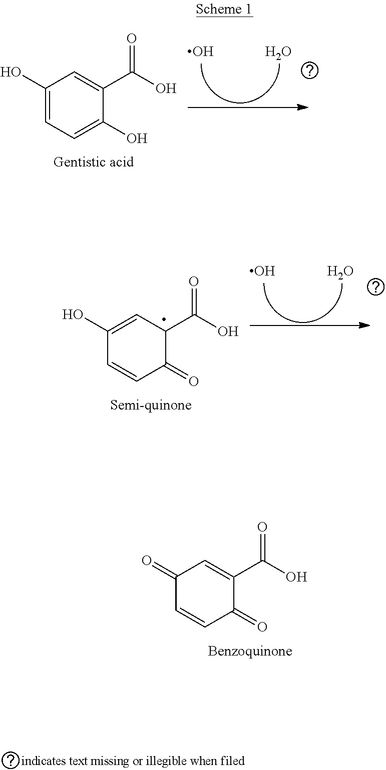

That benzoquinone then may covalently couple with EuK-Sub-kf-iodo-y-DOTAGA during the incorporation reaction, for example to produce Adduct Compound Structures A1 and/or A2 (mass 1803 g/mol) as gentisate adduct impurities.

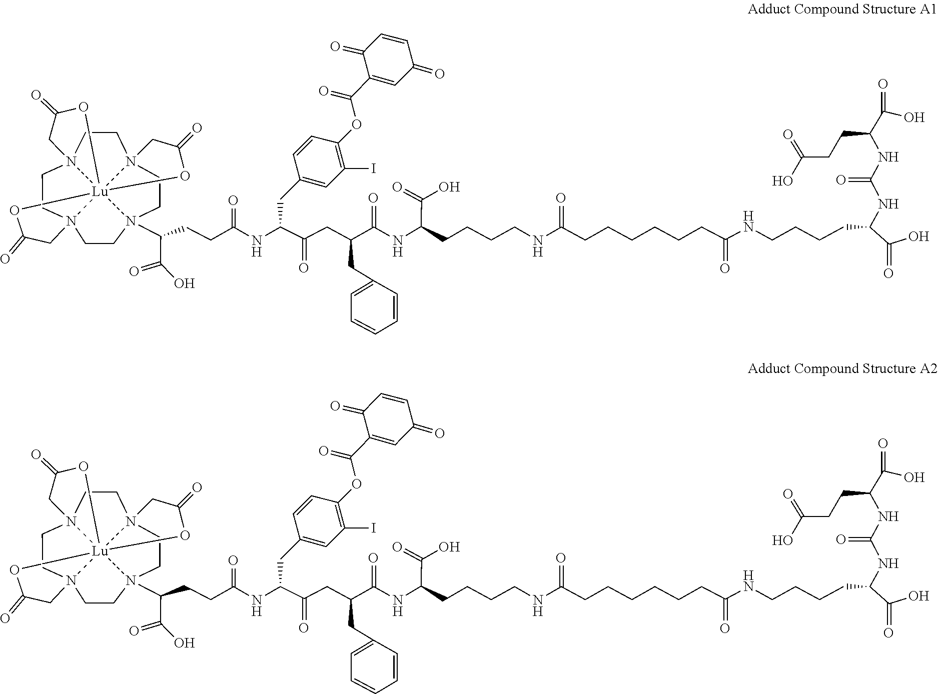

In a further aspect, suitably a composition (sometimes referred herein as the Formulation Composition and distinct from the Reaction Composition) is added following or upon termination of heating of the radioisotope and EuK-Sub-kf-iodo-y-DOTAGA or other complexing compound particularly where a complex of radioisotope compound and EuK-Sub-kf-iodo-y-DOTAGA or other complexing compound has been formed.

In particular, we have found enhanced stability and shelf-life of the formed radioisotope-complexed PSMA I & T can be achieved by treatment of the incorporation reaction composition with one or more gentisate compounds following or upon termination of the reaction heating step.

Thus, methods are providing for preparing a radioisotope-complex of PSMA I & T, comprising a) admixing 1) a radioisotope such as Lutetium-177, Copper-67, Lead-212, Terbium-161 and/or Actinium-225 Lutetium-177, Copper-67, Lead-212, Terbium-161 and/or Actinium-225 and 2) EuK-Sub-kf-iodo-y-DOTAGA; b) heating the admixed 1) radioisotope and 2) EuK-Sub-kf-iodo-y-DOTAGA, wherein a complex of the radioisotope and EuK-Sub-kf-iodo-y-DOTAGA is formed; and c) adding one or more gentisate compounds while or after the heating is reduced or terminated. In certain aspects, both 1) one or more gentisate compounds and 2) one or more ascorbate compounds are added while or after the heating is reduced or terminated.

In a particular preferred embodiment, a gentisate compound is added to the formed complex of a radiopharmaceutical compound such as complex of a radioisotope and EuK-Sub-kf-iodo-y-DOTAGA promptly after the termination of the heating step, for example within 0.25, 0.5, 1 or 2 minutes of initiation of termination (complete removal of heating source, or the occurrence of an at least 20° C. temperature drop) of the radiopharmaceutical compound (e.g. lutetium-177 and EuK-Sub-kf-iodo-y-DOTAGA) incorporation reaction.

In yet a further preferred embodiment, both 1) a gentisate compound and 2) an ascorbate compound are added to the formed radiopharmaceutical compound such as formed complex of the radioisotope and EuK-Sub-kf-iodo-y-DOTAGA promptly after the termination of the heating step, for example within 0.25, 0.5, 1 or 2 minutes of initiation of termination (complete removal of heating source, or the occurrence of an at least 20° C. temperature drop) of the radiopharmaceutical compound incorporation reaction

Such a post-heating Formulation Composition may be an aqueous composition comprising one or more gentisate compounds and preferably one or more ascorbate compounds.

In certain aspects, following the radioisotope incorporation reaction, the produced radioisotope-complex of PSMA I & T (e.g. ¹⁷⁷Lu-PSMA I & T, ²²⁵Ac-PSMA I & T and ⁶⁷Cu-PSMA I & T) may be first treated with one or more ascorbate compounds in the absence of a gentisate compound and subsequent to such ascorbate compound treatment the radioisotope-complex of PSMA I & T may be treated (e.g. admixed) with one or more gentisate compounds.

In other aspects, following the radioisotope incorporation reaction, the produced PSMA I & T complex may be treated substantially simultaneously with one or more ascorbate compounds and one or more gentisate compounds. For instance, an aqueous formulation comprising both ascorbic acid or salt thereof or other ascorbate compound and gentisic acid or salt thereof or other gentisate compound may be added to the formed complex of the radioisotope and EuK-Sub-kf-iodo-y-DOTAGA or other complexing compound promptly after the termination of the heating step.

In yet additional aspects, following the radioisotope incorporation reaction, the produced radioisotope-complex of PSMA I & T (e.g. ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T) may be first treated with one or more gentisate compounds in the absence of an ascorbate compound and subsequent to such gentisate compound treatment the radioisotope-complex of PSMA I & T T (e.g. ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T) may be treated (e.g. admixed) with one or more ascorbate compounds.

The aqueous admixture of the radiopharmaceutical composition such as ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T and one or more gentisate compounds and optionally one or more ascorbate compounds can be stored until administration to a patient.

It has been found that such post-incorporation reaction use of one or more ascorbate compounds in combination with one or more gentisate compounds can provide enhanced stability and shelf life of the radiopharmaceutical composition such as a composition comprising ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T. See, for instance, Examples 1 and which follow.

As referred to herein, an ascorbate compound or composition suitably may include for example ascorbic acid and/or an ascorbate salt such as sodium L-ascorbate, among others.

A gentisate compound or composition as referred to herein includes for example gentisic acid (2,5-dihydroxybenzoic acid). The term gentisate compound or composition also includes salts and esters of gentisic acid. A variety of gentisic acid salts may be suitably utilized as disclosed herein including for instance alkali metal, alkaline earth metal, and ammonium salts. Sodium and potassium salts may be preferred in some aspects. Ester compounds also may be utilized in certain aspects including for instance compounds esterified at one or both of the gentisic acid hydroxyl groups, such as compounds that have the 2-hydroxyl and/or 5-hydroxyl moieties functionalized with a methyl ester, ethyl ester or other C₁₋₆ alkyl esters. In at least certain aspects, preferred gentisate compounds or compositions include gentisic acid or a gentisic acid salt.

In preferred aspects, the incorporation of the radioisotope compound and EuK-Sub-kf-iodo-y-DOTAGA includes at least one of the following 1), 2, 3) or 4) and preferably at least 1) and 2), more preferably at least 1), 2) and 3) and still more preferably each of the following 1), 2), 3) and 4):
1) inclusion of one or more ascorbate compound in the radioisotope incorporation reaction mixture. Optionally one or more gentisate compounds also may be present in the radioisotope reaction mixture, but such inclusion of gentisate compound is less preferred as discussed. Thus, particularly preferred is inclusion of one or more ascorbate compound in the radioisotope incorporation reaction mixture and without the inclusion of a gentisate compound;
2) a short heat treatment such up to or less than 30, 25, 20, 15, 12, 10 or 8 minutes as described above;
3) heating temperatures of the incorporation reaction mixture such as up to or less than 40° C., 45° C., 50° C., 55° C., 60° C., 65° C., 70° C., 75° C., 80° C., 85° C., 90° C. or 95° C.; and
4) a post-heating treatment (addition) with one or more gentisate compounds and/or one or more ascorbate compounds and preferably one or more gentisate compounds.

Preferred pharmaceutical compositions include aqueous compositions that include 1) a complex of a radioisotope compound and EuK-Sub-kf-iodo-y-DOTAGA (e.g. ¹⁷⁷Lu-PSMA I & T, ²¹²Pb-PSMA I & T, ¹⁶¹Tb-PSMA I & T, ²²⁵Ac-PSMA I & T or ⁶⁷Cu-PSMA I & T) and 2) i) one or more ascorbate compounds and ii) one or more gentisate compounds.

The one or more ascorbate compounds may be suitably present in a pharmaceutical composition in varying amounts, such as 5 or 10 mg/mL to 120 mg/mL; or 30 mg/mL to 100 mg/mL; or 40 mg/mL to 80 or 90 mg/mL. In a particular preferred aspect, the one or more ascorbate compounds such as an ascorbate salt may be present in an amount of 55 mg/mL to 75 mg/mL.

As discussed, the one or more ascorbate compounds may be incorporated both as a component of the radioisotope and EuK-Sub-kf-iodo-y-DOTAGA incorporation reaction as well as once the radioisotope/EuK-Sub-kf-iodo-y-DOTAGA complex has been formed.

In such multiple additions to provide a desired amount of ascorbate compound(s) in a final pharmaceutical composition (e.g. pharmaceutical composition comprising ¹⁷⁷Lu-PSMA I & T, ²¹²Pb-PSMA I & T, ¹⁶¹Tb-PSMA I & T, ²²⁵Ac-PSMA I & T or ⁶⁷Cu-PSMA I & T), a Reaction Composition may contain one or more ascorbate compounds such as ascorbic acid and/or an ascorbate salt in an amount of 5 or 10 mg/mL to 120 mg/mL; or 5 or 10 mg/mL to 100 or 110 mg/mL; or 5 or 10 mg/mL to 80 or 90 mg/mL, in a particular preferred aspect, the one or more ascorbate compounds such as ascorbic acid and/or an ascorbate salt may be present in an amount of 55 mg/mL to 75 mg/mL.

In the present pharmaceutical compositions one or more gentisate compounds such as gentisic acid or a gentisate salt may be present in an amount of for example 5 or 10 mg/mL to 100 mg/mL; or 5 or 10 mg/mL to 60 or 80 mg/mL; or 5 or 10 mg/mL to 40 or 50 mg/mL. In a particular preferred aspect, the one or more gentisate compounds such as a gentisate salt may be present in an amount of 16 mg/mL to 36 mg/mL. As discussed, such amounts of one or more gentisate compounds may be preferably added to a formed radiopharmaceutical compound such as a formed complex of the radioisotope compound and a complexing compound such as EuK-Sub-kf-iodo-y-DOTAGA, TCMC, PSC while or after heating, in the heating step of the incorporation reaction, is reduced or terminated. As also discussed, the one or more gentisate compounds may be preferably added to a formed radiopharmaceutical compound such as a formed complex of lutetium-177 and the complexing compound such as EuK-Sub-kf-iodo-y-DOTAGA in combination with an ascorbate compound that is either already present with the complex of lutetium-177 and the complexing compound such as EuK-Sub-kf-iodo-y-DOTAGA, and/or a combination of a gentisate compound and an ascorbate compound are added in combination to a formed radiopharmaceutical compound such as a formed complex of lutetium-177 and EuK-Sub-kf-iodo-y-DOTAGA. In certain preferred aspect, the one or more gentisate compounds such as gentisic acid may be present in amounts up to 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL or 100 mg/mL or more, in such formulation containing a formed radiopharmaceutical compound such as a formed complex of a radioisotope and EuK-Sub-kf-iodo-y-DOTAGA.

Additionally, one or more ascorbate compounds such as ascorbic acid and/or an ascorbate salt may be added to a formed radiopharmaceutical compound such as a formed complex of lutetium-177 and a complexing compound such as EuK-Sub-kf-iodo-y-DOTAGA or other agent to provide a pharmaceutical formulation containing the formed radiopharmaceutical compound wherein a total amount of ascorbate compound(s) is for example 5 or 10 mg/mL to 100 mg/ml in the formulation; or 5 or 10 mg/mL to 60 or 80 mg/mL in the formulation; or 5 or 10 mg/mL to 40 or 50 mg/mL in the formulation. In a particular preferred aspect, the one or more ascorbate compounds such as ascorbic acid and/or an ascorbate salt may be present in an amount of 55 mg/mL to 75 mg/mL in such formulation containing the formed radiopharmaceutical compound such as a formed complex of lutetium-177 and a complexing compound such as EuK-Sub-kf-iodo-y-DOTAGA. In a further preferred aspect, the one or more ascorbate compounds such as ascorbic acid and/or an ascorbate salt may be present in lower amounts of amount of 10 mg/mL or 15 mg/mL to 20 mg/mL, 25 mg/mL, 30 mg/mL, 40 mg/mL, 45 mg/mL, 50 mg/mL, or 55 mg/mL, in such formulation containing the formed radiopharmaceutical compound such as a formed complex of lutetium-177 and EuK-Sub-kf-iodo-y-DOTAGA. In certain preferred aspect, the one or more ascorbate compounds such as ascorbic acid and/or an ascorbate salt may be present in amounts up to 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL or 100 mg/mL or more in such formulation containing the formed radiopharmaceutical compound such as a formed complex of a radioisotope and EuK-Sub-kf-iodo-y-DOTAGA or other complexing compound.

In the present pharmaceutical compositions, a radiopharmaceutical compound such as a complex of PSMAI & T or other chelator such as TCMC or PSC and a radioisotope also may be present in varying concentrations, such as to provide a volumetric radioactivity of at least 100 MBq/mL, preferably of at least 250, 300, 400, 500, 600, 700, 800, 900, 1000, 1200, 1400, 1600, 1800 or 2000 MBq/mL for 0.5, 1, 2, 3, 4 or 5 days following preparation of the pharmaceutical composition.

Preferably, the radiochemical purity of a pharmaceutical composition is at least 95%, 96%, 97%, 98% or 99% for 3, 4 or 5 days or more at a temperature of 30° C. or less following preparation of the composition. Even more preferably, the radiochemical purity of a pharmaceutical composition is at least 96%, 97% or 98% for 3, 4 or 5 days or more at a temperature of 30° C. or less following preparation of the composition.

In a further aspect, a complex of a radioisotope and a complexing compound such as EuK-Sub-kf-iodo-y-DOTAGA, TCMC, PSC or other complexing compound (e.g. ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T) wherein the radioisotope complex is:
1) free of unchelated lutetium-177, 225Ac and/or ⁶⁷Cu in an amount of not more than 2, 1.5, 1.0 or 0.5 weight % based on total weight of the radioisotope complex, such as may be determined by radiometric detection (including HPLC radiometric detection); and/or
2) free of radiochemical impurities in an amount of not more than 5, 4, 3.5, 3, 2.5, 2, 1.5, 1.0 or 0.5 weight % based on total weight of the radioisotope complex, such as may be determined by radiometric detection (including HPLC radiometric detection); and/or
3) free of chemical impurities in an amount of not more than 5, 4, 3, 2, 1 or 0.5 weight %, all weight % based on total weight of the radioisotope complex, such as may be determined by HPLC/UV analysis, and where the ¹⁷⁷Lu-PSMA I & T is maintained at 30° C. or less and such purity levels are exhibited for 3, 4 or 5 days or more following preparation of the radioisotope complex.

In a further aspect, a complex of a radioisotope and a complexing compound such as EuK-Sub-kf-iodo-y-DOTAGA, TCMC, PSC or other complexing compound (“radioisotope complex”) is provided, (e.g. ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T), wherein the radioisotope complex is:
1) free of unchelated lutetium-177, ²²⁵Ac, ²¹²Pb ¹⁶¹Tb and/or ⁶⁷Cu in an amount of not more than 2, 1.5, 1.0 or 0.5 weight % based on total weight of the radioisotope complex, such as may be determined by radiometric detection (including HPLC radiometric detection); and/or
2) free of radiochemical impurities in an amount of not more than 5, 4, 3.5, 3, 2.5, 2, 1.5, 1.0 or 0.5 weight % based on total weight of the radioisotope complex, such as may be determined by radiometric detection (including HPLC radiometric detection); and/or
3) free of chemical impurities including a gentisate adduct impurity in an amount of not more than 5, 4, 3, 2, 1 or 0.5 weight %, all weight % based on total weight of the radioisotope complex, such as may be determined by HPLC/UV analysis, and where the radioisotope complex is maintained at 30° C. or less and such purity levels are exhibited for 3, 4 or 5 days or more following preparation of the radioisotope complex.

In further aspects, methods for preparing a radioisotope complex (e.g. ²¹²Pb-PSMA I & T, 161 Tb-PSMA I & T, ²²⁵Ac-PSMA I & T or ⁶⁷Cu-PSMA I & T, and/or ¹⁷⁷Lu-PSMA I & T) are provided that comprise: a) admixing 1) a radioisotope compound (such as lutetium-177, ²¹²Pb, ²²⁵Ac, ¹⁶¹Tb and/or ⁶⁷Cu) and 2) EuK-Sub-kf-iodo-y-DOTAGA or other complexing compound; and b) heating the admixed 1) radioisotope compound and 2) the complexing compound such as EuK-Sub-kf-iodo-y-DOTAGA for 15 minutes or less; wherein a radioisotope complex is formed.

Preferably the admixed 1) radioisotope compound and 2) the complexing compound such as EuK-Sub-kf-iodo-y-DOTAGA are at least substantially free of a gentisate compound during the b) heating. Preferably, one or more ascorbate compounds are admixed with the 1) radioisotope compound and 2) the complexing compound such as EuK-Sub-kf-iodo-y-DOTAGA.

In further preferred aspects, the methods further comprise adding one or more gentisate compounds to a formed radiopharmaceutical compound such as a complex of a radioisotope compound and the complexing compound such as EuK-Sub-kf-iodo-y-DOTAGA, TCMC, PSC or other agent for example where such one more gentisate compounds are added upon reduction or termination of heat.

In still further preferred aspects, the methods may further comprise adding one or more ascorbate compounds to a formed radiopharmaceutical compound, for example where such one or more ascorbate compounds are added upon reduction or termination of heat.

Methods are also provided for preparing a radioisotope complex (e.g. ¹⁷⁷Lu-PSMA I & T, ²¹²Pb-PSMA I & T, ¹⁶¹Tb-PSMA I & T, ²²⁵Ac-PSMA I & T or ⁶⁷Cu-PSMA I & T), comprising a) admixing 1) a radioisotope compound (such as lutetium-177, ²¹²Pb, ¹⁶¹Th, ²²⁵Ac and/or ⁶⁷Cu) and 2) a complexing compound such as EuK-Sub-kf-iodo-y-DOTAGA, TCMC, PSC or other agent; b) heating the admixed 1) radioisotope compound and 2) the complexing compound, wherein a complex of radioisotope compound and the complexing compound is formed; and c) adding one or more gentisate compounds while or after the heating is reduced or terminated. In certain aspects, both 1) a gentisate compound and 2) an ascorbate compound are added while or after the heating is reduced or terminated. Suitably, an admixture of 1) the radioisotope compound, 2) the complexing compound such as EuK-Sub-kf-iodo-y-DOTAGA, TCMC, PSC or other agent, and 3) one or more stabilizers are heated, wherein a radioisotope complex is formed.

### Definitions

Unless otherwise specified, the below terms used herein are defined as follows:

As used herein, the term “a,” “an,” “the” and similar terms used in the context of the present invention (especially in the context of the claims) are to be construed to cover both the singular and plural unless otherwise indicated herein or clearly contradicted by the context.

The language “and/or” is used herein as a shorthand notation to represent the expression “and,” describing the combination of items, as well as “or,” describing the items in the alternative form.

The term “compound” when referring to a compound of this invention, refers to a collection or population of molecules having an identical chemical structure, except that there may be isotopic variation among the constituent atoms of the molecules.

Certain compounds of the present invention possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)-isomers, and individual isomers are encompassed within the scope of the present invention. The compounds of the present invention do not include those that are known in art to be too unstable to synthesize and/or isolate. The present invention is meant to include compounds in racemic and optically pure forms. Optically active (R)- and (S)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

Unless otherwise stated, structures depicted herein are also meant to include all stereochemical forms of the structure, i.e., the R and S configurations for each asymmetric center. Therefore, single stereochemical isomers as well as enantiomeric and diastereomeric mixtures of the present compounds are within the scope of the invention.

A “radioisotope”, “radionuclide” or “radioactive isotope” as used herein are used interchangeably and is a radioactive isotope of an element (included by the term “radionuclide”) emitting α-, β- or γ-radiation. Exemplary radioactive isotopes include for example, ¹⁸F, ⁴³K ⁴⁷Sc, ⁵¹Cr, ⁵⁷Co, ⁵⁸Co, ⁵⁹Fe, ⁶⁷Cu, ⁶⁷Ga, ⁷¹Ge, ⁷²As, ⁷²Se, ⁷⁵Br, ⁷⁶Br, ⁷⁷As, ⁷⁷Br, ⁸¹Rb, ⁸⁸Y, ⁹⁰Y, ⁹⁷Ru, ^{m99}Tc, ¹⁰⁰Pd, ^{101m}Rh, ¹⁰³Pb, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁴I, ¹²⁵I, ¹²⁷Cs, ¹²⁸Ba, ¹²⁹Cs, ¹³¹Cs, ¹³¹I, ¹³⁹La, ¹⁴⁰La, ¹⁴²Pr, ¹⁴³Pr, ¹⁴⁹Pm, ¹⁵¹Eu, ¹⁵³Eu, ¹⁵³Sm, ¹⁵⁹Gr, ¹⁶¹Tb, ¹⁶⁵Dy, ¹⁶⁶Ho, ¹⁶⁹Eu, ¹⁷⁵Yb, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁸Au, ¹⁹⁹Ag, ¹⁹⁹Au, ²⁰¹Tl, ²⁰³Pb, ²¹¹At, ²¹²Bi, ²¹²Pb, ²¹³Bi, ²²⁵Ac, ²²⁷Th, ⁴⁴Sc, ⁴⁷Sc, ⁷⁷As, ¹¹⁰In, ¹⁵²Tb, ¹⁴⁹Tb, ⁸⁶Y, ⁸³Sr, ⁸⁹Sr, ⁸⁹Zr, and ¹⁶⁶Dy. In certain aspects, preferred radioisotopes include ¹⁷⁷Lu, ²²⁵Ac, ¹⁶¹Tb, ²¹²Pb, ²²³Ra and ⁶⁷Cu.

The term “chelate” or “chelating agent” are used interchangeably in the context of the present invention and refer to a molecule, often an organic one, and often a Lewis base, having two or more unshared electron pairs available for donation to a metal ion. The metal ion is usually coordinated by two or more electron pairs to the chelating agent. The terms, “bidentate chelating agent”, tridentate chelating agent, and “tetradentate chelating agent” refer to chelating agents having, respectively, two, three, and four electron pairs readily available for simultaneous donation to a metal ion coordinated by the chelating agent. Usually, the electron pairs of a chelating agent forms coordinate bonds with a single metal ion; however, in certain examples, a chelating agent may form coordinate bonds with more than one metal ion, with a variety of binding modes being possible.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3).

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle.

The term “alkylene,” by itself or as part of another substituent, means, unless otherwise stated, a divalent radical derived from an alkyl, as exemplified, but not limited by, —CH₂CH₂CH₂CH₂-Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain.

The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. A “cycloalkylene” and a “heterocycloalkylene,” alone or as part of another substituent, means a divalent radical derived from a cycloalkyl and heterocycloalkyl, respectively.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring).

The term “substituted” means to contain or include at least one “substituent group”. For example, the “substituent group,” as used herein, means a group selected from oxo, halogen, —CF₃, —CCl₃, —CBr₃, —CI₃, —CHF₂, CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O) NHNH₂, —NHC(O) NH₂, —NHSO₂H, —NHC(O) H, —NHC(O) OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

The term “about”, as used herein, means an acceptable margin of error for a particular value, which depends in part on how the value is measured or determined. In certain embodiments, “about” as used herein will be understood by persons of ordinary skill in the art to mean up to plus or minus 20% of the particular term. In further embodiments, “about” as used herein will be understood by persons of ordinary skill in the art to mean up to plus or minus 10% of the particular term.

As used herein, the term “optically enriched” or “optical excess” denotes the presence of one or more non-racemic stereoisomeric centers in a molecule, wherein the configuration of at least one stereoisomeric center has a predominance of one stereoisomeric configuration (R or S). For example, one stereoisomeric center in a molecule, typically a carbon atom, may have greater than 50 or 55 weight % (based on total weight of the compound) of its attached atoms spatially arranged in the (R) configuration. Alternatively, more than 50 weight % (based on total weight of the compound) may be spatially arranged in the(S) configuration. More preferably the molecule, or its stereoisomeric center, is substantially optically enriched, and even more preferably is substantially enantiomerically pure.

As used herein, the term “substantially optically enriched” or “substantial optical excess”, when referring to a stereoisomer or stereoisomeric center, denotes that at least about 60 weight % (based on total weight of the compound), preferably about 70 weight % (based on total weight of the compound), more preferably about 80 weight % (based on total weight of the compound), still more preferably about 90 weight % (based on total weight of the compound) of one stereoisomer or one stereoisomeric center configuration predominates in the mixture, with at least about 95 weight % (based on total weight of the compound) of one stereoisomer or one stereoisomeric center configuration being even more preferred. In some preferred embodiments, the compound is “substantially enantiomerically pure”, that is, at least about 97.5 weight % (based on total weight of the compound), more preferably about 99 weight % (based on total weight of the compound), even more preferably about 99.5 weight % (based on total weight of the compound) of one stereoisomeric configuration predominates.

As used herein, the term “substantially pure” means sufficiently homogeneous to appear free of readily detectable impurities as determined by standard analytical methods, such as thin layer chromatography (TLC), gel electrophoresis, high performance liquid chromatography (HPLC), nuclear magnetic resonance (NMR), and mass spectrometry (MS); or sufficiently pure such that further purification would not detectably alter the physical and chemical properties, or biological and pharmacological properties, such as enzymatic and biological activities, of the substance. In certain embodiments, “substantially pure” refers to a collection of molecules, wherein at least about 55%, about 60%, about 65%, about 70%, about 75%, about 80%, about 85%, about 90%, about 95%, about 97%, about 98%, about 98.5%, about 99%, about 99.5% or about 99.9% or greater of the molecules are a single compound, including a racemic mixture or a single stereoisomer thereof, as determined by standard analytical methods.

As used herein, and unless otherwise specified, the terms “treat,” “treating” and “treatment” refer to the eradication or amelioration of a disease, disorder, or condition, or of one or more symptoms associated with the disease, disorder or condition. In certain embodiments, the terms refer to minimizing the advancement or worsening of the disease, disorder, or condition resulting from the administration of a formulation of the invention to a patient with such a disease, disorder, or condition. In some embodiments, the terms refer to the administration of a formulation provided herein, after the onset of symptoms of the particular disease, disorder, or condition. The terms “treat,” “treating”, “treatment”, or the like, as used herein covers the treatment of a disease, disorder, or condition in a subject, e.g., a mammal, and includes at least one of: (i) inhibiting the disease, disorder, or condition, i.e., partially or completely halting its progression; (ii) relieving the disease, disorder, or condition, i.e. causing regression of symptoms of the disease, disorder, or condition, or ameliorating a symptom of the disease, disorder, or condition; and (iii) reversal or regression of the disease, disorder, or condition, preferably eliminating or curing of the disease, disorder, or condition. In a particular embodiment the terms “treat,” “treating”, “treatment”, or the like, covers the treatment of a disease, disorder, or condition in a mammal, e.g., a primate, e.g., a human, and includes at least one of (i), (ii), and (iii) above. As is known in the art, adjustments for age, body weight, general health, sex, diet, time of administration, drug interaction and the severity of the condition may be necessary, and will be ascertainable with routine experimentation by one of ordinary skill in the art based on the invention described herein.

As used herein, the terms “subject”, and “patient” are used interchangeably. The terms “subject” and “patient” refer to an animal such as a mammal including non-primates (e.g., a cow, pig, horse, sheep, rabbit, guinea pig, rat, cat, dog, and mouse) and primates (e.g., a monkey, chimpanzee and a human). In a particular embodiment, the subject is a human.

As used herein, the term “shelf-life radioactive concentration” or “shelf-life RAC” means the radioactive concentration of a radiopharmaceutical formulation at or about the time of initial formulation to the concentration at which the formulation is intended to be shipped and stored, i.e., over a period of 3 or more days.

### Syntheses

Compounds as disclosed herein suitably may be prepared as disclosed in for example Brandt et al, Nuclear Medicine and Biology, 70 (2019) 46-52; Yao et al., Bioorganic & Medicinal Chemistry 28 (2020) 115319; Kelly et al., Journal of Nuclear Medicine vol. 58, no. 9 1442-Bioorganic & Medicinal Chemistry, 2017, 58 (9): 1442-1449; Benesova et al., Mol. Pharmaceutics, (2018) 15, 3, 934-946; Kuo et al., Mol. Pharmaceutics, (2018) 15, 11, 5183-5191; Kelly et al., J. Nucl Med 2019, 60:649-655; Davis, Pharmaceutics 2022, 14, 74; and Huynh et al., 2022 Pharmaceuticals, 15, 299.

¹⁷⁷Lu-PSMA I & T (including structures 2A-1, 2A-2, 2A-3, 2A-4, 2B-1, 2B-2, 2B-3, and 2B-4 below) can be prepared by complexing or incorporating ¹⁷⁷Lu (lutetium-177) or halide thereof such as ¹⁷⁷LuCl₃ with EuK-Sub-kf-iodo-y-DOTAGA (structures 1A and/or 1B above) as disclosed in WO2022/013610.

The above structures 1A and/or 1B may be suitably formed as described previously such as in Weineisen et al. J Nucl Med 2015; 56:1169-1176; and Chatalic, Theranostics, 6(6), 849-861 (2016). To provide an optically enriched or substantially optically enriched or enantiomerically pure sample of ¹⁷⁷Lu-PSMA I & T the corresponding optical isomer of EuK-Sub-kf-iodo-y-DOTAGA may be used in the incorporation reaction. That is, the compound of structure 1A may be reacted with lutetium-177 to provide the R isomer complex of structure 2A-2, 2A-3, and 2A-4, and the compound of structure 1B may be reacted with lutetium-177 to provide the S isomer complex of structure 2B-1, 2B-2, 2B-3, and 2B-4. The structures 1A and/or 1B also are available from piCHEM (RaabaGrambach, Austria). Optically enriched mixtures of structures 1A and/or 1B suitably may be prepared with use of an optically enriched precursor (reagent) and/or separation of optical isomers with an appropriate optically active reagent such as an optically active salt.

It is understood that ¹⁷⁷Lu-PSMA I & T as referred to herein includes the above structures 2A and/or 2B as well as other complexes of lutetium (¹⁷⁷Lu) and EuK-Sub-kf-iodo-y-DOTAGA. For instance, references herein to ¹⁷⁷Lu-PSMA I & T include compounds that generally correspond to structure 2A-2, 2A-3, 2A-4, 2B-1, 2B-2, 2B-3, and/or 2B-4 but where the ¹⁷⁷Lu substantially complexes to other portions or moieties (such as one or more other nitrogens) of the EuK-Sub-kf-iodo-y-DOTAGA molecule than as depicted in structures 2A and 2B above. References to ¹⁷⁷Lu-PSMA I & T also may include other stereoisomers than those shown in structures 1A, 1B, 2A-2, 2A-3, 2A-4, 2B-1, 2B-2, 2B-3, and 2B-4 above, although the stereoisomers depicted in structures 1A, 1B, 2A-1, 2A-2, 2A-3, 2A-4, 2B-1, 2B-2, 2B-3, and 2B-4 are preferred, particularly structures 1A and 2A.

To synthesize ¹⁷⁷Lu-PSMA I & T, lutetium-177 (¹⁷⁷Lu) can be admixed with EuK-Sub-kf-iodo-y-DOTAGA. The ¹⁷⁷Lu suitably may be carrier added or more preferably no-carrier-added (n.c.a.) lutetium-177. To facilitate incorporation (e.g. complexing including chelating) of lutetium-177 with the EuK-Sub-kf-iodo-y-DOTAGA compound, preferably an admixture of the compounds is thermally treated.

As discussed, it has been found that substantially complete incorporation of lutetium-177 and EuK-Sub-kf-iodo-y-DOTAGA can be accomplished under comparatively mild conditions including relatively short heating times such as up to or less than 30, 25, 20, 15, 12, 10, 9, 8, 7, 6 or 5 minutes and/or reduced temperatures such as up to or less than 99° C., 98° C., 97° C., 96° C., 95° C., 94° C., 93° C., 92° C., 91° C. or 90° C., or even lower temperatures for the incorporation reaction such as up to or less than 95° C., 90° C., 85° C., 80° C., 75° C., 70° C., 65° C., 60° C., 55° C., 50° C., 45° C. or 40° C. including for relatively short heating times such as up to or less than 30, 25, 20, 15, 12, 10 or 8 minutes.

In some embodiments, an admixture of lutetium-177 and EuK-Sub-kf-iodo-y-DOTAGA is heated for 20 minutes of less. In certain preferred embodiments, the admixture is heated for 15 minutes of less. In certain preferred embodiments, the admixture is heated for 12 minutes of less. In certain preferred embodiments, the admixture is heated for between about 8 and 12 minutes. For example, the admixture is heated for up to or less than about 8 minutes, for up to or less than about 9 minutes, for up to or less than about 10 minutes, for up to or less than about 11 minutes, or for up to or less than about 12 minutes. In certain preferred embodiments, the admixture is heated for at least about 8, 9, 10, 11 or 12 minutes.

In some embodiments, an admixture of lutetium-177 and EuK-Sub-kf-iodo-y-DOTAGA is heated at about 98° C. or less. In certain preferred embodiments, the admixture is heated at about 90° C.±5° C. For example, the admixture is heated at about 85° C., at about 86° C., at about 87° C., at about 88° C., at about 89° C., at about 90° C., at about 91° C., at about 92° C., at about 93° C., at about 94° C., or at about 95° C. Lower temperatures for the lutetium-177 incorporation also may be employed such as up to or less than 95° C., 90° C., 85° C., 80° C., 75° C., 70° C., 65° C., 60° C., 55° C., 50° C., 45° C. or 40° C. including as discussed above for relatively short heating times such as up to or less than 30, 25, 20, 15, 12, 10 or 8 minutes.

In some embodiments, a formulation including EuK-Sub-kf-iodo-y-DOTAGA and one or more ascorbate compounds is admixed with lutetium-177. In certain preferred embodiments, an acidic aqueous formulation of lutetium-177 is admixed with EuK-Sub-kf-iodo-y-DOTAGA and one or more ascorbate compounds (a Reaction Composition comprising one or more ascorbate compounds).

As referred to herein, an ascorbate compound suitably may include for example ascorbic acid or an ascorbate salt such as sodium L-ascorbate, among others.

In certain preferred embodiments, a hydrogen halide or acid halide aqueous formulation of lutetium-177 is admixed with EuK-Sub-kf-iodo-y-DOTAGA, including together with one more ascorbate compounds. In certain preferred embodiments, a hydrochloride acid aqueous formulation of lutetium-177 is admixed with EuK-Sub-kf-iodo-y-DOTAGA, including with one or more ascorbate compounds.

It is generally preferred that admixture of lutetium-177 and EuK-Sub-kf-iodo-y-DOTAGA is agitated during heat treatment, for example the admixture is stirred or shaken during a portion or substantially all of the heat treatment.

In some embodiments, the produced complex of lutetium-177 and EuK-Sub-kf-iodo-y-DOTAGA does not contain a gentisate adduct impurity that has a retention time of the 9 to 12 minute region, in particular aspects the 9 or 9.5 to 10.5 or 11 minute region or the about 10.2 minute region as shown in the spectra of FIGS. 1C and 1D and by HPLC analysis as defined herein, i.e. high-performance liquid chromatography with a Waters XBridge BEH Phenyl-Hexyl Column, 130 Å, 3.5 μm, 4.6 mm×150 mm using 0.1% trifluoracetic acid in water (Mobile Phase A) and 0.1% trifluoracetic acid in acetonitrile (Mobile Phase B), with a linear gradient from 85% Mobile Phase A to 55% Mobile Phase A being used over 12 minutes and the ratio was held for 15 minutes.

In some embodiments, the incorporation of lutetium-177 and EuK-Sub-kf-iodo-y-DOTAGA is greater than 98 mole percent. In certain preferred embodiments, the molar incorporation of lutetium-177 and EuK-Sub-kf-iodo-y-DOTAGA is greater than 99 mole percent, including 99.5, 99.6, 99.7, 99.8 and 99.9 mole percent.

In some embodiments, the radiochemical purity of the complex of lutetium-177 and EuK-Sub-kf-iodo-y-DOTAGA formulated as disclosed herein with one or more ascorbate compounds and optionally with one or more gentisate compounds is at least or up to 95%, 96%, 97% or 98% for 3, 4 or 5 days or more following the incorporation reaction and subsequent formulation with ascorbate compound(s) and optional gentisate compound(s) with storage of the ¹⁷⁷Lu-PSMA I & T at a temperature of 30° C. or less.

Such levels of radiochemical purity and incorporation of lutetium-177 and EuK-Sub-kf-iodo-y-DOTAGA can be provided by the reaction product of the syntheses disclosed herein and formulation of such reaction product with one or more ascorbate compounds and optionally one or more gentisate compounds without further treatment (particularly purification) step such as chromatography. Thus, significantly, the formulated lutetium-177 incorporation reaction admixture can be directly packaged (for example, stored in a sealed vial or IV bag) following formulation of the incorporation reaction product with such high purity ¹⁷⁷Lu-PSMA I & T without the need for a purification or other treatment step to remove impurities.

If desired however ¹⁷⁷Lu-PSMA I & T prepared as disclosed herein may be further treated following incorporation of lutetium-177 and EuK-Sub-kf-iodo-y-DOTAGA, for example through HPLC or other chromatography or other purification treatment.

Preferred preparations of ¹⁷⁷Lu-PSMA I & T may include one or more and preferably each of the following steps 1-6:

1. Provide lutetium-177 such as in a vial that can serve as a reaction vessel. The lutetium-177 suitably may be present in an aqueous acidic formulation, such as an HCl formulation.

2. Admix EuK-Sub-kf-iodo-y-DOTAGA with an aqueous buffer composition (Reaction Composition) that contains one or more ascorbate compound such as one or more of sodium L-ascorbate and ascorbic acid.

3. Admix the EuK-Sub-kf-iodo-y-DOTAGA composition from step 2 with the lutetium-177 formulation of step 1. For example, the EuK-Sub-kf-iodo-y-DOTAGA composition can be added to a vial that contains the lutetium-177.

4. The admixture of step 3 containing EuK-Sub-kf-iodo-y-DOTAGA and lutetium-177 then can be heated preferably with agitation, for example shaking with heating at 40-99° C. or 70-99° C., or 80-98° C. for up to 5, 6, 7, 8, 9, 10, 11, 12, 13, 14, 15, 16, 17, 18, 19 or 20 minutes. Lower heating temperatures may be preferred, such as up to 40° C., 50° C., 60° C., 70° C., 80° C., 90° C. or 95° C. for up to 6, 7, 8, 9, 10, 11, 12, 13, 14, 15, 16, 17, 18, 19 or 20 minutes.

5. At the end of the heating treatment of step 4, an aqueous composition (Formulation Composition) containing one or more gentisate compounds and preferably one or more gentisate compounds, is added to the vial or other reaction vessel. For instance, within about 0.1, 0.25, 0.5, 1, 2, 3, 4 or 5 minutes after commencing reduction or termination of heating, an aqueous composition (Formulation Composition) containing one or more gentisic compounds is added to the vial or other reaction vessel. Reduction or termination of heating can include physical removal of the heating source from the reaction vessel, or termination of power to the heating element.

6. The admixture of step 5 then may be transferred to a vessel containing an aqueous composition that comprises one or more gentisate compounds and preferably one or more ascorbate compounds. The mixture may be filtered and transferred to a container such as a syringe, vial or IV bag. Desired dosages can be dispensed for administration to a patient preferably within 5, 4, or 3 days from completing step 5 above.

Additional radiopharmaceutical compounds can be prepared by known methods, including as disclosed herein.

### Pharmaceutical Compositions

In certain preferred embodiments, the pharmaceutical composition is free of unchelated radionuclide such as lutetium-177 or ⁶⁷Cu, ²²⁵Ac, ²¹¹At and/or ¹³¹I in an amount of not more than 2, 1.5, 1.0 or 0.5 weight % based on total weight of the pharmaceutical composition, such as may be determined by radiometric detection (including HPLC radiometric detection), where the composition is maintained at 30° C. or less and such purity levels are exhibited for 3, 4 or 5 days or more following preparation of the composition.

In additional preferred embodiments, a pharmaceutical composition is free of radiochemical impurities in an amount of not more than 5, 4, 3.5, 3, 2.5, 2, 1.5, 1 or 0.5 weight % based on total weight of the pharmaceutical composition, such as may be determined by radiometric detection (including HPLC radiometric detection), where the composition is maintained at 30° C. or less and such purity levels are exhibited for 3, 4 or 5 days or more following preparation of the composition.

In yet still additional preferred embodiments, a pharmaceutical composition is free of chemical impurities in an amount of not more than 5, 4, 3, 2, 1 or 0.5 weight % based on total weight of the pharmaceutical composition, such as may be determined by chromatography or other method including HPLC or HPLC/UV analysis, where the composition is maintained at 30° C. or less and such purity levels are exhibited for 3, 4 or 5 days or more following preparation of the composition.

In yet still additional preferred embodiments, the pharmaceutical composition is 1) free of unchelated radionuclide such as of ¹⁷⁷Lu, ⁶⁷Cu, ²²⁵Ac, ²¹¹At and/or ¹³¹I in an amount of not more than 2, 1.5, 1.0 or 0.5 weight % (such as may be determined by radiometric detection (including HPLC radiometric detection)); 2) free of radiochemical impurities in an amount of not more than 5, 4, 3.5, 3, 2.5, 2, 1.5, 1 or 0.5 weight % (such as may be determined by radiometric detection (including HPLC radiometric detection); and 3) free of chemical impurities in an amount of not more than 5, 4, 3, 2, 1 or 0.5 weight % (such as may be determined by HPLC/UV analysis), with all weight % based on total weight of the pharmaceutical composition, and where the composition is maintained at 30° C. or less and such purity levels are exhibited for 3, 4 or 5 days or more following preparation of the composition.

In certain embodiments, the pharmaceutical composition is formulated for parenteral administration, such as intravenous, intramuscular, intradermal, subcutaneous, intrathecal or intraperitoneal administration. For example, the pharmaceutical composition is formulated for intravenous, intramuscular, subcutaneous or intradermal injection. In preferred aspects, the pharmaceutical composition is formulated for intravenous administration. In typical embodiments, the pharmaceutical composition may be administered in a form of a pharmaceutical aqueous solution.

In certain embodiments, the pharmaceutical composition is an aqueous solution, dispersion or other admixture such as for injection and comprises the radiopharmaceutical compound (such as a radiopharmaceutical compound of any one of Formulae X and/or I through VI and/or O above, or any one of Compounds A through N and a radioisotope such as Lutetium-177, Copper-67, Lead-212, Terbium-161 and/or Actinium-225 and one or more gentisate compounds and preferably one or more ascorbate compounds. In further preferred embodiments, the pharmaceutical composition is an aqueous solution, dispersion or other admixture such as for injection and comprises 1) a radiopharmaceutical compound (such as a radiopharmaceutical compound of any one of Formulae X and/or I through VI and/or O above, or any one of Compounds A through N and a radioisotope such as Lutetium-177, Copper-67, Lead-212, Terbium-161, and/or Actinium-225 and 2) one or more gentisate compounds and preferably one or more ascorbate compound.

In certain preferred embodiments, a pharmaceutical aqueous solution, dispersion or admixture is provided where the radiopharmaceutical compound (such as a radiopharmaceutical compound of any one of any one of Formulac X and/or I through VI and/or O above, or any one of Compounds A through N and a radioisotope such as Lutetium-177, Copper-67, Lead-212, Terbium-161, and/or Actinium-225 is suitably present in a concentration that it provides a volumetric radioactivity of at least 100 MBq/mL, preferably of at least 250, 500, 750 or 1000 MBq/mL within 1, 2, 3, 4 or 5 days following preparation. In certain aspects, a radiopharmaceutical compound (such as a radiopharmaceutical compound of any one of Formulae X and/or I through VI and/or O above, or any one of Compounds A through N and a radioisotope such as Lutetium-177, Copper-67, Lead-212, Terbium-161, and/or Actinium-225 is present in a concentration that it provides a volumetric radioactivity of from 100 to 1000 MBq/mL, preferably from or up to about 250, 500, 750 or 1000 MBq/mL within 1, 2, 3, 4 or 5 days following preparation.

In certain aspects, the one or more stabilizer compounds (i.e. a gentisate compound and preferably one or more ascorbate compounds) may be present in a total concentration of at least 5 mg/mL, preferably at least 10 mg/mL of an aqueous pharmaceutical composition.

In certain aspects, the one or more stabilizer compounds are one or more of gentisic acid (2,5-dihydroxybenzoic acid) or salts thereof, ascorbic acid (L-ascorbic acid) or salts thereof (e.g. sodium ascorbate), methionine, histidine, melatonin, N-acetylmethionine, ethanol, or Se-methionine, preferably ascorbic acid or salts thereof and gentisic acid or salts thereof.

In certain aspects, the pharmaceutical aqueous formulation has a shelf life of at least 24 hours at about 30° C. or less, at least 48 hours at about 30° C. or less, at least 72 hours at 30° C. or less, or from 24 hours to 120 hours at 30° C. or less, from 24 hours to 96 hours at 30° C. or less, from 24 hours to 84 hours at 30° C. or less, from 24 hours to 72 hours at 30° C. or less, in particular a shelf life of at least 72 hours at 30° C. or less. In further particular aspects, the pharmaceutical aqueous formulation has a shelf life of at least 96 hours (or 4 days) at about 30° C. or less, or the pharmaceutical aqueous formulation has a shelf life of at least 120 hours (or 5 days) at about 30° C. or less, or the pharmaceutical aqueous formulation has a shelf life of at least 144 hours (or 6 days) at about 30° C. or less.

In certain aspects, one, two or three total distinct stabilizer compounds are present during the formation of the radiopharmaceutical composition (such as a radiopharmaceutical compound of any one of Formulae X and/or I through VI and/or O above, or any one of Compounds A through N and a radioisotope such as Lutetium-177, Copper-67, Lead-212, Terbium-161, and/or Actinium-225 and ²²⁵Ac-PSMA I & T, ⁶⁷Cu-PSMA I & T and/or ¹⁷⁷Lu-PSMA I & T, preferably in an amount to result in a concentration of from 5 mg/mL or more of the 1-3 stabilizer compounds. As discussed, preferably at least one of the stabilizer compounds will be an ascorbate compound.

In certain embodiments, a pharmaceutical aqueous solution may further include a sequestering agent, for example added after formation of a radiopharmaceutical compound (such as a radiopharmaceutical compound of any one of any one of Formulae X and/or I through VI and/or O above, or any one of Compounds A through N and a radioisotope such as Lutetium-177, Copper-67, Lead-212, Terbium-161, and/or Actinium-225 Formulae X and/or I through VI above, or any one of Compounds A through N and ²²⁵Ac-PSMA I & T, ⁶⁷Cu-PSMA I & T and/or ¹⁷⁷Lu-PSMA I & T, suitably to remove uncomplexed radionuclide. Suitable sequestering agents may include for example diethylenetriaminepentaacetic acid (DTPA) or a salt thereof, suitably in an amount to result in a concentration of from 0.01 to 0.50 mg/mL of the aqueous pharmaceutical composition such as a compound comprising one or more of a radiopharmaceutical compound of any one of Formulae X and/or I through VI and/or O above, or any one of Compounds A through N and a radioisotope such as Lutetium-177, Copper-67, Lead-212, Terbium-161, and/or Actinium-225.

In a particularly preferred aspect, a pharmaceutical composition (such as a composition comprising compound of any one of Formulae X and/or I through VI and/or O above, or any one of Compounds A through N and a radioisotope such as Lutetium-177, Copper-67, Lead-212, Terbium-161, and/or Actinium-225 and 25Ac-PSMA I & T, ⁶⁷Cu-PSMA I & T and/or ¹⁷⁷Lu-PSMA I & T is provided as a sterile solution for intravenous use. In one embodiment, a single-dose vial suitably will contain 6.8+/−10% GBq ¹⁷⁷Lu-PSMA I & T for example calibrated at 1, 2, 3, 4, 5 or 6 or more days post-day of manufacture in 10 to 14 mL formulated with one or more radioprotectants and may include a buffer. The pH range of the solution is preferably 5.0 to 7.0. As discussed, radioprotectants or stabilizers may be one or more gentisate compounds and optionally together with one or more ascorbate compounds.

The pharmaceutical composition (such as a composition comprising a compound of any one of Formulae X and/or I through VI and/or O above, or any one of Compounds A through N and a radioisotope such as Lutetium-177, Copper-67, Lead-212, Terbium-161, and/or Actinium-225 and ²²⁵Ac-PSMA I & T, ⁶⁷Cu-PSMA I & T and/or ¹⁷⁷Lu-PSMA I & T I & T also may be provided in a multi-dose format or packaging, such as a multi-dose vial that contains multiple doses, such as 2, 3, 4, 5, 6, 7, 8, 9, or 10 or more doses of the pharmaceutical composition.

### Methods of Treatment

As discussed, compounds and compositions disclosed herein (such as a composition comprising compound of any one of Formulae X and/or I through VI and/or O above, or any one of Compounds A through N and a radioisotope such as Lutetium-177, Copper-67, Lead-212, Terbium-161, and/or Actinium-225 and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T are provided to treat cancers, including prostate cancer, for example non-metastatic prostate cancer and metastatic prostate cancer, including hormone sensitive prostate cancer, castration resistant prostate cancer (CRPC) and drug-resistant prostate cancer, such as anti-androgen drug (e.g., enzalutamide) resistant prostate cancer.

In such methods, compounds and compositions disclosed herein (such as a composition comprising compound of any one of Formulae X and/or I through VI and/or O above, or any one of Compounds A through N and a radioisotope such as Lutetium-177, Copper-67, Lead-212, Terbium-161, and/or Actinium-225 and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T can be administered to a subject such as a human in an amount effective to treat the cancer (e.g., reduction of tumor size), such as at a dose of about 0.1 GBq to about 30 GBq be suitably administered from a unit dose in a vial or a syringe or as a bulk solution in a vial or a syringe prepared from a cold-kit prepared with lutetium-177 at a local or central nuclear pharmacy or through cGMP central manufacturing.

In certain embodiments, the subject is suffering from prostate cancer such as one or more of castration-sensitive prostate cancer, castration-resistant prostate cancer, metastatic castration-resistant prostate cancer, advanced stage prostate cancer, drug-resistant prostate cancer such as anti-androgen-resistant prostate cancer (e.g., enzalutamide-resistant prostate cancer, abiraterone-resistant prostate cancer, bicalutamide-resistant prostate cancer), docetaxel-resistant prostate cancer, PARP resistant prostate cancer, radium chloride resistant prostate cancer, AR-V7-induced drug-resistant prostate cancer such as AR-V7-induced enzalutamide-resistant prostate cancer, AKR1C₃-induced drug-resistant prostate cancer such as AKR1C3-induced enzalutamide-resistant prostate cancer, and combinations thereof.

In particular embodiments, the subject is a human suffering metastatic castration-resistant prostate cancer and an effective amount of a compound or composition disclosed herein (such as a composition comprising compound of any one of Formulae X and/or I through VI and/or O above, or any one of Compounds A through N and a radioisotope such as Lutetium-177, Copper-67, Lead-212, Terbium-161, and/or Actinium-225 and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T is administered to the subject to treat the prostate cancer.

In additional particular embodiments, the subject is a human suffering oligometastatic hormone sensitive prostate cancer and an effective amount of a compound or composition disclosed herein (such as a composition comprising compound of any one of Formulae X and/or I through VI and/or O above, or any one of Compounds A through N and a radioisotope such as Lutetium-177, Copper-67, Lead-212, Terbium-161, and/or Actinium-225 and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T is administered to the subject to treat the prostate cancer.

In further particular embodiments, the subject is a human suffering metastatic castration-resistant prostate cancer and an effective amount of a compound or composition disclosed herein (such as a composition comprising compound of any one of Formulae X and/or I through VI and/or O above, or any one of Compounds A through N and a radioisotope such as Lutetium-177, Copper-67, Lead-212, Terbium-161, and/or Actinium-225, or any one or more of Compounds A through N and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T is administered to the subject to treat the prostate cancer.

The effective amount of a radiopharmaceutical compound or composition disclosed herein (such as a composition comprising compound of any one of Formulae X and/or I through VI and/or O above, or any one of Compounds A through N and a radioisotope such as Lutetium-177, Copper-67, Lead-212, Terbium-161, and/or Actinium-225 and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T administered to a patient will generally be determined by considering the patient record. However, in certain aspects, the effective amount suitably may be within a range of about 0.1 GBq to 30 GBq per dose. More specifically, the dose may range from about 1 GBq to about 20 GBq or about 30 GBq per dose subject, for example, about 1, 1.5, 2, 2.5, 3, 3.5, 4, 4.5, 5, 5.5, 6, 6.5, 6.8, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 21.5, 22, 22.5, 23, 23.5, 24, 24.5, 25, 25.5, 26, 26.5, 27, 27.5, 28, 28.5, 29, 29.5 or 30 GBq per dose of a radiopharmaceutical compound or composition disclosed herein (such as a composition comprising compound of any one of Formulae X and/or I through VI and/or O above, or any one of Compounds A through N and a radioisotope such as Lutetium-177, Copper-67, Lead-212, Terbium-161, and/or Actinium-225 and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T, or any range between two of the above values. The dose can be administered from a unit dose in a vial or a syringe or as a bulk solution in a vial or a syringe prepared from a cold-kit prepared with a radioisotope such as ¹⁷⁷Lu, ²²⁵Ac, ⁶⁷Cu, at a local or central nuclear pharmacy or through cGMP central manufacturing.

If necessary or desirable, the treatment may involve more than one administration of an effective amount of a radiopharmaceutical compound or composition disclosed herein (such as a composition comprising one or more compounds of any one of Formulae X and/or I through VI and/or O above, or any one of Compounds A through N and a radioisotope such as Lutetium-177, Copper-67, Lead-212, Terbium-161, and/or Actinium-225 and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T. It can be generally beneficial to repeat the administration of a radiopharmaceutical compound or composition disclosed herein (such as a composition comprising one or more compounds of any one of Formulae X and/or I through VI above, or any one or more of Compounds A through N and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T to the subject after 7 to 56 days, such as at a 4 to 8 week interval.

In a particularly preferred protocol, a radiopharmaceutical compound or composition disclosed herein (such as a composition comprising one or more compounds of any one of any one of Formulae X and/or I through VI and/or O above, or any one of Compounds A through N and a radioisotope such as Lutetium-177, Copper-67, Lead-212, Terbium-161, and/or Actinium-225 and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T, dosage form is a sterile aqueous solution that is administered by intravenous injection. The dosing regimen may include multiple infusions such as 1, 2, 3, 4, 5, 6, 7, 8, 9 or 10 infusions at effective dosages such as of 6.8 GBq+/−10% each, administered about 1, 2, 3, 4, 5, 6, 7, 8, 9 or 10 weeks apart.

### Combination Therapy

A radiopharmaceutical compound or composition disclosed herein (such as a composition comprising one or more compounds of any one of Formulae X and/or I through VI and/or O above, or any one of Compounds A through N and a radioisotope such as Lutetium-177, Copper-67, Lead-212, Terbium-161, and/or Actinium-225 and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T and/or ¹⁷⁷Lu-PSMA I & T suitably may be administered to a subject in conjunction or combination with one or more other therapeutic agents, particularly one or more other chemotherapeutic agents.

In one aspect, a subject may receive treatment with a radiopharmaceutical compound or composition disclosed herein (such as a composition comprising one or more compounds of any one of Formulae I through VI above, or any one or more of Compounds A through N and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T in combination with a regime of docetaxel and/or prednisone, particularly for a subject suffering from castration resistant prostate cancer.

In another aspect, a subject may receive treatment with a radiopharmaceutical compound or composition disclosed herein (such as a composition comprising one or more compounds of any one of Formulae X and/or I through VI and/or O above, or any one of Compounds A through N and a radioisotope such as Lutetium-177, Copper-67, Lead-212, Terbium-161, and/or Actinium-225 and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T in combination with a regime that can include chemotherapy such as docetaxel; cisplatin; gemcitabine; cisplatin/gemcitabine; cabazitaxel; one or more antiandrogens such as one or more LHRH agonists, such as leuprolide and goserelin, or antagonists (e.g. firmagon and relugolyx); one or more antiandrogens such as flutamide, nilutamide, bicalutamide, cyproterone, abiraterone, enzalutamide, darolutamide and apalutamide; one or more PARP inhibitors such as olaparib, rucaparib or niraparib, particularly for a subject suffering from prostate cancer including metastatic castration resistant prostate cancer.

In additional aspects, a subject may receive treatment with a radiopharmaceutical compound or composition disclosed herein (such as a composition comprising one or more compounds of any one of Formulae X and/or I through VI and/or O above, or any one of Compounds A through N and a radioisotope such as Lutetium-177, Copper-67, Lead-212, Terbium-161, and/or Actinium-225 and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T in combination with an immunotherapy regime which may include adoptive cell therapies or adoptive immunotherapy.

For example, to treat a patient suffering from cancer, a radiopharmaceutical compound or composition disclosed herein (such as a composition comprising one or more compounds of any one of Formulae X and/or I through VI and/or O above, or any one of Compounds A through N and a radioisotope such as Lutetium-177, Copper-67, Lead-212, Terbium-161, and/or Actinium-225 and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T may be administered in combination with immune effector cells (e.g., T cells, NK cells) engineered to express a Chimeric Antigen Receptor (e.g. CAR T-cell therapy), including to treat a cancer or a disease associated with expression of a tumor antigen.

For a patient suffering from cancer including prostate cancer, a radiopharmaceutical compound or composition disclosed herein (such as a composition comprising one or more compounds of any one of Formulae X and/or I through VI and/or O above, or any one of Compounds A through N and a radioisotope such as Lutetium-177, Copper-67, Lead-212, Terbium-161, and/or Actinium-225 and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T also may be administered in combination with other immune-based therapies such as sipuleucel-T (Provenge) or other immune-boosting approaches including antibody treatments. For instance, in one protocol, a radiopharmaceutical compound or composition disclosed herein (such as a composition comprising one or more compounds of any one of Formulae X and/or I through VI and/or O above, or any one of Compounds A through N and a radioisotope such as Lutetium-177, Copper-67, Lead-212, Terbium-161, and/or Actinium-225 and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T may be administered in combination with one or more monoclonal antibodies such as pembrolizumab (Keytruda), ipilimumab (Yervoy) and/or nivolumab (Opdivo) for treating a patient suffering from cancer, particularly prostate cancer.

As used herein, the term “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject in need of treatment as disclosed herein. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

### Packaged Radiopharmaceuticals and Kits

As discussed above, treatment kits are also provided, including cold kits where a radiopharmaceutical compound or composition disclosed herein (such as a composition comprising one or more compounds of any one of Formulae X and/or I through VI and/or O above, or any one of Compounds A through N and a radioisotope such as Lutetium-177, Copper-67, Lead-212, Terbium-161, and/or Actinium-225 and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T can be prepared shortly before administration such as in a medical facility, for example a hospital laboratory or nuclear pharmacy. In such a kit for a radiopharmaceutical compound or composition disclosed herein (such as a composition comprising one or more compounds of any one of Formulae X and/or I through VI and/or O above, or any one of Compounds A through N and a radioisotope such as Lutetium-177, Copper-67, Lead-212, Terbium-161, and/or Actinium-225 and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T may be provided in a vial or other container in lyophilized or other form separate from a radioisotope (e.g. as ¹⁷⁷Lu, ²²⁵Ac, ⁶⁷Cu). The carrier compound and radioisotope such as EuK-Sub-kf-iodo-y-DOTAGA are reacted as disclosed herein at the medical facility to provide the radiopharmaceutical compound which then can be promptly administered to a patient.

In a further aspect, packaged preparations or products of a radiopharmaceutical compound such as a compound disclosed herein (such as a composition comprising one or more compounds of any one of Formulae X and/or I through VI and/or O above, or any one of Compounds A through N and a radioisotope such as Lutetium-177, Copper-67, Lead-212, Terbium-161, and/or Actinium-225 and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T are also provided. A packaged preparation may comprise 1) radiopharmaceutical compound optionally 2) instructions for using the radiopharmaceutical compound for treating a cancer such as prostate cancer. Preferably, the packaged preparation will comprise a therapeutically effective amount of the radiopharmaceutical compound such as a composition comprising one or more compounds of any one of Formulae X and/or I through VI and/or O above, or any one of Compounds A through N and a radioisotope such as Lutetium-177, Copper-67, Lead-212, Terbium-161, and/or Actinium-225 and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T.

In certain exemplary packaged preparations or products, a composition comprising one or more compounds of any one of Formulae X and/or I through VI and/or O above, or any one of Compounds A through N and a radioisotope such as Lutetium-177, Copper-67, Lead-212, Terbium-161, and/or Actinium-225 and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T suitably can be packaged in suitable containers labeled, for example, for use as a therapy to treat a subject suffering from cancer such as prostate cancer. The containers can include a composition comprising one or more compounds of any one of Formulae X and/or I through VI and/or O above, or any one of Compounds A through N and a radioisotope such as Lutetium-177, Copper-67, Lead-212, Terbium-161, Radium-223 and/or Actinium-225 and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T and suitably one or more ascorbate compounds and one or more gentisate compounds as disclosed herein. A product can include a container (e.g., a vial or the like) containing a composition comprising one or more compounds of any one of Formulae X and/or I through VI and/or O above, or any one of Compounds A through N and a radioisotope such as Lutetium-177, Copper-67, Lead-212, Radium-223, Terbium-161, and/or Actinium-225 and ²¹²Pb-PSMA I & T, ¹⁶¹Tb-PSMA I & T, ²²⁵Ac-PSMA I & T or ⁶⁷Cu-PSMA I & T, and/or ¹⁷⁷Lu-PSMA I & T. In addition, an article of manufacture or kit further may include, for example, packaging materials, instructions for use, syringes, delivery devices, for treating the targeted condition, such as prostate cancer or other cancer.

A packaged system or product may also include a legend (e.g., a printed label or insert or other medium (e.g., an audio or video file) describing the product's use). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents.

### EXAMPLE

The following non-limiting examples are illustrative.

### Example 1

Radiolabeling was performed using no-carrier added ¹⁷⁷LuCl₃. Radiolabeling was done either directly in the isotope vial, or transferred to a 20 mL vial. ¹⁷⁷Lu-PSMA-I & T is prepared as described in Weineisen et al. J Nucl Med 2015; 56:1169-1176 by use of racemic EuK-Sub-kf-iodo-y-DOTAGA. ¹⁷⁷Lu-PSMA I & T having an optical excess of structure 2A is also prepared as described in Weineisen et al. J Nucl Med 2015; 56:1169-1176 using the R-isomer of EuK-Sub-kf-iodo-y-DOTAGA (i.e. the compound of structure 1A above). The racemic ¹⁷⁷Lu-PSMA-I & T or ¹⁷⁷Lu-PSMA I & T having an optical excess of structure 2A was diluted using reaction buffer and was added to a reaction vial to form a Reaction Composition. A thus prepared Reaction Composition was heated with mixing using a shaker at 90+/−4° C. Different reaction times and cool down periods were tested during development, ranging from 10 to 30 minutes of labelling time and 0 to 20 minutes of cool down time. Three different Reaction Compositions ((A), (B) and (C) set forth below) were assessed.

The formulated racemic ¹⁷⁷Lu-PSMA-I & T and ¹⁷⁷Lu-PSMA I & T having an optical excess of structure 2A batches were sampled for quality control testing and tested for stability for up to 6 days post-day of manufacture. Batches of both 1) racemic ¹⁷⁷Lu-PSMA-I & T and 2) ¹⁷⁷Lu-PSMA I & T having an optical excess of structure 2A at room temperature (25° C.) and 2-8° C. storage were evaluated during development. Reverse-phase HPLC with radiometric and UV detection and thin layer chromatography with radiometric detection were used to assess chemical and radiochemical impurities.
(1) Reaction Buffer Compositions evaluated:
   (A) 99 to 247 mg/mL Ascorbate (pH 4.5 to 6), made from sodium L-ascorbate and ascorbic acid (preferred condition 162 mg/mL, pH 5.5 to 6.0)
   (B) 5.1 to 12.5 mg/mL Gentisic Acid (0.4 M acetate used as buffer, pH 5-6.5), made from gentisic acid
   (C) 100 mg/mL Ascorbate with 5.0 mg/mL Gentisic Acid (pH 5-6.5), made from sodium L-ascorbate, ascorbic acid and gentisic acid
   (D) Reaction Buffer Compositions were diluted 1.48:1-0.4N HCl:Reaction buffer
(2) Formulation Buffer Compositions evaluated:
   (A) 65 to 68 mg/mL Ascorbate with 25 to 28 mg/mL Gentisic Acid (pH 6-7), made from sodium L-ascorbate and gentisic acid (preferred condition)
   (B) 64 to 86 mg/mL Ascorbate (pH 6-7), made from sodium L-ascorbate
   (C) Formulation Buffer Compositions were diluted 11.5:1-Formulation Buffer:Reaction Composition

The conditions as set forth in the following Table 7 were evaluated:

TABLE 7

| | | | | | | | Radioactivity |
|---|---|---|---|---|---|---|---|
| | Reaction Compositions | | | | Formulation | | Concentration |
| | | | | Reaction | Compositions | | at Activity |
| | Gentisic | | Acetate | Time | Gentisic | | Reference |
| | Acid | Ascorbate | (buffer) | (Min) | Acid | Ascorbate | Time |
| Experiment | + | − | + | 30 | − | + | 0.93 |
| 1 | | | | | | | |
| Experiment | + | − | + | 30 | − | + | 0.97 |
| 2 | | | | | | | |
| Experiment | + | − | + | 20 | − | + | 0.53 |
| 3a | | | | | | | |
| Experiment | + | − | + | 20 | + | + | 0.48 |
| 3b | | | | | | | |
| Experiment | + | + | − | 20 | − | + | 0.58 |
| 3c | | | | | | | |
| Experiment | + | + | − | 20 | + | + | 0.56 |
| 3d | | | | | | | |
| Experiment | − | + | − | 20 | + | + | 0.49 |
| 4a | | | | | | | |
| Experiment | − | + | − | 20 | + | + | 0.49 |
| 4b | | | | | | | |
| Experiment | + | + | − | 20 | + | + | 0.49 |
| 4c | | | | | | | |
| Experiment | + | + | − | 20 | + | + | 0.49 |
| 4d | | | | | | | |
| Experiment | + | − | + | 20 | + | + | 0.49 |
| 5a | | | | | | | |
| Experiment | − | + | − | 20 | + | + | 0.49 |
| 5b | | | | | | | |
| Experiment | + | − | + | 20 | + | + | 0.49 |
| 6a | | | | | | | |
| Experiment | − | + | − | 20 | + | + | 0.49 |
| 6b | | | | | | | |
| Experiment | − | + | − | 20 | + | + | 0.49 |
| 7 | | | | | | | |
| Experiment | − | + | − | 15 | + | + | 0.49 |
| 8 | | | | | | | |
| Experiment | − | + | − | 10 | + | + | 0.49 |
| 9 | | | | | | | |
| Experiment | − | + | − | 10 | + | + | 0.49 |
| 10 | | | | | | | |
| Experiment | − | + | − | 10 | + | + | 0.49 |
| 11 | | | | | | | |
| Experiment | − | + | − | 10 | + | + | 0.49 |
| 12 | | | | | | | |

In each of Examples 1, 2, 3a, 3b, 3c, 3d, 4a, 4b, 4c and 4d, racemic EuK-Sub-kf-iodo-y-DOTAGA and racemic ¹⁷⁷Lu-PSMA I & T was used. In each of Examples 5a, 5b, 6a, 6b, 7, 8, 9, 10, 11 and 12 R-isomer enriched EuK-Sub-kf-iodo-y-DOTAGA (1A above) and ¹⁷⁷Lu-PSMA I & T having an optical excess of structure 2A were used.

### Results:

### 1. Lu-177 Incorporation:

Chromatography shows the amount of Lu-177 incorporated into the EuK-Sub-kf-iodo-y-DOTAGA was >99.5% for all reactions at completion of production. This demonstrates that under all conditions, including a reaction time limited to 10 minutes, full incorporation of Lu-177 into EuK-Sub-kf-iodo-y-DOTAGA was achieved. The ability to incorporate Lu-177 in a reduced time, reduced the number of impurities at the end of production.

### 2. Negative Impact of Gentisic Acid in the Reaction Composition (Incorporation Reaction):

In each of Experiments 1 and 2, gentisic acid was present in the Reaction Composition, but the amount of gentisic acid present in the Reaction Composition was double in Example 2 relative to Example 1. Specifically, in Experiment 1, 2.5 mg/mL of gentisic acid was used in the Reaction Composition; and in Experiment 2, 5.0 mg/mL of gentisic acid was used in the Reaction Composition. Over the 5 or 6 day evaluation period, it was found that radiochemical purity dropped 5% for the Experiment 1 sample and radiochemical purity dropped 13% for the Experiment 2 sample.

Experiments 3 through 6 demonstrated that the inclusion of only gentisic acid in the reaction results in an impurity peak that elutes at ˜10.2 min; this peak is absent when only ascorbate is used. When gentisic acid and ascorbate are both used in the reaction, the peak at ˜10.2 min also may be present (see FIGS. 1A-1D) in certain instances. Therefore, to minimize impurities, gentisic acid may be excluded from the incorporation reaction.

### 3. HPLC Verification

In FIG. 1A, HPLC shows an incorporation reaction with acetate and gentisic acid (peak at 10.2 min always appears): 20 min reactions at 90° C. In FIGS. 1B-1C, HPLC shows reactions with gentisic acid and ascorbate (peak at 10.2 min appears sometimes): 20 min reactions at 90° C. In FIG. 1D, HPLC shows reaction with just ascorbate (peak at ˜10.2 min never appears): 20 min reactions at 90° C.

### 4. Positive Impact of Gentisic Acid in the Formulation Composition

It was found that inclusion of gentisic acid in the Formulation Composition at the time of or following termination of the reaction incorporating Lu-177 with EuK-Sub-kf-iodo-y-DOTAGA favorably impacted radiochemical purity of the racemic ¹⁷⁷Lu-PSMA-I & T or ¹⁷⁷Lu-PSMA I & T having an optical excess of structure 2A. That is, inclusion of gentisic acid in the Formulation Composition showed a reduced drop in radiochemical purity over time. Thus, over the 5 or 6 day evaluation period, in Experiment 3c where gentisic acid was not present in the Formulation Composition, radiochemical purity dropped 10.2%, whereas in Experiment 3d where gentisic acid was present in the Formulation Composition, radiochemical purity dropped 7.6%.

### Example 2

Further evaluations were conducted as follows. In Table 8, Conditions 1-8 that are not reported in Example 1 are indicated by bold text. Experiments 1, 2, 3a-3d, 4a-4d, 5a-5b and 6a,6b in Table 8 below that are also disclosed in Example 1 above are without bold text in Table 8 below.

Radiolabeling was performed using no-carrier added ¹⁷⁷LuCl₃. Radiolabeling was done either directly in the isotope vial, or transferred to a 20 mL vial. ¹⁷⁷Lu-PSMA-I & T is prepared as described herein and in Weineisen et al. J Nucl Med 2015; 56:1169-117 by use of racemic EuK-Sub-kf-iodo-y-DOTAGA. ¹⁷⁷Lu-PSMA I & T having an optical excess of structure 2A is also prepared as described in Weineisen et al. J Nucl Med 2015; 56:1169-1176 using the R-isomer of EuK-Sub-kf-iodo-y-DOTAGA (i.e. the compound of structure 1A above). ¹⁷⁷Lu-PSMA-I & T is also commercially available. The racemic ¹⁷⁷Lu-PSMA-I & T or ¹⁷⁷Lu-PSMA I & T having an optical excess of structure 2A was diluted using reaction buffer and was added to a reaction vial to form a Reaction Composition. A thus prepared Reaction Composition was heated as per the method followed. Different reaction times were tested during development, ranging from 10 to 30 mins of labelling time. Three different Reaction Compositions ((A), (B) and (C) set forth below) were assessed.

The formulated racemic ¹⁷⁷Lu-PSMA-I & T and ¹⁷⁷Lu-PSMA I & T having an optical excess of structure 2A batches were sampled for quality control testing and tested for stability for up to 6 days post-day of manufacture. Batches of both racemic ¹⁷⁷Lu-PSMA-I & T and ¹⁷⁷Lu-PSMA I & T having an optical excess of structure 2A at room temperature (25° C.) and 2-8° C. storage were evaluated during development. Reverse-phase HPLC with radiometric and UV detection and thin layer chromatography with radiometric detection were used to assess chemical and radiochemical impurities.
(3) Reaction Compositions evaluated at precursor amounts of 125 to 250 μg:
   (A) 40 to 100 mg/mL Ascorbate (pH 4.5 to 6), made from sodium L-ascorbate and ascorbic acid (preferred condition 65 mg/mL, pH 5.5 to 6.0)
   (B) 2.0 to 9.8 mg/mL Gentisic Acid (27 to 35 mg/mL acetate used as buffer, pH 5-6.5), made from Gentisic acid (pH 5-6.5)
   (C) 40 mg/mL Ascorbate with 2.0 to 2.1 mg/mL Gentisic Acid, made from sodium L-ascorbate, ascorbic acid and gentisic acid
(4) Formulation Compositions evaluated:
   (A) 1 mg/mL Gentisic Acid (27 to 35 mg/mL acetate used as a buffer, pH 5.5), made from Gentisic acid
   (B) 59 to 79 mg/mL Ascorbate with 0.16 to 26 mg/mL Gentisic Acid (pH 6-7), made from sodium L-ascorbate and gentisic acid (preferred condition)

The conditions as set forth in the following Table 8 were evaluated:

TABLE 8

| | | | | | Formulation | | | |
|---|---|---|---|---|---|---|---|---|
| | Reaction Compositions | | | | Compositions (with | | | |
| | | | | Reaction | ¹⁷⁷Lu-PSMA-I&T) | | | |
| | Gentisic | | | Time | Gentisic | Sodium | | |
| | Acid | Ascorbate | | (Min)/Temp | Acid | Ascorbate | | |
| Reference | (mg/mL) | (mg/mL) | Buffer | (° C. | (mg/mL) | (mg/mL) | Isomer | RAC² |
| Condition 1 | 0 | 0 | MES¹ | 20/95 | 0 | 0 | R-isomer | 0.81 |
| Condition 2 | 3.3 | 0 | Sodium | 30/90 | 1 | 0 | R-isomer | 0.74 |
| | | | Acetate | | | | | |
| Condition 3 | 0 | 0 | Sodium | 30/90 | 1 | 0 | R-isomer | 0.74 |
| | | | Acetate | | | | | |
| Condition 4 | 9.8 | 0 | Sodium | 20/90 | 25 | 58 | R-isomer | 0.79 |
| | | | Acetate | | | | | |
| Condition 5 | 0 | 65 | none | 10/90 | 26 | 66 | R-isomer | |
| Condition 6 | 0 | 80 | none | 10/80 | 26 | 66 | R-isomer | 0.47 |
| Condition 7 | 0 | 80 | none | 10/70 | 26 | 66 | R-isomer | 0.47 |
| Condition 8 | 0 | 80 | none | 10/60 | 26 | 66 | R-isomer | 0.47 |
| Experiment 1 | 2.5 | 0 | Sodium | 30/90 | 0.20 | 59 | racemic | 0.93 |
| | | | Acetate | | | | | |
| Experiment 2 | 5.0 | 0 | Sodium | 30/90 | 0.40 | 68 | racemic | 0.97 |
| | | | Acetate | | | | | |
| Experiment | 2.1 | 0 | Sodium | 20/90 | 0.16 | 79 | racemic | 0.53 |
| 3a | | | Acetate | | | | | |
| Experiment | 2.1 | 0 | Sodium | 20/90 | 24 | 59 | racemic | 0.48 |
| 3b | | | Acetate | | | | | |
| Experiment | 2.1 | 40 | none | 20/90 | 0.16 | 82 | racemic | 0.58 |
| 3c | | | | | | | | |
| Experiment | 2.1 | 40 | none | 20/90 | 24 | 62 | racemic | 0.56 |
| 3d | | | | | | | | |
| Experiment | 0 | 40 | none | 20/90 | 23 | 62 | racemic | 0.49 |
| 4a | | | | | | | | |
| Experiment | 0 | 40 | none | 20/90 | 26 | 62 | racemic | 0.49 |
| 4b | | | | | | | | [½ |
| | | | | | | | | RAC] |
| Experiment | 2.0 | 40 | none | 20/90 | 23 | 62 | racemic | 0.49 |
| 4c | | | | | | | | [½ |
| | | | | | | | | RAC] |
| Experiment | 2.0 | 40 | none | 20/90 | 26 | 62 | racemic | 0.49 |
| 4d | | | | | | | | |
| Experiment | 3.3 | 0 | Sodium | 20/90 | 23 | 62 | R-isomer | 0.49 |
| 5a | | | Acetate | | | | | |
| Experiment | 0 | 65 | none | 20/90 | 23 | 64 | R-isomer | 0.49 |
| 5b | | | | | | | | |
| Experiment | 0 | 0 | none | 20/90 | 23 | 62 | R-isomer | 0.49 |
| 6a | | | | | | | | |
| Experiment | 0 | 100 | none | 20/90 | 23 | 63 | R-isomer | 0.49 |
| 6b | | | | | | | | |
| Experiment 7 | 0 | 65 | none | 20/90 | 23 | 63 | R-isomer | 0.49 |
| Experiment 8 | 0 | 65 | none | 15/90 | 26 | 66 | R-isomer | 0.49 |
| Experiment 9 | 0 | 65 | none | 10/90 | 26 | 66 | R-isomer | 0.49 |

¹MES = 2-(N-morpholino)ethanesulfonic acid
²RAC = Radioactivity Concentration at Activity Reference Time

### Results:

### 1. Incorporation Reaction and Formulation Compositions for Condition 1, 2 and 3 in Table 8

Radiolabeling was performed using no-carrier added ¹⁷⁷LuCl₃ following the radiolabeling conditions described by Weineisen et al. J Nucl Med 2015; 56:1169-1176 and Chatalic et al. Theranostics 2016; 6:849-861 (Chatalic) for the preparation of ¹⁷⁷Lu-PSMA-I & T for pre-clinical or clinical use. Thin layer chromatography shows the amount of Lu-177 incorporated into the EuK-Sub-kf-iodo-y-DOTAGA was >99.5% for all reactions at completion of production at reaction temperatures of 95° C. and 90° C. for 20 and 30 minutes. The conditions are described in Table 8 above.

Batches of both racemic ¹⁷⁷Lu-PSMA-I & T and ¹⁷⁷Lu-PSMA I & T having an optical excess of structure 2A at room temperature (25° C.) and 2-8° C. storage were evaluated during development. Reverse-phase HPLC with radiometric and UV detection and thin layer chromatography with radiometric detection were used to assess chemical and radiochemical impurities.

HPLC showed that radiochemical purity was 96.7% for Condition 2, 87.4% for Condition 3 and 87.4% for Condition 1 post-formulation. After two days, the radiochemical purity was 78.3% for Condition 2, 70.7% for Condition 3 and 27% for Condition 1 at room temperature, respectively. After six days, the radiochemical purity was 58.0% for Condition 2, 55.6% for Condition 3 and 4.3% for Condition 1 at room temperature, respectively. These results demonstrate that the preparation of ¹⁷⁷Lu-PSMA-I & T under such conditions requires heating above 90° C. for greater than 20 minutes, the incorporation conditions yield a product with radiochemical purity <97% at the time of formulation and a rapid drop in radiochemical purity over time is observed and is related to the formation of a greater number of ¹⁷⁷Lu-PSMA-I & T related impurities.

### 2. Incorporation Reaction and Formulation Compositions

The following procedures were utilized for Conditions 4-8 and Experiments 1, 3a-3d, 4a-4d, 5a-5b and 6a,6b with radiolabeling was performed using no-carrier added ¹⁷⁷LuCl₃ following the radiolabeling conditions set forth in Table 8 above. Thin layer chromatography or HPLC shows the amount of Lu-177 incorporated into the EuK-Sub-kf-iodo-y-DOTAGA was >99.5% for all reactions at completion of production at reaction temperatures of 90° C. for 30 minutes, and surprising for as short 10 min at only 60° C. HPLC showed that radiochemical purity was 99.2% post incorporation reaction for Condition 4 and the radiochemical purity for Condition 2 was 99.4% post incorporation reaction. After two days the radiochemical purity for Condition 4 is 99.1% at room temperature and after two days the radiochemical purity for Condition 5 is 99.2% at room temperature. After six days the radiochemical purity for Condition 4 is 97.6% at room temperature and Condition 5 is 96.9% at room temperature. The preparation of ¹⁷⁷Lu-PSMA-I & T following the conditions described in Condition 4 and Condition 5, demonstrates for the first time a product that is >99% pure post incorporation reaction and maintains >95% radiochemical purity over 6 days at room temperature in the Formulation Composition. In addition, ¹⁷⁷Lu-PSMA-I & T was formed under milder conditions (60° C.) for as little as 10 minutes.

3. Negative Impact of Gentisic Acid on Product Purity and Stability when Included in the Reaction Composition (Incorporation Reaction):

In FIG. 1A (Experiment 3a), the HPLC chromatogram shown for an incorporation reaction with acetate and gentisic acid has a peak at 10.2 min that is consistent with a gentisate adduct impurity. The same peak is found in the HPLC chromatograms for Condition 4, Experiments 1, 2, 3a, 3b, 3c, 3d, 5a, 6a and Condition 2. In FIGS. 1B-IC (Experiment 3c and 4d), the HPLC chromatograms show an incorporation reaction with gentisic acid and ascorbate where the gentisate adduct impurity is not consistently formed, i.e. the peak at 10.2 min, even through gentisate is present. This inconsistency in the formation the gentisate adduct impurity is consistent with the results of Experiments 3d and 4c. In FIG. 1D (Experiment 4a), the HPLC chromatogram shown for an incorporation reaction with just ascorbate the gentisate adduct impurity is not formed, i.e. the peak at 10.2 min is absent. The same result, i.e. lack of the gentisate adduct impurity, was also found for Conditions 5, 7 and 8, Experiments 4a, 4b, 5b, 6b, 7, 8, 9, 10, 11 and 12 where only ascorbate was present in the incorporation reaction. In FIGS. 1E and 1F (Condition 3 and Condition 1 respectively), the HPLC chromatogram shown for an incorporation reaction with acetate or 2-(N-morpholino) ethanesulfonic acid that does not have a peak at 10.2 min.

Therefore, to minimize impurities, gentisic acid may be excluded from the incorporation reaction.

To maintain high radiochemical purity during the incorporation reaction and during storage, ascorbate must be included in the incorporation reaction.

### 4. Positive Impact of Gentisic Acid in the Formulation Composition

It was found that inclusion of gentisic acid in combination with an ascorbate compound in the Formulation Composition at the time of or following termination of the reaction incorporating Lu-177 with EuK-Sub-kf-iodo-y-DOTAGA favorably impacted radiochemical purity of the racemic ¹⁷⁷Lu-PSMA-I & T or ¹⁷⁷Lu-PSMA I & T having an optical excess of structure 2A. That is, inclusion of gentisic acid in the Formulation Composition showed a reduced drop in radiochemical purity over time with out the formation of the gentisate adduct impurity (see FIG. 1D, which is consistent with Conditions 5, 7 and 8, Experiments 4a, 4b, 5b, 6a, 6b, 7, 8 and 12). Thus, over the 5 or 6 day evaluation period, in Experiment 3c where gentisic acid was not present in the Formulation Composition, radiochemical purity dropped 10.2%, whereas in Experiment 3d where gentisic acid was present in the Formulation Composition, radiochemical purity dropped 7.6%.

### Example 3

[¹⁷⁷Lu]-PSMA-I & T samples were prepared through an incorporation reaction as generally disclosed above. Racemic ¹⁷⁷Lu-PSMA-I & T or ¹⁷⁷Lu-PSMA I & T having an optical excess of structure 2A was produced by first preparing a Reaction Composition that contained in admixture: 1) no-carrier added ¹⁷⁷LuCl₃ in 0.04N HCl; 2) racemic EuK-Sub-kf-iodo-y-DOTAGA or EuK-Sub-kf-iodo-y-DOTAGA of structure 2A; and 3) aqueous Reaction Buffer containing sodium ascorbate concentration as a sole stabilizer compound (no gentisic acid present in the reaction buffer or incorporation reaction). The radioactive concentration of the Reaction Composition was approximately 16 GBq/mL. The concentration of sodium ascorbate in the Reaction Composition was approximately 65 mg/mL. The following Table 9 shows amount of components of the Reaction Composition:

TABLE 9

| | |
|---|---|
| Reaction buffer containing peptide precursor | 79 μL |
| ¹⁷⁷LuCl₃ in 0.04N HCl | 54 μL |
| Total reaction volume | 133 μL |

The thus prepared Reaction Composition was heated at 60° C. with mixing (shaker) for 10 minutes after which the heat source was removed. Nine aqueous Formulation Buffers (#1-9) were prepared with varying concentration of sodium ascorbate, gentisic acid and diethylenetriaminepentaacetic acid (DTPA) as set forth in the following Table 10. The pH of the Formulation Buffers #1-9 was between 6.0 to 7.0.

TABLE 10

| Formulation | Sodium ascorbate | Gentisic acid | DTPA |
|---|---|---|---|
| Buffer # | (mg/mL) | (mg/mL) | (mg/mL) |
| 1 | 66 | 28 | 0.111 |
| 2 | 66 | 21 | 0.111 |
| 3 | 66 | 14 | 0.111 |
| 4 | 49.5 | 28 | 0.111 |
| 5 | 49.5 | 21 | 0.111 |
| 6 | 49.5 | 14 | 0.111 |
| 7 | 33 | 28 | 0.111 |
| 8 | 33 | 21 | 0.111 |
| 9 | 33 | 14 | 0.111 |

Following the incorporation reaction, separate aliquots of crude Reaction Composition were each diluted (1:10) in one of Formulation Buffers #1-9. The radioactive concentration of the nine samples (i.e. Reaction Composition aliquot admixed with a Formulation Buffer) was approximately 1.45 GBq/mL. The mixture of an aliquot of crude Reaction Composition following the incorporation reaction and one of the nine Formulation Buffers is referred to below as Product Sample #1-9 (Product Samples #1-9 contain a corresponding Formula Buffer #1-9). The concentration of sodium ascorbate, gentisic acid and DTPA in the final Product Samples #1-9 is set forth in the following Table 11.

TABLE 11

| Product | Sodium ascorbate | Gentisic acid | DTPA |
|---|---|---|---|
| Sample # | (mg/mL) | (mg/mL) | (mg/mL) |
| 1 | 66 | 25 | 0.1 |
| 2 | 66 | 19 | 0.1 |
| 3 | 66 | 13 | 0.1 |
| 4 | 51 | 25 | 0.1 |
| 5 | 51 | 19 | 0.1 |
| 6 | 51 | 13 | 0.1 |
| 7 | 36 | 25 | 0.1 |
| 8 | 36 | 19 | 0.1 |
| 9 | 36 | 13 | 0.1 |

Stability testing was conducted as follows. Product Samples #1-9 were stored at room temperature and the radiochemical purity (RCP) was measured by radio-HPLC on day 0, day 2, and day 6 following preparation of each of Product Samples #1-9. The assessed radiochemical purities of each Product Samples #1-9 at each time point is reported in Table 12 below. At all concentrations of sodium ascorbate, formulations with higher amounts of gentisic acid resulted in improved stability. The largest change in radiochemical purity over the course of six days was observed in the three samples containing the lowest amount of gentisic acid (Product Sample #3, 6, 9).

TABLE 12

| | | | | | | RCP |
|---|---|---|---|---|---|---|
| Product | Sodium | Gentisic | | | | change |
| Sample | ascorbate | acid | RCP | RCP | RCP | (Day 6 − |
| # | (mg/mL) | (mg/mL) | (Day 0) | (Day 2) | (Day 6) | Day 0) |
| 1 | 66 | 25 | 99.58 | 98.23 | 96.69 | −2.89 |
| 2 | 66 | 19 | 99.59 | 98.76 | 96.03 | −3.56 |
| 3 | 66 | 13 | 99.60 | 98.57 | 94.45 | −5.15 |
| 4 | 51 | 25 | 99.36 | 99.07 | 96.23 | −3.13 |
| 5 | 51 | 19 | 99.21 | 98.72 | 95.65 | −3.56 |
| 6 | 51 | 13 | 99.11 | 98.41 | 95.40 | −3.71 |
| 7 | 36 | 25 | 99.00 | 98.82 | 96.32 | −2.68 |
| 8 | 36 | 19 | 99.34 | 98.63 | 95.91 | −3.43 |
| 9 | 36 | 13 | 99.06 | 98.29 | 94.85 | −4.21 |

It was also found that the occurrence and amount of an impurity was suppressed by including in a Product Sample a gentisate compound that had been added following the incorporation reaction. In particular, review of the radio-HPLC chromatograms of the Product Samples indicated that the improved radiochemical purity of the Product Samples with varying amounts of gentisic acid was at least partially due to suppression of an impurity which may be compound with a de-iodo form of EuK-Sub-kf-iodo-y-DOTAGA, i.e. an impurity that results from loss of iodine from the 3-iodotyrosine moiety of EuK-Sub-kf-iodo-y-DOTAGA. This impurity was observed to be formed in greater amounts in the Product Samples containing the lowest amount of gentisic acid (Product Samples #3, 6, 9). Results are summarized in the following Table 13. The % impurity as reported in Table 13 below for Days 0, 2 and 7 is the area of the impurity as measured in the radio-HPLC radiometric detector chromatogram.

TABLE 13

| | | | | | | RCP |
|---|---|---|---|---|---|---|
| Product | Sodium | Gentisic | | | | change |
| Sample | ascorbate | acid | % impurity | % impurity | % impurity | (Day 6 − |
| # | (mg/mL) | (mg/mL) | (Day 0) | (Day 2) | (Day 6) | Day 0) |
| 1 | 66 | 25 | 0.18 | ND | 1.65 | −1.47 |
| 2 | 66 | 19 | 0.20 | 0.90 | 1.92 | −1.72 |
| 3 | 66 | 13 | 0.24 | 1.21 | 2.64 | −2.40 |
| 4 | 51 | 25 | 0.39 | 0.70 | 1.34 | −0.95 |
| 5 | 51 | 19 | 0.50 | 1.05 | 2.01 | −1.51 |
| 6 | 51 | 13 | 0.44 | 1.32 | 2.60 | −2.16 |
| 7 | 36 | 25 | 0.49 | 0.90 | 1.54 | −1.05 |
| 8 | 36 | 19 | 0.51 | 1.10 | 1.84 | −1.33 |
| 9 | 36 | 13 | 0.52 | 1.42 | 2.70 | −2.18 |

In the above analyses, a Waters Acquity Arc UHPLC with a radioactivity detector and an Acquity QDa mass detector were used. A Waters XBridge BEH Phenyl-Hexyl column (150×4.6 mm, 130 Å, 3.5 μm) was used for chromatographic separation of chemical stress and radiolytic stress samples.

### Example 4

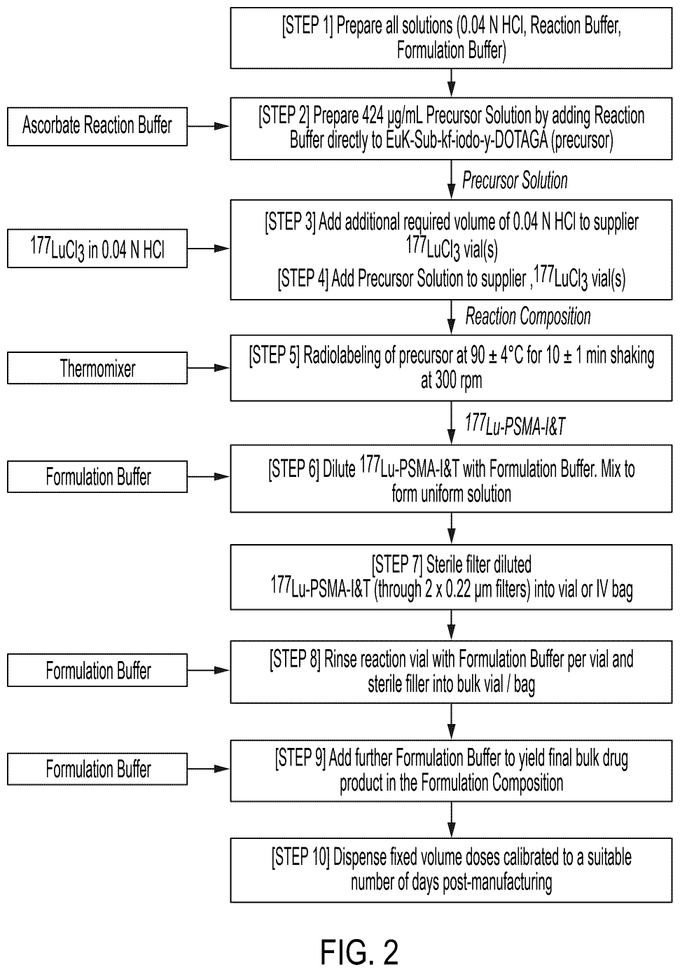

The following is a preferred preparation process as also generally depicted schematically in FIG. 2.
(1) Prepare all solutions
   a. Reaction Buffer prepared by combining sodium L-ascorbate and ascorbic acid to yield approximately 162 mg/mL Ascorbate (pH 5.5-6).
   b. Formulation Buffer prepared by combining sodium L-ascorbate and gentisic acid to yield approximately 66 mg/mL Sodium Ascorbate with approximately 28 mg/mL Gentisic acid (pH 6-7).
   c. 0.04N HCl
(2) Using Reaction Buffer, dissolve 250 μg of precursor (i.e. racemic EuK-Sub-kf-iodo-y-DOTAGA or R-isomer of EuK-Sub-kf-iodo-y-DOTAGA (compound of formula 1A)) to approximately 424 μg/mL (Precursor Solution).
(3) Add additional 0.04N HCl to the lutetium-177 vial (Reaction vial) in order to achieve a radioactive concentration of 7.8 GBq/mL).
(4) Add sufficient quantity of the Precursor Solution to the lutetium-177 vial (Reaction vial) containing no-carrier added lutetium-177 in 0.4N HCl
(5) Heat vial containing the Reaction Composition in an aluminum block with heating at 90+/−4° C. and shaking at 300 rpm for 10+/−1 min.
(6) Add Formulation Buffer into vial (Formulation vial) and mix the Reaction vial to ensure homogeneity of the contents.
(7) Sterile filter diluted ¹⁷⁷Lu-PSMA-I & T (through 2×0.22 μm filters) into a vial or IV bag.
(8) Rinse Reaction Vial with a sufficient quantity of and sterile filter rinse (through 2×0.22 μm filters) into a vial or IV bag with the diluted ¹⁷⁷Lu-PSMA-I & T.
(9) Add further Formulation Buffer to yield final bulk drug product in the Formulation Composition.
(10) Dispense fixed volume doses calibrated to a suitable number of days post-manufacturing.

The above process also can be utilized to prepare ¹⁷⁷Lu-PSMA I & T that is substantially optically enriched with or is an enantiomerically pure mixture of the S-isomer of ¹⁷⁷Lu-PSMA I & T (compound of structure 2B above) by use of the S-isomer of EuK-Sub-kf-iodo-y-DOTAGA (compound of structure 1B)).

### Example 5: Treatment Protocol

A human male patient is selected for treatment after being diagnosed with metastatic castration-resistant prostate cancer (such as manifested by progression of the disease despite surgical or chemical castration) who have progressed following treatment first line androgen receptor-axis-targeted (ARAT) therapies.

¹⁷⁷Lu-PSMA I & T having an optical excess of structure 2A in a sterile aqueous solution is administered by intravenous injection. The dosing regimen may include four infusions of 6.8 GBq+/−10% each, administered 8 weeks apart.

### INCORPORATION BY REFERENCE

The entire contents of all patents, published patent applications and other disclosures cited herein are hereby expressly incorporated herein in their entireties by reference.

While this invention has been particularly shown and described with references to preferred embodiments thereof, it will be understood by those skilled in the art that various changes in form and details may be made therein without departing from the scope of the invention encompassed by the appended claims.

1-53. (canceled)

54. A method of treating prostate cancer in a subject, comprising:
a) assessing a pharmaceutical composition comprising ¹⁷⁷Lu-PSMA I & T for presence of a deiodination impurity; and
b) administering the pharmaceutical composition to the subject if the amount of the deiodination impurity in the pharmaceutical composition is assessed to be less than 6%.

55. The method of claim 54, wherein the pharmaceutical composition is assessed one day or more following preparation of the pharmaceutical composition.

56. The method of claim 54, wherein the pharmaceutical composition is assessed three days or more following preparation of the pharmaceutical composition.

57. The method of claim 54, wherein the targeted amount of the deiodination impurity is assessed to be less than 5%.

58. The method of claim 54, wherein the targeted amount of the deiodination impurity is assessed to be less than 3%.

59. The method of claim 54, wherein the targeted amount of the deiodination impurity is assessed to be less than 2%.

60. The method of claim 54, wherein the targeted amount of the deiodination impurity is assessed to be less than 1%.

61. The method of claim 54, wherein the amount of the deiodination impurity is determined by reverse-phase HPLC.

62. The method of claim 61, wherein the reverse-phase HPLC is reverse-phase HPLC with UV and radiometric detection.

63. The method of claim 54, wherein the pharmaceutical composition is maintained at 30° C.
